SEQUENCE LISTING (14 sequences)

SEQ ID NO: 1 - DNA, 20 bp, synthetic construct
  source 1..20: mol_type = other DNA
cgcccttaacagcagtcttc

SEQ ID NO: 2 - DNA, 20 bp, synthetic construct
  source 1..20: mol_type = other DNA
gccctccgtatgcttaaacc

SEQ ID NO: 3 - RNA, 20 nt, synthetic construct
  source 1..20: mol_type = other RNA
agagtttgatcctggctcag

SEQ ID NO: 4 - RNA, 23 nt, synthetic construct
  source 1..23: mol_type = other RNA
cttgtgcgggcccccgtcaattc

SEQ ID NO: 5 - DNA, 24 bp, synthetic construct
  source 1..24: mol_type = other DNA
ttgttaccaactgggacgacatgg

SEQ ID NO: 6 - DNA, 24 bp, synthetic construct
  source 1..24: mol_type = other DNA
ctggggtgttgaaggtctcaaaca

SEQ ID NO: 7 - DNA, 20 bp, synthetic construct
  source 1..20: mol_type = other DNA
gggagggtcaaatgaagaca

SEQ ID NO: 8 - DNA, 20 bp, synthetic construct
  source 1..20: mol_type = other DNA
ggcattcctgctggttacat

SEQ ID NO: 9 - DNA, 20 bp, synthetic construct
  source 1..20: mol_type = other DNA
attccatcagtttcctatct

SEQ ID NO: 10 - DNA, 18 bp, synthetic construct
  source 1..18: mol_type = other DNA
accaggacctttcttgac

SEQ ID NO: 11 - DNA, 18 bp, synthetic construct
  source 1..18: mol_type = other DNA
atggctaccgtggtgtca

SEQ ID NO: 12 - DNA, 20 bp, synthetic construct
  source 1..20: mol_type = other DNA
cggtctccacggttgtagtt

SEQ ID NO: 13 - DNA, 20 bp, synthetic construct
  source 1..20: mol_type = other DNA
taatgctgttggtggtcctg

SEQ ID NO: 14 - DNA, 20 bp, synthetic construct
  source 1..20: mol_type = other DNA
cagccacggttgttacactg

(19) United States
(12) Patent Application Publication
(10) Pub. No.: US 2024/0350456 A1
(43) Pub. Date: Oct. 24, 2024

(54) COMPOSITIONS AND METHODS FOR DIAGNOSING AND TREATING INFLAMMATORY CONDITIONS

(71) Applicant: RUTGERS, THE STATE UNIVERSITY OF NEW JERSEY, New Brunswick, NJ (US)

(72) Inventors: Ronaldo P. Ferraris, Millington, NJ (US); Nan Gao, Basking Ridge, NJ (US); Wei Vivian Li, Riverside, CA (US)

(73) Assignee: RUTGERS, THE STATE UNIVERSITY OF NEW JERSEY, New Brunswick, NJ (US)

(21) Appl. No.: 18/631,821

(22) Filed: Apr. 10, 2024

Related U.S. Application Data
(60) Provisional application No. 63/495,426, filed on Apr. 11, 2023.

(51) Int. Cl. A61K 31/4045; A61K 31/047; A61K 31/132; A61K 31/145; A61K 31/19; A61K 31/194; A61K 31/195; A61K 31/197; A61K 31/198; A61K 31/20; A61K 31/205; A61K 31/216; A61K 31/221; A61K 31/223; A61K 31/40; A61K 31/401; A61K 31/4015; A61K 31/404; A61K 31/405; A61K 31/4172; A61K 31/4188; A61K 31/455; A61K 31/51; A61K 31/513; A61K 31/522; A61K 31/609; A61K 31/66; A61K 31/685; A61K 31/7012; A61K 31/706; A61K 31/7068; A61K 31/7072; A61K 31/7076; A61K 31/708; A61P 1/12; C12Q 1/6883; G01N 33/68

(52) U.S. Cl. CPC A61K 31/4045; A61K 31/047; A61K 31/132; A61K 31/145; A61K 31/19; A61K 31/194; A61K 31/195; A61K 31/197; A61K 31/198; A61K 31/20; A61K 31/205; A61K 31/216; A61K 31/221; A61K 31/223; A61K 31/40; A61K 31/401; A61K 31/4015; A61K 31/404; A61K 31/405; A61K 31/4172; A61K 31/4188; A61K 31/455; A61K 31/51; A61K 31/513; A61K 31/522; A61K 31/609; A61K 31/66; A61K 31/685; A61K 31/7012; A61K 31/706; A61K 31/7068; A61K 31/7072; A61K 31/7076; A61K 31/708; A61P 1/12; C12Q 1/6883; G01N 33/6893; C12Q 2600/158; G01N 2800/06

(57) ABSTRACT

Compositions and methods useful for the diagnosis and treatment of leaky gut syndrome and IBD including Crohn's disease are disclosed.

### CROSS-REFERENCE TO RELATED APPLICATIONS

This application claims priority of U.S. Provisional Application No. 63/495,426 filed Apr. 11, 2023, the entire contents being incorporated herein by reference as though set forth in full.

### FEDERALLY SPONSORED RESEARCH AND DEVELOPMENT

This invention was made with government support under grant numbers AT010243 and DK119198 awarded by The National Institutes of Health and grant number ISO 1754783 awarded by the National Science Foundation. The government has certain rights in the invention.

### INCORPORATION-BY-REFERENCE OF MATERIAL SUBMITTED IN ELECTRONIC FORM

Incorporated herein by reference in its entirety is the sequence listing submitted via EFS-Web as a text file named RUT-112-US_SeqList.xml, created, Apr. 10, 2024 and having a size of 13,375 bytes.

### FIELD OF INVENTION

The present invention relates the fields of the diagnosis and treatment of inflammatory diseases. More specifically, the invention provides biomarker diagnostics and nutraceutical therapeutics comprising LGG-regulated metabolites.

### BACKGROUND OF THE INVENTION

Lactobacillus (renamed Lacticaseibacillus) rhamnosus GG (LGG, ATCC 53103) is a rod-shaped Gram-positive bacterium isolated in 1983 from human feces which exhibits high resistance to bile and gastric acids, excellent luminal growth, and firm adhesion to intestinal epithelial cells (IEC). LGG diminishes symptoms of antibiotic-associated and rotavirus-related diarrhea, with evidence related to symptom reduction in necrotizing enterocolitis, irritable bowel syndrome (IBS), and autoimmune diseases. The overall mechanisms underlying LGG's beneficial effects are mostly unknown.

Impaired gut barrier or leaky gut syndrome is an increasingly recognized condition associated with a large array of intestinal and systemic diseases, including inflammatory bowel diseases (IBD), systemic lupus erythematosus, arthritis, and type 1 diabetes (Kinashi and Hase, 2021; Mu et al., 2017; Silverman et al., 2019). IBD has long been linked with impaired intestinal permeability, with IBD patients displaying an increased paracellular permeability with tight junction (TJ) abnormalities (Yoo and Donowitz, 2019).

We recently reported that LGG profoundly changes the host intestinal luminal metabolome while excluding gut colonization of pathobiont bacteria (Kim et al., 2021). The sources of these related metabolites are products from diet-host-microbiota interactions. Although LGG is known to synthesize tryptophan (trp) (Ceapa et al., 2016), and may utilize the Aryl hydrocarbon receptor (AhR) signaling to exert its effects on host (Zelante et al., 2013), the identity and health impact of LGG-dependent metabolites remain poorly characterized in vivo.

Earlier investigations overall failed to establish a mechanistic link between LGG-driven metabolites and host gut physiology. Clearly, a need exists to diagnose and treat leaky gut.

### SUMMARY OF THE INVENTION

In one aspect, the invention includes methods for diagnosing leaky gut in a subject comprising: (a) measuring in a biological fluid sample of the subject the expression level of a gene, gene fragment, gene transcript or expression product selected from the biomarkers identified in FIG. 8K, 9H, 14, and (b) comparing levels of said subject's selected gene, gene fragment, gene transcript or expression product with the level of the same gene, gene fragment, gene transcript or expression product in the biological fluid of a reference or control subject, wherein changes in expression of the subject's selected gene, gene fragment, gene transcript or expression products from those of the reference or control correlates with a diagnosis of leaky gut.

In another aspect, the invention includes methods for diagnosing leaky gut in a subject comprising: (a) measuring in a biological fluid sample of the subject the expression level of a protein, peptide fragment or expression product thereof selected from at least one of the biomarkers identified in FIG. 8K. 9H, 14,; and (b) comparing said subject's expression level of the selected biomarker with the level of the same biomarker in the biological fluid of a reference or control subject, wherein changes in expression of the subject's selected biomarker from those of the reference or control correlates with a diagnosis of leaky gut.

In certain embodiments, the biomarkers are selected from i. L-argininosuccinic acid (ASA), ii. argininosuccinate synthase 1 (ASS1), iii. Argininosuccinate lyase (ASL), and Nitric oxide synthase (NOS2). In certain embodiments, the biomarkers include all of (i)-(iv). In certain embodiments, said change in expression level of each said selected protein or peptide fragment comprises an upregulation in comparison to said reference or control or a downregulation in comparison to said reference or control. In certain embodiments, the method further comprises measuring the expression level of an expression product, protein, or peptide fragment selected from FIG. 8K, 9H, or 14, or a polynucleotide or oligonucleotide sequence, which hybridizes to a gene, gene fragment, gene transcript or expression product selected from FIG. 8K, 9H, 14.

In certain embodiments, the methods further comprise administering to the patient a therapeutic agent that reduces inflammation. In certain embodiments, the agent is at least one of nutraceutical, anti-inflammatory drug, antibiotic, immunomodulator, anti-diarrheal medication, pain reliever, iron supplement, and calcium and vitamin D supplement. In certain embodiments, the nutraceutical is selected from one or more of the compounds listed in Tables 2 or 3. In certain embodiments, the nutraceutical is one or more of indole acetamide (IAM), methynicotinamide (MNA), carnosine (CARN), and indole-propionic acid (IPA). In certain embodiments, the nutraceutical is all of IAM, MNA, CARN, and IPA. In certain embodiments, the method further comprises administering at least one additional therapeutic agent.

In certain embodiments, the agent is administered orally, intraperitoneally, intravenously, or intramuscular. In certain embodiments, steps a) and b) are repeated after administering the therapeutic agent.

In another aspect, the invention comprises methods for treating an inflammatory disease in a subject in need thereof, the method comprising administering one or more of the nutraceutical compounds listed in Tables 2 or 3. In yet another aspect, the invention comprises methods for treating leaky gut syndrome in a subject in need thereof, the method comprising administering one or more of the nutraceutical compounds listed in Tables 2 or 3. In certain embodiments, the nutraceutical is one or more of indole acetamide (IAM), methynicotinamide (MNA), carnosine (CARN), and indole-propionic acid (IPA). In certain embodiments, the nutraceutical is all of IAM, MNA, CARN, and IPA. In certain embodiments, the method further comprises administering at least one additional therapeutic agent. In certain embodiments, the agent is administered orally, intraperitoneally, intravenously, or intramuscular. In certain embodiments, steps a) and b) are repeated after administering the therapeutic agent.

In certain embodiments the patient has inflammatory bowel disease and/or Type I diabetes.

In another embodiment, the invention comprises kits or reagents for performing the methods described herein.

Still other aspects and advantages of these compositions and methods are readily apparent and described further in the following detailed description of the invention.

### BRIEF DESCRIPTION OF DRAWINGS

FIG. 1A-1D. LGG-inoculated mice were successfully and rapidly colonized with relatively equivalent LGG abundance throughout the 3-week colonization duration. FIG. 1A, % change of body weight over 6-week experimental period. FIG. 1B, feeding rate (g/cage) of phosphate buffered saline (PBS) trp− (blue symbols), PBS trp+ (purple), LGG trp− (green), LGG trp+ (orange) mice. FIG. 1C, Cq number of RT-PCR analysis using Universal 16S primers of fecal samples collected during pre-inoculation (PRE), and at 1, 2 and 3 weeks post-inoculation. FIG. 1D, Cq number of RT-PCR using LGG specific primers at the same time points.

FIG. 2A-2J. Effect of LGG and dietary tryptophan on the ileal transcriptome. FIG. 2A depicts a 2×2 experimental design of germ-free (GF, gavaged with PBS) and LGG-monoinoculated mice (LGG) fed a trp-sufficient (trp+) or trp-deficient (trp-) diet for 3 wk after inoculation. Arrows=fecal collection, gavage and sacrifice (Sac). FIG. 2B, Principal component analysis (PCA) of ileal transcriptome from mice fed a trp-diet and gavaged with PBS (PBS−, blue), fed trp+ and with PBS (PBS+, purple), fed trp− and with LGG (LGG−, green) and fed trp+ and with LGG (LGG+, orange). FIG. 2C. Heatmap of results from bulk sequencing of ileal RNA from PBS−, PBS+, LGG− and LGG+ (n=5 each group). Only significantly different genes are shown (False discovery rate (FDR)<0.05). FIG. 2D, Venn diagram of numbers of ileal transcripts (FC>1.5, FDR<0.05) that represent trp effect without (PBS+ vs PBS−) and with LGG (LGG+ vs LGG−). FIG. 2E, Ileal transcripts regulated by LGG without (LGG− vs PBS−) and with trp (LGG+ vs PBS+). FIG. 2F, Volcano plot of numbers of genes up- and down-regulated (FC>1.5, FDR<0.05) by LGG without trp. FIG. 2G, genes up- and down-regulated by LGG with trp. FIG. 2H. Normalized enrichment score (NES) following GSEA when comparing transcriptomes from trp-sufficient mice colonized with LGG (LGG+) and without (PBS+). FIG. 2I, Leading edge graphs depicting nonrandom distribution of genes in selected pathways TJ, brush-border and fatty acid β-oxidation from LGG+vs PBS+. Many of the enriched genes increase with LGG. FIG. 2J, immunostaining against Trefoil factor 3 (TFF3, green), alkaline phosphatase (AP, red), lysozyme 1 (LYZ1, white), Dapi (blue). Scale bar, 50 μm. Brush-border marker AP expression decreased in trp-sufficient mice, but LGG increased its expression. The goblet cell marker TFF3 decreased with LGG while the Paneth cell marker LYZ1 is reduced in crypts of LGG+ mice.

FIG. 3A-3B. Impact of LGG on tryptophan regulation of ileal transcriptome. FIG. 2A. Volcano plot of numbers of genes up- and down-regulated (FC>1.5, FDR<0.05) by trp without LGG, and of (FIG. 2B) genes up- and down-regulated (FC>1.5, FDR<0.05) by trp with LGG.

FIG. 4A-4L. LGG and tryptophan enhance enterocyte but inhibit Paneth cell transcriptome. FIG. 4A, Targeted, representative heatmaps of ilcal TJ, brush-border and fatty acid β-oxidation transcripts significantly different (FDR<0.05) among PBS− (blue), PBS+ (purple), LGG− (green) and LGG+ (orange) mice (n=5). FIG. 4B, mRNA expression (Reads Per Kilobase of transcript, per Million mapped reads, or RPKM) of representative brush-border genes sucrase-isomaltase (Si) and sodium-dependent glucose transporter (Slc5a1). FIG. 4C, RPKM of TJ genes zonula occludens (ZO1 or Tjp1) and occludin (Ocln). FIG. 4D, Representative heatmaps of ileal TJ, brush-border and fatty acid β-oxidation transcripts in GF (blue), LGG-monocolonized (orange), R. gnavus-monocolonized (red) and SPF (yellow) mice (n=4-5 each group) indicating LGG effects are specific. FIG. 4E. Partial heatmaps of transcripts expressed in goblet (top) and Paneth (bottom) cells as defined by GSEA. RPKM of representative transcripts of goblet (FIG. 4F), Paneth (FIG. 4G) and stem (FIG. 4H) cells. FIG. 4I, experimental design, conventional mouse intestine perfused with saline (Ringer) or LGG for 4 h, followed by RNA sequencing. FIG. 4J, PCA plot indicating separation of saline-(blue) and LGG-perfused (orange) mice (n=4). FIG. 4K. Volcano plot of ileal transcriptome perfused with or without LGG. Significantly-altered expression shown in red (|FC|>1.5, FDR<0.05). Like monocolonization, perfusion of LGG reduces transcripts of the defensin family. Interestingly, Cyp1a1, a biomarker of AhR activation increases with LGG. FIG. 4L, RPKM of representative transcripts.

FIG. 5. Tryptophan dependence of LGG's potential effect on Aryl hydrocarbon receptor (AhR) signaling. Expression (RPKM) of the AhR nuclear translocator Arnt increases modestly with LGG (gray bars) in the absence of trp, but markedly with trp. * P<0.05. *** P<0.001.

FIG. 6A-6J. LGG metabolome is differentially distributed mainly into the luminal and serum but not liver compartments. FIG. 6A-6B, heatmap of fecal metabolites analyzed by LCMS in negative (top) and positive (bottom) modes. There were significant changes in 121 (88 negative and 62 positive, with 29 metabolites appearing in both modes) fecal metabolites. There were 106 (negative) and 69 (positive) serum metabolites as well as 104 (negative) and 75 (positive) liver metabolites (not shown). Darker brown color indicates greater up-regulation; blue, down-regulation. FIG. 6C, PCA of fecal metabolites (negative mode) over time, of samples obtained one-week before (left), then one-week (middle) and three-weeks (right) after LGG inoculation (n=5). PCA of serum (FIG. 6D) and liver (FIG. 6E) metabolites three weeks after LGG inoculation. FIG. 6F, Venn diagram depicting number of fecal (red), serum (green) and liver (blue) metabolites (negative mode) significantly different between LGG+ vs PBS+ mice (|FC|>1.5, FDR<0.05). Thus, 27 fecal metabolites differed between LGG+ and PBS+ mice, of which two are also in the serum. Variables Important in the Projection (VIP) scores of fecal metabolites driving the inter-group differences between LGG and GF mice deficient in trp (FIG. 6G, LGG− vs. PBS−) as well as between LGG and GF mice fed trp-sufficient diets (FIG. 6H, LGG+ vs. PBS+). VIP scores of serum metabolites between LGG− and PBS− (FIG. 6I) as well as between LGG+ vs. PBS+ (FIG. 6J).

FIG. 7A-7C. Effect of LGG is greatest on the fecal metabolome (positive mode). FIG. 7A. PCA of fecal metabolites (positive mode) over time, of samples obtained one-week before (left), then one-week (middle) and three-weeks (right) after LGG inoculation (n=5). PCA of serum (FIG. 7B) and liver (FIG. 7C) metabolites three weeks after LGG inoculation.

FIG. 8A-8K. Tryptophan-dependent modification by LGG of host luminal and serum metabolites. FIG. 8A, Dietary trp can be metabolized by host via the kynurenine (KYN) pathway or converted by gut bacteria into indoles and indole derivatives, some of which can transported into the serum. FIG. 8B, Fecal (F) metabolites that increase with LGG, trp, or by a synergism between LGG and trp (PBS, left; LGG, right bars). FIG. 8C, trp-derived serum(S) metabolites that increase over time and primarily with trp, except for IPA and indoleacrylic acid whose levels increase further with LGG in a synergistic manner. FIG. 8D. In contrast, in the liver (L), trp metabolites increase only with dietary trp, independent of LGG. Fecal (FIG. 8E) and serum (FIG. 8F) metabolites which are not derivatives of trp whose levels increased only in trp-sufficient. LGG mice indicating LGG-trp synergy. FIG. 8G, serum metabolites whose levels increase only in trp-deficient mice with LGG. FIG. 8H, fecal metabolites whose concentrations decrease with LGG independent of dietary trp. FIG. 8I, serum metabolites that increase with dietary trp but then decrease with LGG. FIG. 8J, serum metabolite that tends to decrease with LGG independent of trp. FIG. 8K. serum metabolites suppressed by LGG.

FIG. 9A-9H. Changes in levels of specific liver, serum and fecal metabolites are tightly correlated with changes in expression of ileal transcripts. FIG. 9A. Heatmap of numbers (dark blue=0, dark red=4000) of ileal transcripts whose levels are significantly correlated with levels of specific metabolites (negative (−) and positive (+) modes) in the liver (L), serum(S) and feces (F) of LGG trp-compared with PBS trp-mice. These are metabolite-transcript correlations regulated by LGG without trp. FIG. 9B, metabolite-transcript correlations regulated by LGG with trp (LGG trp+ compared with PBS trp+ mice). FIG. 9C, metabolite-transcript correlations regulated by dietary trp in mice with LGG (LGG trp+ compared with LGG trp-). Note the large increase in number of transcripts regulated when both LGG and trp are present. FIG. 9D, Correlations between 10 representative transcripts with individual metabolites. Transcripts modulated by fecal IAM and 5-hydroxy-L-trp whose levels increase synergistically with trp and LGG (see FIG. 8B), as well as with fecal indolelactic acid whose levels increase with LGG regardless of dietary trp. Blue line indicates positive correlation; red, negative correlation. A thick line indicates regulation by both LGG in trp sufficient mice (LGG trp+compared with PBS trp+), and by trp in LGG mice (LGG trp+compared with LGG trp-), a thin line by only one comparison. Colored circles on genes depict functional groups (light blue=TJ, dark blue=brush-border, gray=proliferation, orange=fatty acid synthesis and metabolism, green=lipid beta oxidation, yellow=goblet cell, and pink=Paneth cell marker. FIG. 9E, Correlations between representative transcripts and serum IAM, indolelactic acid and 5-methyl-trp whose levels increase primarily with dietary trp. Correlations between representative transcripts and serum indoles (FIG. 9F) and nonindole metabolites (FIG. 9G) whose levels increase synergistically with LGG and trp. FIG. 9H, Correlations between representative transcripts and serum metabolites whose levels decrease with LGG. These metabolites are strongly correlated with transcripts but oppose the regulation of mostly indole and trp-derivatives that enhance expression of many cell types and pathways.

FIG. 10. Correlations between serum as well as fecal indole-3-carboxyaldehyde with ileal transcripts. Serum indole-3-carboxyaldehyde are correlated with representative transcripts from the TJ (light blue), brush border (dark blue), AhR (gray), goblet cells (yellow), triglyceride synthesis (orange) and fatty acid oxidation (green). Fecal indole-3-carboxyaldehyde is correlated with similar transcripts, except for those from the brush border. Thus, the location of the metabolite impacts its effect on the ileal transcriptome.

FIG. 11A-11R. Regulation of gut barrier and cell differentiation by serum and fecal LGG metabolites. FIG. 11A, targeted heatmap of numbers (dark blue=0, dark red=14) of major TJ transcripts whose ileal levels are significantly correlated with levels of specific metabolites (negative (−) and positive (+) modes) in the liver (L), serum(S) and feces (F) of LGG trp-compared with PBS trp-mice. These are metabolite-transcript correlations regulated by LGG without trp. FIG. 11B, targeted heatmap of correlations between metabolites and TJ genes regulated by LGG with trp (LGG trp+compared with PBS trp+ mice). Metabolites regulating a greater number of genes are found in the fecal compared to liver and serum compartments. Ten representative fecal and serum metabolites significantly correlated with ileal mRNA expression of TJ genes Ocln (FIG. 11C) and Tjp1 (ZO1, FIG. 11D) as well as brush-border transporter Slc5a1 (SGLT1, FIG. 11E). Blue line=positive correlation, red=negative. A thick line indicates regulation in two correlations; thin, one comparison. FIG. 11F, correlation graphs between the level of selected serum metabolites and mRNA expression of Ocln. FIG. 11G, experimental design of organoid experiments to evaluate metabolite efficacy and validate correlations. LPS=lipopolysaccharide (1 μg/mL, to disrupt the gut barrier) was added to CCM media (basolateral side) with and without metabolites: IPA (100 μM), IAM (200 μM), carnosine (CARN; 20 mM), methylnicotinamide (MNA, 88 μM) or L-argininosuccinate (ASA. 1 μM) (n=4-7 each group, except control, n=14). FIG. 11H, Representative images of immunofluorescent staining of ZO-1 (left column) and Ocln (right). mRNA expression levels of Tjp1 (ZO-1) (FIG. 11I), and Ocln (FIG. 11J) in control, LPS, LPS+ metabolite and metabolite-treated organoids. Treatment duration=48 hr. * P<0.05, ** P<0.01 compared with control. Orange bars are organoids treated with LPS plus metabolites; blue, treated with metabolite alone. Permeability of organoids treated with LPS+ metabolites positively correlated with mRNA expression of Ocln (FIG. 11K), or LPS+a metabolite negatively correlated with Ocln (FIG. 11L). FIG. 11M, Representative images of permeability assay. FIG. 11N, Representative fecal and serum metabolites correlated to the expression of the Paneth cell-specific Lyz1. FIG. 11O, correlation graphs between the serum metabolite MNA and mRNA expression of Slc5a1 (brush-border biomarker), Lyz1 (Paneth) and Tff3 (goblet). FIG. 11P. mRNA expression level of Tff3 (left), and Lyz1 (right) with selected negatively-correlated metabolites. FIG. 11Q. Representative photograph of immunofluorescent staining of TFF3 (left) and LYZ1 (right) of control organoids and those treated with IPA for 48 h. FIG. 11R, Number of TFF3-positive (left) and LYZ1-positive (right) cells per organoid area (mm2).

FIG. 12. Correlations between TJ transcripts MarvelD2 (top) and Cgn (bottom) with serum as well as fecal metabolites. Blue line indicates positive correlation; red, negative correlation. A thick line indicates regulation by both LGG in trp sufficient mice (LGG trp+compared with PBS trp+), and by trp in LGG mice (LGG trp+compared with LGG trp-), a thin line by only one comparison. TJ genes are correlated with numerous fecal metabolites that can differ from those in the serum. Moreover, different TJ transcripts may be correlated with the same or dissimilar metabolites.

FIG. 13A-13M. IPA requires Ocln to alleviate LPS-induced leaky epithelium. FIG. 13A, experimental design. Organoids were derived from tamoxifen-inducible, intestine-specific Ocln-knockout (KO) mice generated by crossing a floxed Ocln^{fl/fl} with Villin-CreERT2 mice. FIG. 13B, Representative images and FIG. 13C, quantitative analysis of permeability assay. LPS was added to CCM media (basolateral side) with and without IPA (100 M) and IAM (200 μM) (n=3 mice). *** P<0.001, compared with WT control, †P<0.05 compared with KO-control. FIG. 13D, Representative images of immunofluorescent staining of Ocln in WT control and Ocln KO conventional mice, L=lumen. MNA improves but ASA exacerbates dextran sodium sulfate (DSS)-induced leaky gut. FIG. 13E, experimental design, evaluation in vivo of metabolites (Met). Methylnicotinamide (MNA. 200 mg/kg body weight) and L-argininosuccinate (ASA. 3.6 mg/kg) were administered (i.p.) twice 1-day before and 3 days after DSS treatment p.o. 4 kDa fluorescein isothiocyanate (FITC)-dextran was gavaged 4 h before sacrifice. FIG. 13F, body weight of DSS-treated mice with and without metabolites, FIG. 13G, representative images of Ocln-stained colon sections (green; left panel) and ECad (red; middle). Merged images (yellow; right panel) depict Ocln location in TJ of MNA-treated but not in DSS-only and ASA-treated colon (arrows). Quantitative fluorescent intensity of Ocln (FIG. 13H) and ECad (FIG. 13I) in the TJ, relative to DSS only controls. FIG. 13J, DSS scoring. FIG. 13K, representative H&E images of DSS, DSS+ MNA and DSS+ASA colon sections. FIG. 13L, In vivo permeability as determined by fluorescent intensity of FITC in serum. FIG. 13M, Serum level of interleukin-6.

FIG. 14. Analysis of ASS1, ASL, and NOS2 levels in untreated mice (white bar) and mice treated with LGG (gray bar) fed a trp− or trp+ diet.

FIG. 15 Nitric oxide levels in polarized Caco-2 cells after 24 h exposure to 5% LGG supernatant (left panel), and after exposure to arginine, citrulline and ornithine which are typical metabolites of LGG (right panel).

FIG. 16. Effect of LGG and its metabolites on transepithelial electrical resistance (TEER) of confluent Caco-2 cells. Supernatant was collected from LGG culture then diluted with medium (1.25, 2.5 and 5%) now likely containing metabolites synthesized in vitro. Confluent Caco2 BBE monolayer was then given LGG supernatant (spent media), at different luminal concentrations.

FIG. 17: Effect of a representative LGG metabolite, methyl-nicotinamide (MNA) on TEER of a confluent Caco2 BBE monolayer. MNA was applied from Day-5 at different concentrations on the luminal (L) or basolateral (BL) compartments. The monolayer was disrupted by 3% DSS in the luminal side on days 5-8.

FIG. 18A-18B. Analysis of the effect of a beneficial LGG metabolite MNA (FIG. 18A) and a deleterious one, ASA (FIG. 18B), provided i.p. every other day since Day 1, on the body weight of a leaky gut mouse model. A moderate amount of DSS (3%) creates a leaky gut model.

FIG. 19A-19C. Levels of vitamin B3 and indole metabolites tend to be low in IBD patients which have leaky gut. FIG. 19A. Diagram illustrating convergence of the metabolic pathways of dietary tryptophan (trp, top, light blue shade) and vitamin B3 (nicotinic acid (NA, middle, purple shade) or nicotinamide (NAM, bottom, light orange)) that are broken down into various metabolites inside the cell. FIG. 19B, Analysis of levels of various indoles (which are made by bacteria from tryptophan inside human intestines) in serum of IBD (inflammatory bowel disease), CD (Chron's Disease), UC (ulcerative colitis) compared to CN (control). FIG. 19C, Levels of methylnicotinamide (MNA), xanthurenate (XAN), and kynurenine (KYN) are also all low in the serum IBD, UC and CD patients.

FIG. 20A-20B. In vitro screening of metabolites effective against leaky gut. FIG. 20A, Diagram of the in vitro transwell method used to evaluate efficacy of metabolites in protecting the leaky gut in human-derived Caco-2 cells. The luminal compartment represents the food side, and the basolateral the blood side. FIG. 20B, Transepithelial electrical resistance (TEER) measurements, and FITC-dextran measurements, evaluate the leakiness of the tight junction that regulates the pore (top) and leak (bottom) pathways of the gut. Ions (red), small molecules (blue), FITC-dextran (orange).

FIG. 21A-21D. Efficacy of tryptophan and vitamin B3 metabolites in protecting the gut barrier in vitro. FIG. 21A, TEER measurements of metabolites placed on luminal side of transwells for 72 hours. After 48 hours, the barrier was disrupted by the toxin of clostrium difficile (Cdiff). FIG. 21B, Integrated results of area under the curve using data from FIG. 21A. FIG. 21C, FITC-dextran permeability analysis of metabolites applied luminally. FIG. 21D, FITC-dextran permeability analysis of metabolites applied basolaterally.

FIG. 22A-22B. Efficacy of microbiota-derived indole metabolites in protecting the gut barrier. FIG. 22A, Integrated results of TEER measurements of metabolites placed on luminal side of transwells for 72 hours. After 48 hours, the barrier was disrupted by the toxin of clostrium difficile (Cdiff) FIG. 22B, FITC-dextran permeability analysis of metabolites from FIG. 22A applied luminally.

FIG. 23A-23D. MNA dampens the effects of DSS-induced leaky gut colitis on body weight by reducing inflammation, barrier disruption and tissue damage. FIG. 23A, Analysis of plasma ovalbumin levels after oral ovalbumin gavage in mice treated with exogenous MNA or PBS. FIG. 23B. Analysis of fecal lipocalin levels in mice treated with PBS or MNA with or without DSS. FIG. 23C. Representative histology sections of mice treated with PBS or MNA with or without DSS. FIG. 23D, quantification of mouse histology sections scored for colitis index.

FIG. 24A-24C. MNA increases mRNA and protein levels of important tight junction components to seal and protects the gut barrier. FIG. 24A, Analysis of zonula occludens (Tjp1) and occluding (Ocln) mRNA levels after treatment with PBS or MNA with or without DSS. FIG. 24B, Western blot analysis and quantification of Ocln protein levels after treatment with PBS or MNA with or without DSS. FIG. 24C, Quantified levels of occluding in the ileum (top) and colon (bottom) using immunofluorescence antibody staining.

FIG. 25A-25B. LGG can make MNA precursors while the host responds by upregulating trp and vitamin B3 metabolism pathways. FIG. 25A, KYN and NAD levels in LGG cultured alone in aerobic and anerobic conditions. FIG. 25B, heatmaps depicting the increase in genes involved in vitamin B3/nicotamide (left) and tryptophan (right) metabolic pathways.

FIG. 26. Arginine/urea cycle.

FIG. 27A-27B. Human IBD data. FIG. 27A, Data showing that ASS (which makes ASA) increases in inflamed tissues when compared to an adjoining colon tissue that is not inflamed and to healthy controls. FIG. 27B, Data showing that ASL (which degrades ASA) levels remain the same.

FIG. 28A-28B. Analysis of Mouse model of DSS colitis. FIG. 28A, Serum levels of 25 proteins, including ASA, in the mouse model of DSS. FIG. 28B, Stained histology sections of mice colons after treatment with PBS or DSS.

FIG. 29A-29B. Analysis of LGG bacteria. FIG. 29A, analysis of protein levels of LGG when cultured in bacteria growth media. FIG. 29B, Histology sections of illeum and colons of mice monoconolized with LGG compared to PBS treated mice.

FIG. 30. Analysis of Caco2 cells cultures treated with various LGG-derived metabolites. Abbreviations: Veh=vehicle, IAM=indole-3-acetamide, ICA=indole-3-carboxyaldehyde, ICC=indole-3-carboxylic acid, ILA=indole-3-lactic acid, IAA=indole-3-acetic acid, ARG=arginine, ORN=ornithine, ASA=argininosuccinic acid, ASP=aspartic acid.

FIG. 31A-31D. Human IBD metabolite data analyzing perturbed arginine metabolism compared to controls.

### DETAILED DESCRIPTION OF THE INVENTION

Lacticaseibacillus rhamnosus GG (LGG) is a widely consumed probiotic, but it remains unclear how LGG shapes host metabolome under different dietary conditions. Here, we monocolonized LGG in germ-free mice fed regular or tryptophan-deficient diets, and uncovered LGG-dependent and tryptophan-regulated metabolomes across different tissues. Untargeted transcriptomic analysis revealed a strong synergy between LGG and dietary tryptophan in promoting enterocyte maturation, in particular the production of tight junction (TJ) components. Using an innovative metabolome-transcriptome correlation analysis, we uncovered, then validated, specific serum and intestinal metabolites that promote or impair barrier function. In addition to enhancing the production of beneficial metabolites, LGG, in conjunction with dietary tryptophan, significantly reduces deleterious metabolites that reduce TJ gene expression and impair barrier function in and ex vivo. These results demonstrate for the first time that LGG synergizes with a key dietary component to regulate gut epithelial differentiation and permeability by altering the abundances of barrier-modulating metabolites.

We successfully established LGG mono-associated gnotobiotic mice fed by trp-sufficient (trp+) or trp-free (trp−) diets. We obtained a comprehensive untargeted metabolomic profile in intestinal lumen, serum, and liver, along with tissue-specific transcriptome in individual mice with or without LGG colonization. Our newly developed metabolome-transcriptome correlation analysis uncovered tissue-specific, LGG-regulated, dietary trp-dependent metabolites that either enhance or weaken gut barrier functions by interacting with the metabolites directly or indirectly via another LGG metabolite. The finding that LGG modulates barrier-regulating metabolites in dietary trp-dependent mechanism enhances our understanding of metabolite-based methods to enhance gut barrier function in millions of patients suspected of leaky gut.

### Definitions

The following are provided to facilitate the practice of the invention. They are not intended to limit the invention in any way.

For purposes of the present invention, “a” or “an” entity refers to one or more of that entity; for example, “a cDNA” refers to one or more cDNA or at least one cDNA. As such, the terms “a” or “an,” “one or more” and “at least one” can be used interchangeably herein. It is also noted that the terms “comprising,” “including,” and “having” can be used interchangeably. Furthermore, a compound “selected from the group consisting of” refers to one or more of the compounds in the list that follows, including mixtures (i.e. combinations) of two or more of the compounds. According to the present invention, an isolated, or biologically pure molecule is a compound that has been removed from its natural milieu. As such, “isolated” and “biologically pure” do not necessarily reflect the extent to which the compound has been purified. An isolated compound of the present invention can be obtained from its natural source, can be produced using laboratory synthetic techniques or can be produced by any such chemical synthetic route.

As used herein, the term “about” means a variability of 10% from the reference given, unless otherwise specified.

“Patient” or “subject” as used herein means a female mammalian animal, including a human, a veterinary or farm animal, a domestic animal or pet, and animals normally used for clinical research. In one embodiment, the subject of these methods and compositions is a human.

“Control” or “Control subject” as used herein refers to both an individual without an inflammatory disease or multiple individuals without the inflammatory disease or numerical or graphical averages of the expression levels of the selected biomarkers obtained from large groups of individuals without the inflammatory disease. In certain embodiments, the control subject is an individual without inflammatory bowel disease. In one embodiment, the IBD is ulcerative colitis and/or Crohn's Disease. Such controls are the types that are commonly used in similar diagnostic assays for other biomarkers. Selection of the particular class of controls depends upon the use to which the diagnostic methods and compositions are to be put by the physician. As used herein, the term “predetermined control” refers to a numerical level, average, mean or average range of the expression of a biomarker in a defined population. The predetermined control level is preferably provided by using the same assay technique as is used for measurement of the subject's biomarker levels, to avoid any error in standardization. In addition, a predetermined control may also be a negative predetermined control. The control can refer to a numerical average, mean or average range of the expression of one or more biomarkers, in a defined population, rather than a single subject.

Leaky gut syndrome, also called increased intestinal permeability, is a well-recognized and common diagnosis within the community of integrative doctors (B. Brom, South African Family Practice, 52, 314-316 (2010) Camilleri, M. Gut 68, 1516-1526, (2019)). Health care practitioners who diagnose this syndrome explain that intestinal inflammation widens the junctions between the cells of the intestinal lining. Increased permeability stimulates classic hypersensitivity responses to foods and to components of the normal gut flora. Bacterial endotoxins, cell wall polymers and dietary gluten may cause “non-specific” activation of inflammatory pathways (L. Galland, (1995), viewed 26 Oct. 2011, http://www.mdheal.org/leakygut.htm). Low grade fever, transient gut pain, and a sense of inability to absorb nutrients are some reported symptoms in otherwise undiagnosed patients. Leaky gut is often associated with various auto immune and inflammatory diseases (Mu, Q., Kirby, J., et al Front Immunol 8, 598, (2017) Kinashi, Y. & Hase, K. Frontiers in Immunology 12, doi: 10.3389/fimmu.2021.673708 (2021).

As used herein, the expression “inflammatory disease” is used herein in the broadest sense and includes all diseases and pathological conditions having etiologies associated with a systemic or local abnormal and/or uncontrolled inflammatory response. For instance, over-expression of proinflammatory cytokines without proper controls leads to a variety of inflammatory diseases and disorders. This term includes both acute inflammatory diseases and chronic inflammatory diseases.

In particular, the above-mentioned inflammatory diseases may be one or more selected from the group consisting of asthma, preperfusion injury, transplant rejection, sepsis, septic shock, arthritis, rheumatoid arthritis, acute arthritis, chronic rheumatoid arthritis, gouty arthritis, acute gouty arthritis, chronic inflammatory arthritis, degenerative arthritis, infectious arthritis, Lyme arthritis, proliferative arthritis, psoriatic arthritis, vertebral arthritis, and juvenile-onset rheumatoid arthritis, osteoarthritis, arthritis chronica progrediente, arthritis deformans, polyarthritis chronica primaria, reactive arthritis, and ankylosing spondylitis), x-linked hyper IgM syndrome, sclerosis, systemic sclerosis, multiple sclerosis (MS), spino-optical MS, primary progressive MS (PPMS), relapsing remitting MS (RRMS), progressive systemic sclerosis, atherosclerosis, arteriosclerosis, sclerosis disseminata, and ataxic sclerosis, inflammatory bowel disease (IBD), Crohn's disease, colitis, ulcerative colitis, colitis ulcerosa, microscopic colitis, collagenous colitis, colitis polyposa, necrotizing enterocolitis, transmural colitis, autoimmune inflammatory bowel disease, pyoderma gangrenosum, erythema nodosum, primary sclerosing cholangitis, episcleritis, respiratory distress syndrome, adult or acute respiratory distress syndrome (ARDS), meningitis, inflammation of all or part of the uvea, iritis, choroiditis, an autoimmune hematological disorder, rheumatoid spondylitis, sudden hearing loss, IgE-mediated diseases such as anaphylaxis and allergic and atopic rhinitis, encephalitis, Rasmussen's encephalitis, limbic and/or brainstem encephalitis, uveitis, anterior uveitis, acute anterior uveitis, granulomatous uveitis, nongranulomatous uveitis, phacoantigenic uveitis, posterior uveitis, autoimmune uveitis, glomerulonephritis (GN), idiopathic membranous GN or idiopathic membranous nephropathy, membrano- or membranous proliferative GN (A/IPGN), rapidly progressive GN, allergic conditions, autoimmune myocarditis, leukocyte adhesion deficiency, systemic lupus erythematosus (SLE) or systemic lupus erythematodes such as cutaneous SLE, subacute cutaneous lupus erythematosus, neonatal lupus syndrome (NLE), lupus erythematosus disseminatus, lupus (including nephritis, cerebritis, pediatric, non-renal, extra-renal, discoid, alopecia), juvenile onset (Type I) diabetes mellitus, including pediatric insulin-dependent diabetes mellitus (IDDM), adult onset diabetes mellitus (Type II diabetes), autoimmune diabetes, idiopathic diabetes insipidus, immune responses associated with acute and delayed hypersensitivity mediated by cytokines and T-lymphocytes, tuberculosis, sarcoidosis, granulomatosis, lymphomatoid granulomatosis, Wegener's granulomatosis, agranulocytosis, vasculitides, including vasculitis, large vessel vasculitis, polymyalgia rheumatica, giant cell (Takayasu's) arteritis, medium vessel vasculitis, Kawasaki's disease, polyarteritis nodosa, microscopic polyarteritis, CNS vasculitis, necrotizing, cutaneous, hypersensitivity vasculitis, systemic necrotizing vasculitis, and ANCA-associated vasculitis, such as Churg-Strauss vasculitis or syndrome (CSS), temporal arteritis, aplastic anemia, autoimmune aplastic anemia, Coombs positive anemia, Diamond Blackfan anemia, hemolytic anemia or immune hemolytic anemia including autoimmune hemolytic anemia (AIHA), pernicious anemia (anemia perniciosa), Addison's disease, pure red cell anemia or aplasia (PRCA), Factor VIII deficiency, hemophilia A, autoimmune neutropenia, pancytopenia, leukopenia, diseases involving leukocyte diapedesis, CNS inflammatory disorders, multiple organ injury syndrome such as those secondary to septicemia, trauma or hemorrhage, antigen-antibody complex-mediated diseases, anti-glomerular basement membrane disease, anti-phospholipid antibody syndrome, allergic neuritis, Bechet's or Behcet's disease, Castleman's syndrome, Goodpasture's syndrome, Reynaud's syndrome, Sjogren's syndrome, Stevens-Johnson syndrome, pemphigus, optionally pemphigus vulgaris, pemphigus foliaceus, pemphigus mucus-membrane pemphigoid, pemphigus erythematosus, autoimmune polyendocrinopathies, Reiter's disease or syndrome, immune complex nephritis, antibody-mediated nephritis, neuromyelitis optica, polyneuropathies, chronic neuropathy, IgM polyneuropathies, IgM-mediated neuropathy, thrombocytopenia, thrombotic thrombocytopenia purpura (TTP), idiopathic thrombocytopeniarpura (ITP), autoimmune orchitis and oophoritis, primary hypothyroidism, hypoparathyroidism, autoimmune thyroiditis, Hashimoto's disease, chronic thyroiditis (Hashimoto's thyroiditis); subacute thyroiditis, autoimmune thyroid disease, idiopathic hypothyroidism, Grave's disease, polyglandular syndromes such as autoimmune polyglandular syndromes (or polyglandular endocrinopathy syndromes), parancoplastic syndromes, including neurologic parancoplastic syndromes such as Lambert-Eaton myasthenic syndrome or Eaton-Lambert syndrome, stiff-man or stiff-person syndrome, encephalomyelitis, allergic encephalomyelitis, experimental allergic encephalomyelitis (EAE), myasthenia gravis, thymoma-associated myasthenia gravis, cerebellar degeneration, neuromyotonia, opsoclonus or opsoclonus myoclonus syndrome (OMS), and sensory neuropathy, multifocal motor neuropathy, Shechan's syndrome, autoimmune hepatitis, chronic hepatitis, lupoid hepatitis, giant cell hepatitis, chronic active hepatitis or autoimmune chronic active hepatitis, lymphoid interstitial pneumonitis, bronchiolitis obliterans (non-transplant) vs NSIP, Guillain-Barre syndrome, Berger's disease (IgA nephropathy), idiopathic IgA nephropathy, linear IgA dermatosis, primary biliary cirrhosis, pneumonocirrhosis, autoimmune enteropathy syndrome, Celiac disease, Coeliac disease, celiac sprue (gluten enteropathy), refractory sprue, idiopathic sprue, cryoglobulinemia, amylotrophic lateral sclerosis (ALS; Lou Gehrig's disease), coronary artery disease, autoimmune car disease such as autoimmune inner ear disease (AGED), autoimmune hearing loss, opsoclonus myoclonus syndrome (OMS), polychondritis such as refractory or relapsed polychondritis, pulmonary alveolar proteinosis, amyloidosis, scleritis, a non-cancerous lymphocytosis, a primary lymphocytosis, which includes monoclonal B cell lymphocytosis, optionally benign monoclonal gammopathy or monoclonal garnmopathy of undetermined significance, MGUS, peripheral neuropathy, parancoplastic syndrome, channelopathies such as epilepsy, migraine, arrhythmia, muscular disorders, deafness, blindness, periodic paralysis, and channelopathies of the CNS, autism, inflammatory myopathy, focal segmental glomerulosclerosis (FSGS), endocrine opthalmopathy, uvcoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases, diabetic nephropathy, Dressler's syndrome, alopecia greata, CREST syndrome (calcinosis, Raynaud's phenomenon, esophageal dysmotility, sclerodactyl), and telangiectasia), male and female autoimmune infertility, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, leishmaniasis, kypanosomiasis, schistosomiasis, ascariasis, aspergillo sis, Sampter's syndrome, Caplan's syndrome, dengue, endocarditis, endomyocardial fibrosis, diffuse interstitial pulmonary fibrosis, interstitial lung fibrosis, idiopathic pulmonary fibrosis, cystic fibrosis, endophthalmitis, erythema elevatum et diutinum, erythroblastosis fetalis, cosinophilic faciitis, Shulman's syndrome, Felty's syndrome, flariasis, cyclitis such as chronic cyclitis, heterochronic cyclitis, iridocyclitis, or Fuch's cyclitis, Henoch-Schonlein purpura, human immunodeficiency virus (HIV) infection, echovirus infection, cardiomyopathy, Alzheimer's disease, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, giant cell polymyalgia, endocrine ophthamopathy, chronic hypersensitivity pneumonitis, keratoconjunctivitis sicca, epidemic keratoconjunctivitis, idiopathic nephritic syndrome, minimal change nephropathy, benign familial and ischemia-reperfusion injury, retinal autoimmunity, joint inflammation, bronchitis, chronic obstructive airway disease, silicosis, aphthae, aphthous stomatitis, arteriosclerotic disorders, aspermiogenesc, autoimmune hemolysis, Boeck's disease, cryoglobulinemia. Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis (e.g. chronic pancreatitis), polyradiculitis acuta, pyoderma gangrenosum, Quervain's thyreoiditis, acquired splenic atrophy, infertility due to antispermatozoan antibodies, non-malignant thymoma, vitiligo, SCID and Epstein-Barr virus-associated diseases, acquired immune deficiency syndrome (AIDS), parasitic diseases such as Lesihmania, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, peripheral neuropathy, autoimmune polyglandular syndrome type I, adult-onset idiopathic hypoparathyroidism (AOIH), alopecia totalis, dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, an cosinophil-related disorder such as cosinophilia, pulmonary infiltration cosinophilia, cosinophilia-myalgia syndrome, Loffler's syndrome, chronic cosinophilic pneumonia, tropical pulmonary cosinophilia, bronchopneumonic aspergillosis, aspergilloma, or granulomas containing eosinophils, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia, autoimmune disorders associated with collagen disease, rheumatism, neurological disease, ischemic re-perfusion disorder, reduction in blood pressure response, vascular dysfunction, antgiectasis, tissue injury, cardiovascular ischemia, hyperalgesia, cerebral ischemia, and disease accompanying vascularization, allergic hypersensitivity disorders, glomerulonephritides, reperfusion injury, reperfusion injury of myocardial or other tissues, dermatoses with acute inflammatory components, acute purulent meningitis or other central nervous system inflammatory disorders, ocular and orbital inflammatory disorders, granulocyte transfusion-associated syndromes, cytokine-induced toxicity, acute serious inflammation, chronic intractable inflammation, pyelitis, pneumonocirrhosis, diabetic retinopathy, diabetic large-artery disorder, endarterial hyperplasia, peptic ulcer, valvulitis, nonalcoholic fatty liver disease and endometriosis.

Inflammatory bowel disease is a group of gastrointestinal diseases characterized by chronic and periodic inflammation. The major forms of inflammatory bowel disease are Crohn's disease and ulcerative colitis, although other forms of inflammatory bowel disease include collagenous colitis, lymphocytic colitis, ischaemic colitis, diversion colitis, Behcet's disease and indeterminate colitis. With the scale and severity of such disorders worldwide it is therefore advantageous to provide a composition to treat or prevent inflammatory disorders of the digestive tract such as ileitis, colitis, rectal inflammation, pharyngitis, leaky gut syndrome, irritable bowel syndrome and/or inflammatory bowel disease. The phrase “inflammatory bowel disease or IBD” refers to primarily ulcerative colitis (UC) and Crohn's (CD). These are chronic conditions of uncertain etiology, characterized by recurrent episodes of abdominal pain, often with diarrhea. Although both ulcerative colitis and Crohn's disease have distinct pathologic findings, a significant percentage of patients with inflammatory bowel disease (IBD) have indeterminate findings. Crohn's disease is also referred to a regional enteritis, terminal ileitis, or granulomatous ileocolitis.

An “autoimmune disease” is a condition in which the body's immune system mistakes its own healthy tissues as foreign and attacks them. Most autoimmune diseases cause inflammation that can affect many parts of the body. The parts of the body affected depend on which autoimmune disease a person has. Common signs and symptoms include fatigue, fever, muscle aches, joint pain and swelling, skin problems, abdominal pain, digestion problems, and swollen glands. The symptoms often come and go and can be mild or severe. There are many different types of autoimmune diseases. They are more common in women and can run in families. Also called autoimmune condition. Exemplary autoimmune diseases include, without limitation, systemic lupus erythematosus, diabetes, rheumatoid arthritis, reactive arthritis, multiple sclerosis, pemphigus vulgaris, celiac disease, Crohn's disease, inflammatory bowel disease, ulcerative colitis, autoimmune thyroid disease.

The phrase “Type 1 diabetes (TID)” refers to a chronic (lifelong) disease that occurs when the pancreas produces too little insulin to regulate blood sugar levels appropriately. TID, often called juvenile or insulin-dependent diabetes results from altered metabolism of carbohydrates (including sugars such as glucose), proteins, and fats. In type 1 diabetes, the beta cells of the pancreas produce little or no insulin, the hormone that allows glucose to enter body cells. Once glucose enters a cell, it is used as fuel. Without adequate insulin, glucose builds up in the bloodstream instead of going into the cells. The body is unable to use this glucose for energy despite high levels in the bloodstream, leading to increased hunger. In addition, the high levels of glucose in the blood cause the patient to urinate more, which in turn causes excessive thirst. Within 5 to 10 years after diagnosis, the insulin-producing beta cells of the pancreas are completely destroyed, and no more insulin is produced.

“Sample” as used herein means any biological fluid or tissue that contains the biomarkers. The most suitable samples for use in the methods and with the compositions are blood samples, including serum, plasma, whole blood, and peripheral blood. It is also anticipated that other biological fluids, such as saliva or urine, vaginal or cervical secretions, amniotic fluid, and placental fluid may be used similarly. Such samples may further be diluted with saline, buffer or a physiologically acceptable diluent. Alternatively, such samples are concentrated by conventional means.

By “change in expression” is meant an increased expression level of a selected biomarker, or upregulation of the genes or transcript encoding it in comparison to the reference or control; a decreased expression level of a selected biomarker or a downregulation of the genes or transcript encoding it in comparison to the reference or control; or a combination of certain increased/upregulated and decreased/down regulated biomarkers. The degree of change in target expression can vary with each individual and is subject to variation with each population. For example, in one embodiment, a large change, e.g., 2-3 fold increase or decrease in a small number of biomarkers, e.g., from 1 to 9 characteristic biomarkers, is statistically significant. In another embodiment, a smaller relative change in about 5, 10, 15, 20, 25, or more biomarkers is statistically significant.

By “target biomarker” or “target biomarker signature” as used herein is meant those proteins/peptides or the genes/transcripts encoding same, the expression of which changes (either in an up-regulated or down-regulated manner) characteristically in the presence of an inflammatory disease or inflammatory bowel disease from that in a healthy individual. In one embodiment, at least one target biomarker forms a suitable biomarker signature for use in the methods and compositions. In one embodiment, at least two target biomarkers form a suitable biomarker signature for use in the methods and compositions. Specific biomarker signatures can include any combination of inflammatory biomarkers employing at least one biomarker identified herein. One skilled in the art may readily reproduce the compositions and methods described herein by use of the sequences of the biomarkers, all of which are publicly available from conventional sources, such as GenBank.

The term “microarray” refers to an ordered arrangement of hybridizable array elements. e.g., primers, probes, ligands, on a substrate.

The term “ligand” refers to a molecule that binds to an expression product, protein or peptide, and includes antibodies and fragments thereof.

The term “polynucleotide,” when used in singular or plural form, generally refers to any polyribonucleotide or polydeoxyribonucleotide, which may be unmodified RNA or DNA or modified RNA or DNA, respectively. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The term “polynucleotide” specifically includes cDNAs. The term includes DNAs (including cDNAs) and RNAs that contain one or more modified bases. In general, the term “polynucleotide” embraces all chemically, enzymatically and/or metabolically modified forms of unmodified polynucleotides, as well as the chemical forms of DNA and RNA characteristic of viruses and cells, including simple and complex cells.

The term “oligonucleotide” refers to a relatively short polynucleotide of less than 20 bases, including, without limitation, single-stranded deoxyribonucleotides, single- or double-stranded ribonucleotides, RNA:DNA hybrids and double-stranded DNAs. Oligonucleotides, such as single-stranded DNA probe oligonucleotides, are often synthesized by chemical methods, for example using automated oligonucleotide synthesizers that are commercially available. However, oligonucleotides can be made by a variety of other methods, including in vitro recombinant DNA-mediated techniques and by expression of DNAs in cells and organisms.

As used herein, “labels” or “reporter molecules” are chemical or biochemical moieties useful for labeling a nucleic acid (including a single nucleotide), polynucleotide, oligonucleotide, or protein ligand, e.g., amino acid, peptide sequence, protein, or antibody. “Labels” and “reporter molecules” include fluorescent agents, chemiluminescent agents, chromogenic agents, quenching agents, radionucleotides, enzymes, substrates, cofactors, inhibitors, radioactive isotopes, magnetic particles, and other moieties known in the art. “Labels” or “reporter molecules” are capable of generating a measurable signal and may be covalently or noncovalently joined to an oligonucleotide or nucleotide (e.g., a non-natural nucleotide) or ligand.

It should be understood that while various embodiments in the specification are presented using “comprising” language, under various circumstances, a related embodiment is also described using “consisting of” or “consisting essentially of” language. It is to be noted that the term “a” or “an”, refers to one or more, for example, “an immunoglobulin molecule,” is understood to represent one or more immunoglobulin molecules. As such, the terms “a” (or “an”), “one or more,” and “at least one” is used interchangeably herein.

Unless defined otherwise in this specification, technical and scientific terms used herein have the same meaning as commonly understood by one of ordinary skill in the art and by reference to published texts, which provide one skilled in the art with a general guide to many of the terms used in the present application.

### Useful Target Biomarkers for Detecting Inflammatory Bowel Disease

The “targets” of the compositions and methods of these inventions include, in one aspect, the genes, gene fragments, transcripts and the expression products, including the proteins in the urea cycle and/or the arginine-citrulline cycle, including without limitation, ASA, ASS1, ASL, and NOS2. As described in the Examples below, the inventors identified proteins that differed in expression between healthy patients and patients with an inflammatory disease such as IBD. Further analysis resulted in the identification of highly significant protein biomarkers that can reliably distinguish between healthy individuals and patients with an inflammatory disease such as IBD. In certain embodiments, compositions and methods for detecting patients with an inflammatory disease such as IBD utilize at least one of the biomarkers, or one of the specifically identified isoforms or fragments of known markers. In other embodiments, compositions and methods for detecting patients with an inflammatory disease such as IBD utilize at least two or more of the specific target biomarker protein forms identified herein.

In certain embodiments, a target for use herein is selected from fecal and/or serum metabolites correlated with ileum occludin. In certain embodiments, the target is selected from metabolites with a negative correlation with ileum occludin. In certain embodiments, said negatively correlated metabolites are selected from the metabolites identified in Table 1 have a negative person correlation (Pearson corr). Correlations are considered significant if the FDR is <0.05. In certain embodiments the negatively correlated metabolites include deoxyguanosine, uridine, thymidine, O-propanoylcamitine, O-butanoylcamitine, inosine, 5-methylthioadenosine, guanosine, sorbitol, L-argino-succinate, Glycerol-3-phosphate, Taurine, 2-phenylglycine, citrate, succinate, creatinine, malate, acetyllysine, ribitol, phenyllactic acid, uracil, uric acid, leucic acid, and 2-hydroxy-3-methylbutyric acid. These metabolites are highlighted in grey in Table 1.

TABLE 1A

| | | LGG trp− vs LGG trp+ | |
|---|---|---|---|
| Metabolites associated with Ileum occludin | | Pearson corr | FDR |
| Fecal Pos | Indole-3-acetamide | 0.9785 | 4.72E−05 |
| Fecal Pos | 5-hydroxy-L-tryptophan | 0.9683 | 1.04E−04 |
| Fecal Pos | Salicylamide | 0.9585 | 2.03E−04 |
| Fecal Pos | Methionine | 0.9502 | 1.79E−03 |
| Fecal Pos | o-acetyl-L-serine | 0.9483 | 7.04E−04 |
| Fecal Pos | Acetyllysine | 0.9327 | 1.08E−03 |
| Fecal Pos | Phenylalanine | 0.9299 | 1.04E−02 |
| Fecal Pos | Histidine | 0.9260 | 2.48E−03 |
| Fecal Pos | L-Octanoylcarnitine | 0.9230 | 2.94E−03 |
| Fecal Pos | Isoleucine | 0.9213 | 1.15E−02 |
| Fecal Pos | Lysine | 0.9199 | 1.78E−03 |
| Fecal Pos | Glycerophosphocholine | 0.9197 | 1.48E−03 |
| Fecal Neg | Glutaconic.acid | 0.9172 | 1.61E−03 |
| Fecal Neg | Acetyl.glycine | 0.9039 | 3.37E−03 |
| Fecal Pos | N-acetyl-glutamine | 0.8975 | 1.11E−02 |
| Fecl Neg | Biotin | 0.8868 | 6.56E−03 |
| Fecl Neg | Cytidine | 0.8859 | 7.35E−03 |
| Fecl Neg | Indole-3-carboxaldehyde | 0.8825 | 1.33E−02 |
| Fecal Pos | Leucine | 0.8756 | 5.39E−03 |
| Fecal Pos | Thiamine | 0.8717 | 5.52E−03 |
| Fecal Pos | Cytosine | 0.8690 | 7.96E−03 |
| Fecal Neg | Xanthine | 0.8636 | 4.42E−03 |
| Fecal Pos | O-Propanoylcarnitine | 0.8561 | 4.72E−03 |
| Fecal Pos | Serotonin | 0.8549 | 5.39E−03 |
| Fecal Neg | Sorbitol | 0.8538 | 7.72E−03 |
| Fecal Neg | Uric.acid | 0.8504 | 6.56E−03 |
| Fecal Neg | 2-Ketoisovalerate | 0.8461 | 1.41E−02 |
| Fecal Pos | Arginine | 0.8448 | 1.38E−02 |
| Fecal Pos | O-Decanoyl-L-carnitine | 0.8438 | 1.15E−02 |
| Fecal Neg | Ribonolactone | 0.8435 | 5.56E−03 |
| Fecal Neg | Aspartate | 0.8387 | 8.73E−03 |
| Fecal Neg | N-acetyl-glutamate | 0.8351 | 9.49E−03 |
| Fecal Pos | Nicotinamide.riboside | 0.8333 | 9.89E−03 |
| Fecal Pos | N-acetyl-L-ornithine | 0.8323 | 1.01E−02 |
| Fecal Neg | Glucuronic.acid | 0.8243 | 1.08E−02 |
| Fecal Pos | Tryptophan | 0.8222 | 1.42E−02 |
| Fecal Pos | Tyrosine | 0.8197 | 1.33E−02 |
| Fecal Neg | Undecanoic.acid | 0.7940 | 2.20E−02 |
| Fecal Neg | 2-Hydroxyglutarate | 0.7835 | 2.94E−02 |
| Fecal Pos | Spermine | 0.7830 | 2.22E−02 |
| Fecal Neg | Hypoxanthine | 0.7629 | 2.94E−02 |
| Fecal Pos | 2-phenylglycine | −0.9165 | 1.35E−03 |
| Fecal Neg | Citrate | −0.9325 | 7.80E−04 |
| | | LGG trp+ vs PBS trp+ | |
| Metabolites associated with Ileum occludin | | Pearson corr | FDR |
| Fecal Neg | 5-Hydroxytryptophan | 0.9290 | 8.66E−04 |
| Fecal Pos | 5-hydroxy-L-tryptophan | 0.9267 | 8.46E−04 |
| Fecal Pos | Indole-3-acetamide | 0.9070 | 2.81E−03 |
| Fecal Neg | N-acetyl-glutamate | 0.9009 | 1.68E−03 |
| Fecal Neg | N-acetyl-glutamine | 0.8923 | 2.32E−03 |
| Fecal Neg | Ribitol | 0.8873 | 3.69E−03 |
| Fecal Pos | Salicylamide | 0.8848 | 2.40E−03 |
| Fecal Pos | NG-dimethyl-L-arginine | 0.8780 | 4.00E−03 |
| Fecal Pos | Nicotinamide.riboside | 0.8756 | 4.89E−03 |
| Fecal Neg | o-acetyl-L-serine | 0.8610 | 1.12E−02 |
| Fecal Pos | Carnitine | 0.8431 | 8.83E−03 |
| Fecal Pos | Glutamate | 0.8102 | 1.81E−02 |
| Fecal Neg | Hypoxanthine | 0.8086 | 1.31E−02 |
| Fecal Neg | Methylphenyllactate | 0.7720 | 3.17E−02 |
| Fecal Pos | Histidine | 0.7642 | 3.38E−02 |
| Fecal Neg | Deoxyguanosine | −0.7220 | 4.83E−02 |
| Fecal Neg | Uridine | −0.7447 | 4.05E−02 |
| Fecal Neg | Thymidine | −0.8304 | 6.92E−03 |
| Fecal Pos | O-Propanoylcarnitine | −0.8321 | 1.27E−02 |
| Fecal Pos | O-Butanoylcarnitine | −0.8421 | 1.01E−02 |
| Fecal Pos | Inosine | −0.8722 | 5.98E−03 |
| Fecal Pos | 5-.Methylthioadenosine | −0.8730 | 7.06E−03 |
| Fecal Pos | Guanosine | −0.8829 | 2.81E−03 |
| Fecal Neg | Sorbitol | −0.9487 | 2.56E−04 |

TABLE 1B

| | | LGG trp− vs LGG trp+ | |
|---|---|---|---|
| Metabolites associated with Ileum occludin | | Pearson corr | FDR |
| Serum Neg | Indole-3-propionic.acid | 0.9754 | 1.07E−04 |
| Serum Neg | Tryptophan | 0.9610 | 2.25E−04 |
| Serum Pos | Indole-3-acetonitrile | 0.9414 | 8.83E−04 |
| Serum Pos | Indole-3-acetamide | 0.9097 | 4.80E−03 |
| Serum Pos | 5-Methoxytryptophan | 0.8843 | 5.85E−03 |
| Serum Pos | Serotonin | 0.8655 | 6.09E−03 |
| Serum Neg | Indoleacrylic.acid | 0.8647 | 1.40E−02 |
| Serum Neg | Indolelactic.acid | 0.8541 | 1.75E−02 |
| Serum Pos | Kynurenine | 0.8327 | 3.07E−02 |
| Serum Neg | Indole-3-carboxaldehyde | 0.8288 | 2.77E−02 |
| Serum Pos | Carnosine | 0.8216 | 8.62E−03 |
| Serum Neg | Hydroxyproline | 0.7880 | 3.05E−02 |
| Serum Pos | Indole-3-carboxylic.acid | 0.7698 | 2.37E−02 |
| Serum Pos | L-arginino-succinate | −0.7654 | 2.22E−02 |
| Serum Neg | Succinate | −0.7684 | 4.29E−02 |
| Serum Neg | Creatinine | −0.7983 | 3.31E−02 |
| Serum Neg | Malate | −0.8069 | 2.73E−02 |
| Serum Pos | Acetyllysine | −0.8153 | 2.25E−02 |
| Serum Neg | Ribitol | −0.8273 | 2.02E−02 |
| Serum Neg | Phenyllactic.acid | −0.8334 | 1.63E−02 |
| Serum Neg | Uracil | −0.8984 | 2.47E−03 |
| Serum Neg | Uric.acid | −0.9108 | 2.57E−03 |
| Serum Neg | Leucic.acid | −0.9177 | 1.64E−03 |
| Serum Neg | 2-Hydroxy-3-methylbutyric.acid | −0.9422 | 4.08E−04 |
| | | LGG trp+ vs PBS trp+ | |
| Metabolites associated with Ileum occludin | | Pearson corr | FDR |
| Serum Pos | Methionine | 0.8761 | 4.01E−03 |
| Serum Pos | Methylnicotinamide | 0.8601 | 5.78E−03 |
| Serum Neg | Methylphenyllactate | 0.8345 | 8.03E−03 |
| Serum Neg | Indole-3-propionic.acid | 0.7874 | 3.43E−02 |
| Serum Pos | Adenosine | 0.7450 | 3.02E−02 |
| Serum Pos | L-arginino-succinate | −0.7405 | 2.57E−02 |
| Serum Neg | Glycerol-3-phosphate | −0.7750 | 2.18E−02 |
| Serum Pos | Taurine | −0.8087 | 2.45E−02 |

In certain embodiments, a target for use herein is ASA, known as L-argininosuccinic acid. It is a nonproteinogenic basic amino acid that is an important metabolic intermediate in the arginine-urea-nitric oxide cycle. The structure and chemical properties of ASA are publicly available, see, e.g. CAS Number 2387-71-5 and PubChem CID 16950.

In another embodiment a target for use herein is ASS1, known as argininosuccinate synthase 1. The amino acid sequence and nucleic acid sequence for ASS1 are publicly available, see, e.g., GENBANK Accession No. NG_011542.1

In another embodiment a target for use herein is ASL, known as argininosuccinate lyase. The amino acid sequence and nucleic acid sequence for ASL are publicly available, see, e.g., GENBANK Accession No. NG_009288.1

In another embodiment a target for use herein is NOS2, known as Nitric oxide synthase. The amino acid sequence and nucleic acid sequence for NOS2 are publicly available, see, e.g., GENBANK Accession No. NG_011470.1.

In still other embodiments, the target for use in the methods and compositions described herein can include various combinations of these target biomarkers and/or fragments thereof. In another embodiment a target combination, protein biomarker signature for use herein includes other known an inflammatory biomarkers in combination with the markers above.

### Diagnostic Reagents and Kits

### Labeled or Immobilized Biomarkers or Peptides

In one embodiment, diagnostic reagents for use in the methods of identifying a patient with an inflammatory disease such as IBD includes one target biomarker identified herein, associated with a detectable label or portion of a detectable label system. In another embodiment, a diagnostic reagent includes one target biomarker herein, immobilized on a substrate. In still another embodiment, combinations of such labeled or immobilized biomarkers are suitable reagents and components of a diagnostic kit. Among such immobilized or labeled biomarkers are those selected from the biomarkers:
i. L-argininosuccinic acid (ASA),
ii. argininosuccinate synthase 1 (ASS1),
iii. argininosuccinate lyase (ASL), and
iv. Nitric oxide synthase (NOS2).

In another aspect, suitable embodiments of such labeled or immobilized reagents include at least one, 2, 3, or all 4 of biomarkers (i) to (iv) or their unique peptide fragments therein.

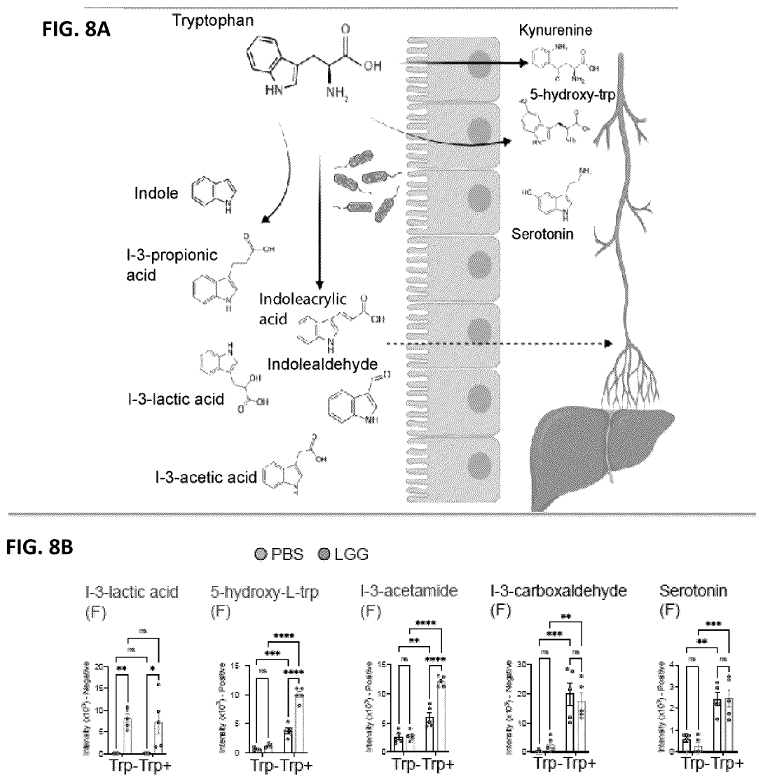

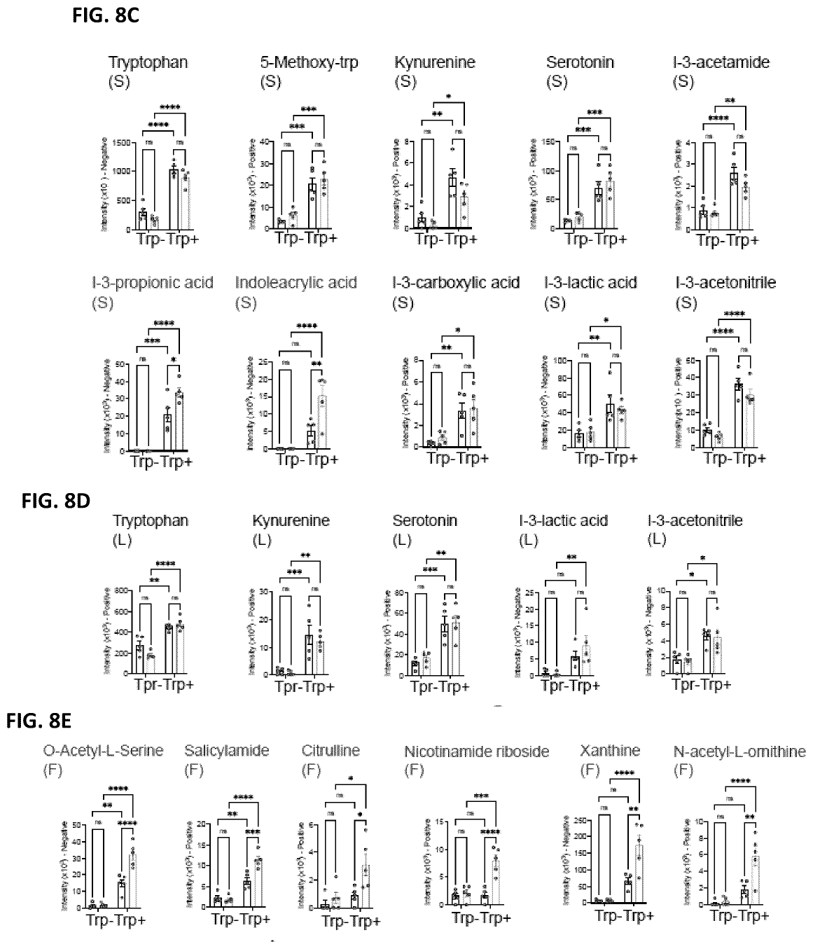

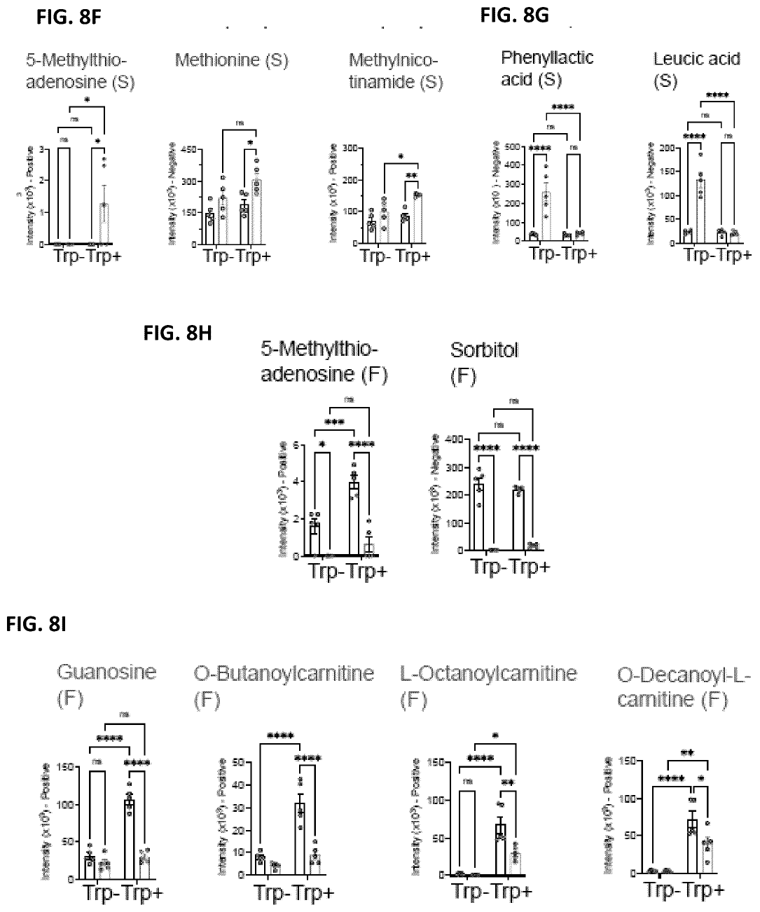

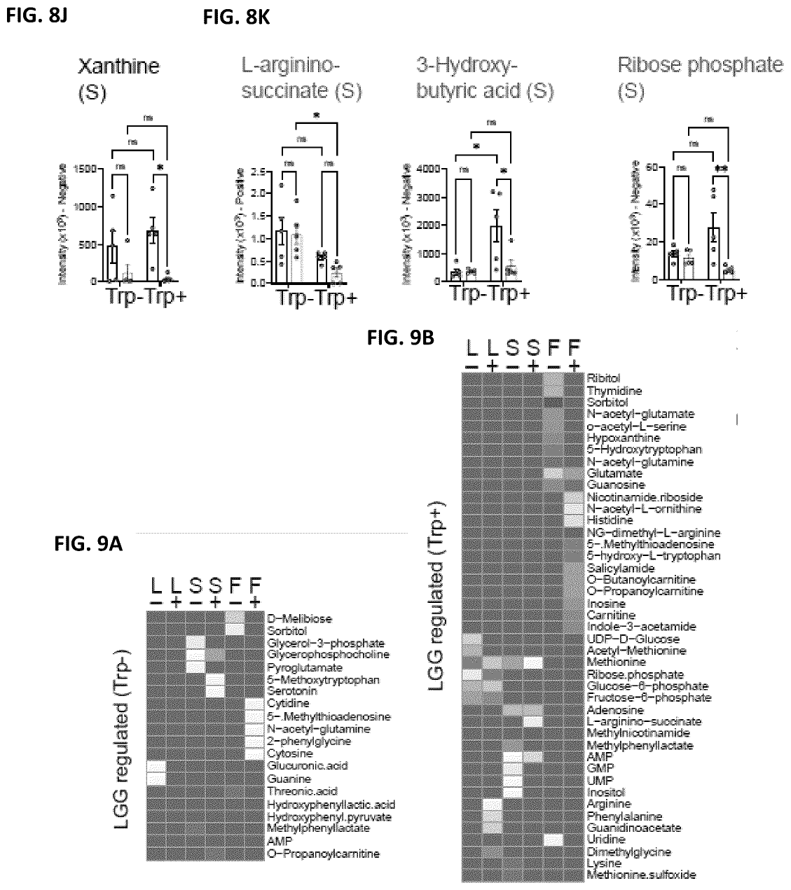

Any combination of labeled or immobilized biomarkers can be assembled in a diagnostic kit for the purposes of diagnosing an inflammatory disease such as IBD. For example, one embodiment of a diagnostic kit includes labeled or immobilized reagents (i) through (iii). Still other components of the biomarker signatures, associated with detectable labels or immobilized on substrates provide additional diagnostic kits. Still other components of the biomarker signatures are labeled or immobilized biomarkers or fragments thereof as listed in FIG. 8K, 9H, 14, or Tables 4A-4H.

For these reagents, the labels may be selected from among many known diagnostic labels, including those described above. Similarly, the substrates for immobilization may be any of the common substrates, glass, plastic, a microarray, a microfluidics card, a chip or a chamber.

Labeled or Immobilized Ligands that Bind the Biomarkers or Peptides

In another embodiment, the diagnostic reagent is a ligand that binds to a biomarker recited above or a unique peptide thereof. Such a ligand desirably binds to a protein biomarker or a unique peptide contained therein, and can be an antibody which specifically binds a single biomarker described above, or a unique peptide in that single biomarker. Various forms of antibody, e.g., polyclonal, monoclonal, recombinant, chimeric, as well as fragments and components (e.g., CDRs, single chain variable regions, etc.) may be used in place of antibodies. The ligand itself may be labeled or immobilized.

In another aspect, suitable embodiments of such labeled or immobilized reagents include at least one, 2, 3, 4 or more ligands. Each ligand binds to a single biomarker or their unique peptide fragments therein. In another aspect, other suitable embodiments of such labeled or immobilized reagents include an additional at least one, 2, or 3 ligands, wherein each ligand binds to a single biomarker or their unique peptide fragments therein.

Any combination of labeled or immobilized biomarker-binding ligands can be assembled in a diagnostic kit for the purposes of diagnosing an inflammatory disease such as IBD. For example, one embodiment of a diagnostic kit includes labeled or immobilized reagents that bind to biomarkers (i) through (iv).

Labeled or Immobilized Polynucleotide/Oligonucleotides that Hybridize to Genes, Gene Fragments, Gene Transcripts of Other Sequences Encoding the Biomarkers or Peptides

In another embodiment, the diagnostic reagent is a polynucleotide or oligonucleotide sequence that hybridizes to gene, gene fragment, gene transcript or nucleotide sequence encoding a biomarker of any one or more of the biomarkers described above or encoding a unique peptide thereof. Such a polynucleotide/oligonucleotide can be a probe or primer and may itself be labeled or immobilized. In another aspect, suitable embodiments of such labeled or immobilized reagents include at least one, 2, 3, 4, or more polynucleotide/oligonucleotide. Each polynucleotide/oligonucleotide hybridizes to a gene, gene fragment, gene transcript or expression product encoding a single biomarker or their unique peptide fragments therein. In another aspect, other suitable embodiments of such labeled or immobilized reagents include an additional at least one, 2, or 3 polynucleotide/oligonucleotides, wherein each sequence hybridizes to a gene, gene fragment, gene transcript of expression product encoding a single biomarker or their unique peptide fragments therein. In certain embodiments, the diagnostic reagent hybridizes to mRNA.

Any combination of labeled or immobilized biomarker-hybridizable sequences can be assembled in a diagnostic kit for the purposes of diagnosing an inflammatory disease such as IBD. For example, one embodiment of a diagnostic kit includes labeled or immobilized reagents that hybridize to at least one of the biomarkers described above. Another embodiment of a diagnostic kit includes labeled or immobilized reagents that hybridize all of the biomarkers described above. Still other components of the many biomarker signatures that may be formed by various combinations of polynucleotide/oligonucleotide sequences that hybridize to the biomarkers described above, or their unique fragments associated with detectable labels or immobilized on substrates provide additional diagnostic kits. In one embodiment, these polynucleotide or oligonucleotide reagent(s) are part of a primer-probe set, and the kit comprises both primer and probe. Each said primer-probe set amplifies a different gene, gene fragment or gene expression product that encodes a different biomarker of any combination of the markers described above, optionally including one or more additional biomarkers. In still another embodiment, additional polynucleotide or oligonucleotide sequences in the diagnostic reagent or kit, hybridize to a gene, gene fragment, gene transcript or expression product identified in FIG. 8K, 9H, 14, or Tables 4A-4H.

For use in the compositions the PCR primers and probes are preferably designed based upon the biomarker gene(s) to be amplified selected from the gene expression profile. The design of the primer and probe sequences is within the skill of the art once the particular gene target is selected. The particular methods selected for the primer and probe design and the particular primer and probe sequences are not limiting features of these compositions. A ready explanation of primer and probe design techniques available to those of skill in the art is summarized in U.S. Pat. No. 7,081,340, with reference to publicly available tools such as DNA BLAST software, the Repeat Masker program (Baylor College of Medicine), Primer Express (Applied Biosystems); MGB assay-by-design (Applied Biosystems); Primer3 (Steve Rozen and Helen J. Skaletsky (2000) Primer3 on the WWW for general users and for biologist programmers and other publications.

Thus, a composition for diagnosing an inflammatory disease such as IBD in a subject as described herein can be a kit containing multiple reagents or one or more individual reagents. For example, one embodiment of a composition includes a substrate upon which the biomarkers, polynucleotides or oligonucleotides, or ligands are immobilized. In another embodiment, the composition is a kit also contains optional detectable labels, immobilization substrates, optional substrates for enzymatic labels, as well as other laboratory items.

The compositions based on the biomarkers described herein, optionally associated with detectable labels, can be presented in the format of a microfluidics card, a chip or chamber, or a kit adapted for use with the assays described in the Examples, ELISAs or PCR, RT-PCR or Q PCR techniques described herein.

The selection of the ligands, poly/oligonucleotide sequences, their length, suitable labels and substrates used in the composition are routine determinations made by one of skill in the art in view of the teachings of which biomarkers form signature suitable for the diagnosis of an inflammatory disease such as IBD.

### Methods of Diagnosing an Inflammatory Disease

### Protein Assays

In one embodiment, a method for diagnosing an inflammatory disease such as IBD in a subject includes measuring in a biological fluid sample of the subject the expression level of an expression product, protein, or peptide fragment thereof selected from at least one biomarker described above. Alternatively, the method includes measuring a combination of two or more biomarkers described above. The method further involves comparing the subject's expression level of the selected biomarker or biomarker fragment with the level of the same expression product, protein, or peptide in the biological fluid of a reference or control subject. Changes in expression of the subject's selected biomarker from those of the reference or control correlates with a diagnosis of IBD.

In another embodiment, the above method further includes measuring in the biological fluid sample of the subject the expression level of an additional biomarker expression product, protein, or peptide fragment. In another embodiment, the above method further includes measuring in the biological fluid sample of the subject the expression level of two or more additional biomarker expression product, protein, or peptide fragments.

In another embodiment, the above method further includes measuring in the biological fluid sample of the subject the expression level of an additional biomarker expression product, protein, or peptide fragment of a biomarker identified in FIG. 8K, 9H, 14, or Tables 4A-4H.

In certain embodiments, the method of diagnosing an inflammatory disease such as IBD includes measuring in the biological fluid sample of the subject the expression level of a biomarker expression product, protein, or peptide fragment of a biomarker selected from:
i. L-argininosuccinic acid (ASA),
ii. argininosuccinate synthase 1 (ASS1),
iii. argininosuccinate lyase (ASL), and
iv. Nitric oxide synthase (NOS2).

In certain embodiments, the method includes measuring the expression level of at least one, 2, 3, or all 4 of biomarkers (i) to (iv) or their unique peptide fragments therein.

In this diagnostic method, a change in expression level of one or more of the selected biomarkers in comparison to the control reference may be an increase or decrease in the expression levels of the individual biomarkers. This method may employ any of the suitable diagnostic reagents or kits or compositions described above.

The measurement of the inflammatory biomarkers in the biological sample may employ any suitable ligand, e.g., antibody (or antibody to any second biomarker) to detect the inflammatory biomarker expression product or protein. Such antibodies may be presently extant in the art or presently used commercially, such as those available as part of commercial antibody ELISA assay kits or that may be developed by techniques now common in the field of immunology. As used herein, the term “antibody” refers to an intact immunoglobulin having two light and two heavy chains or any fragments thereof. Thus, a single isolated antibody or fragment may be a polyclonal antibody, a high affinity polyclonal antibody, a monoclonal antibody, a synthetic antibody, a recombinant antibody, a chimeric antibody, a humanized antibody, or a human antibody. The term “antibody fragment” refers to less than an intact antibody structure, including, without limitation, an isolated single antibody chain, a single chain Fv construct, a Fab construct, a light chain variable or complementarity determining region (CDR) sequence, etc. A recombinant molecule bearing the binding portion of an inflammatory biomarker antibody may also be used in a diagnostic assay. As used herein, the term “antibody” may also refer, where appropriate, to a mixture of different antibodies or antibody fragments that bind to the selected biomarker. Such different antibodies may bind to different biomarkers or different portions of the same inflammatory biomarker protein than the other antibodies in the mixture. Such differences in antibodies used in the assay may be reflected in the CDR sequences of the variable regions of the antibodies. Such differences may also be generated by the antibody backbone, for example, if the antibody itself is a non-human antibody containing a human CDR sequence, or a chimeric antibody or some other recombinant antibody fragment containing sequences from a non-human source. Antibodies or fragments useful in the method of this invention may be generated synthetically or recombinantly, using conventional techniques or may be isolated and purified from plasma or further manipulated to increase the binding affinity thereof. It should be understood that any antibody, antibody fragment, or mixture thereof that binds one of the biomarkers or a particular sequence of the selected inflammatory biomarkers may be employed in the methods of the present invention, regardless of how the antibody or mixture of antibodies was generated.

Similarly, the antibodies may be tagged or labeled with reagents capable of providing a detectable signal, depending upon the assay format employed. Such labels are capable, alone or in concert with other compositions or compounds, of providing a detectable signal. Where more than one antibody is employed in a diagnostic method, e.g., such as in a sandwich ELISA, the labels are desirably interactive to produce a detectable signal. Most desirably, the label is detectable visually, e.g., colorimetrically. A variety of enzyme systems operate to reveal a colorimetric signal in an assay, e.g., glucose oxidase (which uses glucose as a substrate) releases peroxide as a product that in the presence of peroxidase and a hydrogen donor such as tetramethyl benzidine (TMB) produces an oxidized TMB that is seen as a blue color. Other examples include horseradish peroxidase (HRP) or alkaline phosphatase (AP), and hexokinase in conjunction with glucose-6-phosphate dehydrogenase that reacts with ATP, glucose, and NAD+ to yield, among other products, NADH that is detected as increased absorbance at 340 nm wavelength.

Other label systems that may be utilized in the methods of this invention are detectable by other means, e.g., colored latex microparticles (Bangs Laboratories, Indiana) in which a dye is embedded may be used in place of enzymes to provide a visual signal indicative of the presence of the resulting selected biomarker-antibody complex in applicable assays. Still other labels include fluorescent compounds, radioactive compounds or elements. Preferably, an anti-biomarker antibody is associated with, or conjugated to a fluorescent detectable fluorochromes, e.g., fluorescein isothiocyanate (FITC), phycoerythrin (PE), allophycocyanin (APC), coriphosphine-O (CPO) or tandem dyes, PE-cyanin-5 (PC5), and PE-Texas Red (ECD). Commonly used fluorochromes include fluorescein isothiocyanate (FITC), phycoerythrin (PE), allophycocyanin (APC), and also include the tandem dyes, PE-cyanin-5 (PC5), PE-cyanin-7 (PC7), PE-cyanin-5.5, PE-Texas Red (ECD), rhodamine, PerCP, fluorescein isothiocyanate (FITC) and Alexa dyes. Combinations of such labels, such as Texas Red and rhodamine, FITC+PE, FITC+PECy5 and PE+PECy7, among others may be used depending upon assay method.

In certain embodiments, the biomarkers identified herein can be converted to another metabolite which is oxidized with the conversion of a probe into a highly colored and/or fluorescent species proportional to the amount of the biomarker in the sample. In certain embodiments, the levels of biomarker can be quantified in the range between 0.1-10 nmoles/well (2-200 μM)

Detectable labels for attachment to antibodies useful in diagnostic assays of this invention may be easily selected from among numerous compositions known and readily available to one skilled in the art of diagnostic assays. The inflammatory biomarker-antibodies or fragments useful in this invention are not limited by the particular detectable label or label system employed. Thus, selection and/or generation of suitable inflammatory biomarker antibodies with optional labels for use in this invention is within the skill of the art, provided with this specification, the documents incorporated herein, and the conventional teachings of immunology.

Similarly the particular assay format used to measure the selected inflammatory biomarker in a biological sample may be selected from among a wide range of immunoassays, such as enzyme-linked immunoassays, such as those described in the examples below, sandwich immunoassays, homogeneous assays, immunohistochemistry formats, or other conventional assay formats. One of skill in the art may readily select from any number of conventional immunoassay formats to perform this invention.

Employing ligand binding to the biomarker proteins or multiple biomarkers forming the signature enables more precise quantitative assays, as illustrated by the ELISA assays.

### Nucleic Acid Assays

Still other methods useful in performing the diagnostic steps described herein are known in the art. Such methods include methods based on hybridization analysis of polynucleotides, methods based on sequencing of polynucleotides, proteomics-based methods or immunochemistry techniques. The most commonly used methods known in the art for the quantification of mRNA expression in a sample include northern blotting and in situ hybridization; RNAse protection assays; and PCR-based methods, such as reverse transcription polymerase chain reaction (RT-PCR) or qPCR. Alternatively, antibodies may be employed that can recognize specific DNA-protein duplexes. The methods described herein are not limited by the particular techniques selected to perform them. Exemplary commercial products for generation of reagents or performance of assays include TRI-REAGENT, Qiagen RNeasy mini-columns, MASTERPURE Complete DNA and RNA Purification Kit (EPICENTRE®, Madison, Wis.), Paraffin Block RNA Isolation Kit (Ambion, Inc.) and RNA Stat-60 (Tel-Test), the MassARRAY-based method (Sequenom, Inc., San Diego, CA), differential display, amplified fragment length polymorphism (iAFLP), and BeadArray™ technology (Illumina, San Diego, CA) using the commercially available Luminex 100 LabMAP system and multiple color-coded microspheres (Luminex Corp., Austin, Tex.) and high coverage expression profiling (HiCEP) analysis.

Thus, in yet another embodiment, a method for diagnosing an inflammatory disease such as IBD in a subject involves measuring in a biological fluid sample of the subject the expression level of a gene, gene fragment, gene transcript (e.g., mRNA) or expression product encoding one or more of the biomarkers. Alternatively, the method includes measuring the expression level of a gene, gene fragment, gene transcript or expression product encoding a combination of two or more biomarkers. The method further includes comparing the subject's selected biomarker gene, gene fragment, gene transcript or expression product expression level with the level of the same gene, gene fragment, gene transcript or expression product in the biological fluid of a reference or control subject. Changes in expression of the subject's selected biomarker gene, gene fragment, gene transcript or expression products from those of the reference or control correlates with a diagnosis of the inflammatory disease.

In another embodiment, the above method further includes measuring in the biological fluid sample of the subject the expression level of an additional biomarker gene, gene fragment, gene transcript or expression product encoding fragment of a biomarker. In another embodiment, the above method further includes measuring in the biological fluid sample of the subject the expression level of two or more additional biomarker gene, gene fragment, gene transcript or expression product encoding biomarkers.

In another embodiment, the above method further includes measuring in the biological fluid sample of the subject the expression level of an additional biomarker gene, gene fragment, gene transcript or expression product encoding fragment of a biomarker identified in FIG. 8K, 9H, 14, or Tables 4A-4H.

In this diagnostic method, a change in expression level of one or more of the selected biomarker gene, gene fragment, gene transcript or expression product in comparison to the control reference may be an upregulation or down regulation in the expression of the individual biomarker gene, gene fragment, transcript or expression product. This method may employ any of the suitable diagnostic reagents or kits or compositions described above.

In yet another embodiment, the methods and compositions described herein may be used in conjunction with clinical risk factors to help physicians make more accurate decisions about how to manage patients with IBD.

### Methods of Monitoring Inflammatory Disease

In certain embodiments, provided are methods of monitoring the responsiveness to a treatment regimen in a subject being treated for an inflammatory disease such as inflammatory bowel disease (IBD). The methods comprise (a) obtaining a first sample from the subject being treated for IBD; (b) obtaining a second sample from the subject being treated; (c) measuring in the biological fluid sample of the subject the expression level of at least one biomarker provided herein; and (c) detecting a difference in the levels of the at least one biomarker between the two samples, wherein the difference indicates the responsiveness to the treatment regimen in the subject. By way of an example, a subject being treated will express certain levels of the biomarkers of the invention at the start of the treatment regimen. During the course of treatment, a sample or multiple samples can be obtained from the subject, and these samples can be used to determine a difference in levels of the biomarkers. In one embodiment, the pattern of the panel of biomarkers comprises gene expression levels of the biomarkers within the panel. Increased expression or decreased expression of the panel of biomarkers can indicate that the treatment regimen is successfully treating an inflammatory disease such as IBD. The expression level of a second set of biomarkers can also be used to indicate whether the treatment regimen is successful to treat the inflammatory disease such as IBD.

In certain embodiments, when determining a treatment regimen or monitoring the response to a treatment regimen, multiple samples can be obtained from the subject and the pattern of biomarkers can be determined for each sample that is obtained from the subject. Monitoring the levels of biomarkers over time and in response to the treatment regimen can provide one skilled in the art the information necessary to determine a treatment regimen, to maintain the same treatment regimen, or to change the treatment regimen.

The inflammatory bowel disease can, for example, be selected from ulcerative colitis or Crohn's disease. In certain embodiments, the inflammatory bowel disease is ulcerative colitis. In certain embodiments, the inflammatory bowel disease is Crohn's disease.

### Methods of Treating Leaky Gut

During leaky gut treatment, a therapeutic agent can reduce the inflammation that triggers the signs and symptoms, leading not only to symptom relief but also to long-term remission and reduced risks of complications. Treatment usually involves either drug therapy or surgery. The drugs for leaky gut therapy include, but are not limited to, nutraceuticals, anti-inflammatory drugs, antibiotics, immunomodulators, anti-diarrheal medications, pain relievers, iron supplements, and calcium and vitamin D supplements.

The drugs for leaky gut therapy can be administered through any conventional and pharmaceutically acceptable route. Conventional and pharmaceutically acceptable routes of administration include, but are not limited to, systemic routes, such as intraperitoneal, intravenous, intranasal, intravenous, intramuscular, intratracheal, and subcutaneous, administration. In one embodiment, the route of administration is oral. In another embodiment, the route of administration is intraperitoneal. In another embodiment, the route of administration is intravascular. Routes of administration may be combined, if desired. In some embodiments, the administration is repeated periodically.

As used herein, “treatment” refers to both therapeutic treatment and prophylactic or preventative measures, wherein the object is to prevent or slow down (lessen) the targeted pathologic condition or disorder. Those in need of treatment include those diagnosed with the disorder as well as those prone to have the disorder (e.g., a genetic predisposition) or those in whom the disorder is to be prevented. The terms “prevent.” “preventing.” and “prevention” refer to reducing the likelihood of the onset (or recurrence) of a disease, disorder, condition, or associated symptom(s).

The term “administering” with respect to the methods of the invention, means a method for therapeutically or prophylactically preventing, treating or ameliorating a syndrome, disorder or disease (e.g., an inflammatory bowel disease (IBD)) as described herein. Such methods include administering an effective amount of said therapeutic agent at different times during the course of a therapy or concurrently in a combination form. The methods of the invention are to be understood as embracing all known therapeutic treatment regimens.

The term “effective amount” or “therapeutically effective amount” means that amount of active compound or pharmaceutical agent, a combination of therapeutic compounds or pharmaceutical compositions thereof provided herein, that elicits the biological or medicinal response in a tissue system, animal or human, that is being sought by a researcher, veterinarian, medical doctor, or other clinician, which includes preventing, treating or ameliorating a syndrome, disorder, or disease being treated, or the symptoms of a syndrome, disorder or disease being treated (e.g., IBD).

As used herein. “a response” to a treatment in a subject diagnosed with an inflammatory bowel disease (IBD) can be a positive response or a negative response to the treatment. As used herein, a “positive response” to an IBD treatment refers to a response comprising at least one of mucosal healing, clinical response, and clinical remission resulting from the IBD treatment. As used herein, a “negative response” or “no response” to an IBD treatment refers to there is no response in any of mucosal healing, clinical response, and clinical remission resulting from the IBD treatment.

### Nutraceuticals

Nutraceuticals can be used in the treatment of an inflammatory disease such as IBD. Nutraceuticals can be defined as natural products that are used to supplement the diet by increasing the total dietary intake of important nutrients. This definition includes nutritional supplements such as vitamins, minerals, herbal extracts, antioxidants, amino acids, and protein supplements. Nutraceutical products fit into the newly created product category of “Dietary Supplements” as established by the F.D.A. in the Dietary Supplement Act of 1994. This act specifically defined dietary supplements to include: probiotics, prebiotics, digestive enzymes, vitamins, minerals, herbs or other botanicals, antioxidants, amino acids, or other dietary substances used to supplement the diet by increasing the total daily intake.

A “nutraceutical composition” is defined herein as a food composition fortified with ingredients capable of producing health benefits. Such a composition in the context of the present invention may also be indicated as foods for special dietary use; medical foods; and dietary supplements. For example, the food item or supplement may help to prevent or reduce symptoms associated with an inflammatory condition such as allergies (e.g. hay fever) and the like. As with the pharmaceutical composition, the amount of active ingredient in the food or food additive will depend on several factors. The food product will generally comprise a concentration that is sufficient to provide a consumer with an effective amount of active ingredient upon consumption of a regular (e.g. daily) portion of the food product. It will be recognized by those skilled in the art that the optimal quantity and spacing of individual dosages for achieving the therapeutic effects of the pharmaceutical composition, food item or food supplement described herein may easily be determined by the skilled person.

In certain embodiments, the nutraceutical compositions include Lactobacillus rhamnosus GG metabolites including the nutraceuticals identified in Tables 2 and 3. In certain embodiments, the nutraceutical compositions are administered orally and/or intravenously.

TABLE 2
Nutraceuticals for oral supplementation for leaky gut syndrome

| | LGG+ VS LGG− | |
|---|---|---|
| METABOLITE | R/FDR | LGG+ VS PBS+ R |
| Indole-3-acetamide | 0.98/0.000047 | 0.91/0.0028 |
| 5-hydroxy-L-tryptophan | 0.97/0.0001 | 0.93/0.00085 |
| Salicylamide | 0.96/0.0002 | 0.88/0.00024 |
| O-acetyl-L-serine | 0.95/0.0007 | 0.86/0.011 |
| Histidine | 0.93/0.0025 | 0.76/0.033 |
| N-acetyl-glutamine | 0.90/0.011 | 0.89/0.0023 |
| Acetyllysine | 0.93/0.0011 | — |
| NG-dimethyl-L-arginine | — | 0.88/0.004 |
| Nicotinamide riboside | 0.83/0.0098 | 0.88/0.0049 |

TABLE 3
Nutraceuticals for oral or IV supplementation
for leaky gut syndrome

| | LGG+ VS LGG− | |
|---|---|---|
| SERUM METABOLITE | R/FDR | LGG+ VS PBS+ R |
| Indole-3-propionic acid | 0.97/0.00011 | 0.79/0.03 |
| Indole-3-acetonitrile | 0.94/0.0008 | |
| 5-methoxytryptophan | 0.88/0.0058 | |
| serotonin | 0.87/0.006 | |
| Indoleacrylic acid | 0.86/0.014 | |
| Indolelactic acid | 0.85/0.017 | |
| kynurenine | 0.83/0.03 | |
| Indole-3-carboxyaldehyde | 0.83/0.027 | |
| methylnicotinamide | — | 0.86/0.0058 |

In certain embodiments, the nutraceutical compositions include metabolites correlated with ileum occludin. In certain embodiments, the compositions include metabolites with a positive correlation with ileum occludin. In certain embodiments, said positively correlated metabolites are selected from the metabolites identified in Table 1 with a positive Pearson correlation (Pearson Corr). Correlations are considered significant if the false discovery rate (FDR) is <0.05. In certain embodiments the positively correlated metabolites include 5-hydroxytryptophan, 5-hydroxy-L-tryptophan, indole-3-acetaminde, N-acetyl-glutamate, N-acetyl-glutamine, ribitol, salicylamide, NG-dimethyl-L-arginine, Nicotinamide, o-acetyl-L-serine, carnitine, glutamate, hypoxanthine, methylphenyllactate, histidine, methionine, methylnicotinamide. Methylphenyllactate, indole-3-propionic acid, adenosine, acetyllysine, phenylalanine, L-octanoylcarnitine, isoleucine, lysine, glycerophosphocholine, glutaconic acid, acetyl glycine, N-acetyl-glutamine, biotine, cytidine, indole-3-carboxaldehyde, leucine, thiamine, cytosine, xanthine, O-propanoylcarnitine, serotonin, uric acid, 2-ketoisovalerate, arginine, o-decanoyl-L-carnitine, ribonolactone, aspartate, n-acetyl-L-ornithine, glucuronic acid, tryptophan, tyrosine, undecanoic acid, 2-hydroxyglutarate, spermine, hypoanthine, indole-3-acetonitrile, indoleacrylic acid, indolelactic acid, kynurenine, hydroxproline, and indole-3-carboxylic acid.

### Anti-Inflammatory Drugs

Anti-inflammatory treatments are often the first step in the treatment of an inflammatory disease such as IBD. Anti-inflammatory drugs include, but are not limited to, aminosalicylates, corticosteroids, anti-tumor necrosis factor (TNF) agents, JAK inhibitors, anti-interleukin agents, and anti-integrin agents.

Examples of anti-TNF drugs include infliximab (Remicade), adalimumab (Humira), and golimumab (Simponi). JAK inhibitors can be the inhibitors against one or more of four JAK members: JAK1, Jak2, JAK3, and TYK2. Example of JAK inhibitors include filgotinib, peficitinib, tofacitinib (Xeljanz/Jakvinus), and upadacitinib. Anti-interleukin (IL) agents can be anti-IL-1, anti-IL-6, anti-IL-10, anti-IL-13, anti-IL-17, anti-IL-12/23, or anti-IL-23 agents. Anti-IL-12/23 agents are also called IL-12/23 blockade, including ustekinumab (Stelara). Examples of anti-IL-23 include BI 655066, briakinumab, guselkumab, tildrakizumab, and ustekinumab (Stelara). Examples of anti-integrin drugs include vedolizumab and natalizumab.

Aminosalicylates, given orally or rectally, can help control the inflammation of an inflammatory disease such as IBD by delivering a compound containing 5-aminosalicylic acid (5-ASA) to the bowel. Examples of aminosalicylates are sulfasalazine, mesalamine, olsalazine and balsalazide. These medications are used for both ulcerative colitis and Crohn's disease; however, they are much more effective for ulcerative colitis and are being used less often for Crohn's disease.

Corticosteroids are fast-acting anti-inflammatory drugs, which are used to treat acute (sudden onset and/or short duration) flare-ups. Because of their known side effects, doctors like to either avoid them completely or prescribe them for a short time. Corticosteroids can be given orally, rectally or intravenously. Examples of corticosteroids are prednisone, prednisolone, or methylprednisolone. Budesonide is a slightly different type of steroid, as very little gets absorbed into the body, so side effects are much less frequent.

### Antibiotics

Antibiotics, given orally or intravenously, are used selectively in patients with Crohn's disease and in patients with an inflammatory disease such as IBD who develop infection with Clostridium difficile. Examples of antibiotics are metronidazole and ciprofloxacin.

### Immunomodulators

It is believed that inflammatory diseases such as IBD is caused by an overactive immune system. Immunomodulators work by quieting down the immune system, helping to reduce inflammation. They can be given orally or by injection. Examples of immunomodulators are azathioprine (AZA), cyclosporine, 6-mercaptopurine (6 MP), and methotrexate (for Crohn's disease).

### Combination Therapies

One or more therapies included above as well as other therapies well known in the art can be used in combination to treat a patient with an inflammatory disease such as IBD. One or more therapies can be administered prior to, concurrently with, or subsequent to the administration of the other therapy described herein. Administration of one or more therapies and an additional therapy to a patient can occur simultaneously or sequentially by the same or different routes of administration. The suitability of a particular route of administration employed for a particular therapy will depend on the therapy itself. Routes of administration for the therapies for an inflammatory disease such as IBD are known to those of ordinary skill in the art.

In certain embodiments, the combination therapies described herein can be cyclically administered to a patient with an inflammatory disease such as IBD. Cycling therapy involves the administration of an active agent for a period of time, followed by a rest for a period of time, and repeating this sequential administration. Cycling therapy can reduce the development of resistance to one or more of the therapies, avoid or reduce the side effects of one of the therapies, and/or improves the efficacy of the treatment.

As used herein, the term “in combination” does not restrict the order in which therapies (e.g., prophylactic and/or therapeutic agents) are administered to a patient with an inflammatory disease such as IBD. In one embodiment, a first therapy is administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before) the administration of a second therapy provided herein. In one embodiment, a first therapy is administered concomitantly with the administration of a second therapy provided herein. In one embodiment, a first therapy is administered subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy provided herein.

Various therapies can be used in combination, including any of the exemplified therapies described above. Combination therapy can include two or more therapies selected from the group consisting of anti-inflammatory treatment, antibiotics, immunomodulators, anti-diarrheal medications, pain relievers, iron supplements, and calcium and vitamin D supplements.

Combination therapies can include, but is not limited to, for instance, administration of two or more anti-inflammatory drugs to the same subject, administration of one or more anti-inflammatory drug in combination with one or more antibiotic to the same subject, administration of one or more anti-inflammatory drug in combination with one or more immunomodulator to the same subject, administration of one or more immunomodulator in combination with one or more antibiotic to the same subject, and administration of one or more immunomodulators in combination with one or more antibiotics and one or more anti-inflammatory drugs to the same subject. Given the teachings and guidance provided herein one skilled in the art will understand that the disclosure herein is intended to include all combinations and permutations of two or more therapies. Thus, the various combinations and permutations set forth herein is intended to be exemplary and not limiting.

Based on the teachings and disclosures provided herein, one with ordinary skill in the art would be able to make and use various combination therapies with different agents disclosed herein and others known in the art that target one or more of the disclosed pathways.

### Materials and Methods

The following materials and methods are provided to facilitate the practice of the present invention.

### Mice

We had three series of studies: a gnotobiotic experiment involving mice monocolonized with LGG then fed trp-sufficient (0.2%, Cat. No. A10021BSu-1.5V) or -deficient (0%, Cat. No. A10033-1.5V) double-irradiated purified diets (Research Diets, New Brunswick, NJ), specific pathogen free (SPF) mice perfused with LGG, and SPF mice challenged with DSS then treated with LGG metabolites. C57BL/6, 2 months old, germ-free (GF) mice were obtained from Charles Rivers Laboratories (Wilmington, MA, USA). Upon delivery, GF female mice were housed in autoclaved gnotobiotic cages, 4 cages (n=5 mice per cage), on ventilated racks (Allentown, PA) at a temperature-controlled gnotobiotic facility at Rutgers New Jersey Medical School, with free access to autoclaved water. For the two other studies, C57BL/6, 2-3 months old female mice were obtained from Taconic Laboratories (Hudson, NY). All mice (˜20 g) were maintained on a 12:12 light-dark cycle. Prior to switching to purified diets, GF mice were fed an autoclaved and irradiated commercial semi-purified chow diet (0.2% trp. JL 5KA1, LabDiet®, Richmond, IN) while SPF mice were fed rodent diets (Cat. No. 5010, LabDiet®). Animal procedures and protocols were conducted in accordance with the Institutional Animal Care and Use Committee of Rutgers University.

### Experimental Designs

Gnotobiotic study. After 5 days of acclimatization under GF conditions, two cages of GF mice (n=10) were fed with a trp-free diet and another 2 cages with a trp-sufficient diet. Purified diets were sterilized by double-irradiation (Research Diets, New Brunswick, NJ) then stored in −80° C. Two weeks afterward, five mice from the trp-deficient and five from trp-sufficient were gavaged with LGG and the remaining mice with sterile PBS. Body weight and food intake were recorded weekly. Fecal pellets were collected aseptically one week before, then one and three weeks post inoculation, then stored (−80° C.) for later analysis. Feces were also collected twice a week then analyzed immediately to monitor gnotobiotic status. All mice were sacrificed three weeks post inoculation, then serum, liver, ileum and colon collected.

Perfusion study. Experiments were always paired (one mouse perfused with LGG, the other with Krebs-Ringer Buffer (KRB)). Under euthanasia, a transverse incision was made into the abdominal wall, and the small intestine with intact nerves and blood vessels were exposed. Incisions were made at the proximal (4 cm distal from the stomach) and medial sections of the small intestine, roughly 10 cm from the proximal incision. A gavage needle was inserted and secured to the proximal incision site and the lumen of the small intestine was then flushed with KRB (37° C.). A second gavage needle was then inserted into the distal incision and secured. A PE tube (Tygon, Saint-Gobain, PA) was attached to the superior gavage needle and perfusate was then administered for 4 h in a proximal to distal direction at a rate of 15 mL/hour using a multi-syringe infusion pump (Model 22 Multi-Syringe Pump: MA1 55-5920, Harvard Apparatus). Body temperatures were maintained at 37° C. using a closed loop homocothermic monitoring system (Harvard Apparatus, Holliston, MA) and a heat lamp. The composition of control perfusate (n=4 mice) is ((in mM) NaCl 124, KCl 4.7, CaCl₂·2H₂O 2.5, MgSO₄·7H₂O 1.2, KH₂PO₄ 1.2, NaHCO₃19)+Inulin (12.76 mM; 3.3 mg/mL)) while that of the LGG perfusate (n=4 mice) is KRB+LGG (3.3×10⁸ CFU/mL)+Inulin (12.76 mM; 3.3 mg/mL). After 4 hr perfusion, the ileum was immediately harvested for RNA sequencing.

DSS induced mild colitis treatment with LGG metabolites. To assess in vivo impact of metabolites on gut barrier function, conventional C57BL/6 mice (8 weeks of age) were administered with 2.5% dextran sodium sulfate (DSS) in drinking water to induce mild colitis. Animals were intraperitoneal injected with either PBS vehicle, 30 mg/kg methylnicotinamide chloride, or 3.6 mg/kg argininosuccinic acid dissolved in sterile PBS at Day-1 and Day 4 of the experiment. Body weight was recorded daily. At Day 7, 4 hours before sacrifice and collection of blood, animals were gavaged with 600 mg/kg with 4 kDa FITC-dextran. Blood was collected in 5% EDTA and subsequently centrifuged at 3000 g for 5 minutes for plasma collection. FITC readings were collected by diluting 25 μl of plasma in 75 μl of PBS and analyzed using a Glomax (Promega, GMMTE9032) with Ex490/Em510-570 filter cube module. Serum concentration of interleukin-6 (Cat. No. Biolegend 431304) was also determined. Ileum and colon sections were collected, fixed in 4% paraformaldehyde, processed for embedding in paraffin wax. Pathohistology of intestine was observed by routine H&E staining.

### Probiotic Lgg Administration

Lactobacillus rhamnosus GG (LGG) (ATCC 53103) was purchased from American Tissue Culture Collection (ATCC, Manassas, Virginia, USA). LGG was cultured at 37° C. in MRS broth (Fisher Scientific, Pittsburgh, PA, USA) following the manufacturer's protocol. The cultured broth was then centrifuged at 1200 g for 10 min to pellet the LGG. For LGG colonization, one cage of GF trp− mice and one cage of GF trp+ were orally gavaged with 200 μl of LGG (10⁸ CFU/ml) in sterile PBS (LGG trp− and LGG trp+, respectively) while control GF received 200 μl sterile PBS (PBS trp− and PBS trp+).

### Fecal DNA Extraction

Fecal DNA was extracted following the manufacturer's instruction with the Purelink Microbiome DNA Purification Kit (Invitrogen, Carlsbad, CA, USA). Samples were homogenized in lysis buffer using a bead homogenizer (Thermo Fisher, Waltham, MA) and centrifuged at 14000 g for 2 min. and the supernatants collected in a 1.5 mL microcentrifuge tube then mixed thoroughly with binding buffer. Then, samples were transferred onto a spin column for purification and eluted in 100 μl elution buffer (Invitrogen by Thermo Fisher). DNA concentrations were determined by spectrophotometry (Isogen ND-1000 Nanodrop, Wilmington, DL) for later analysis to determine bacterial concentrations by qPCR.

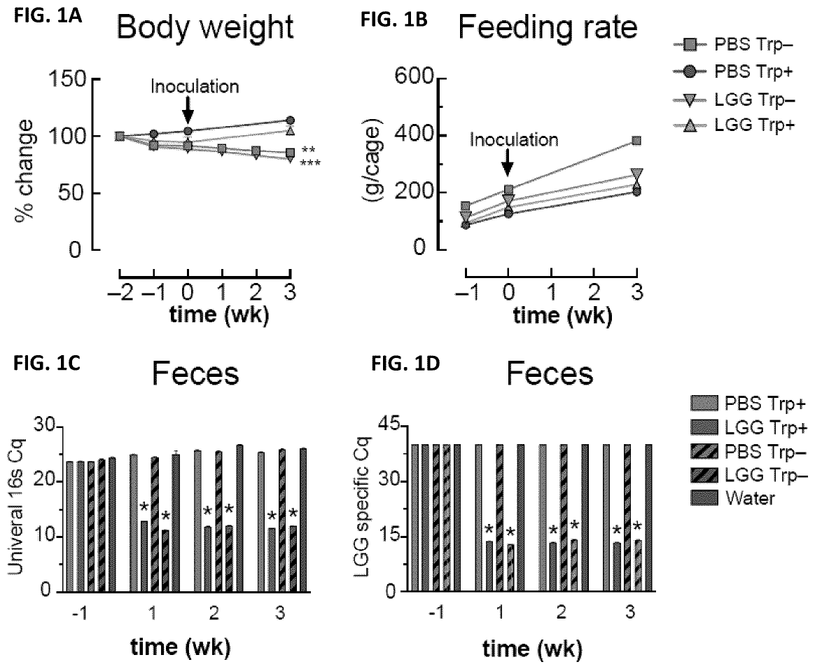

We monitored gnotobiotic status over 4 weeks, once weekly, by bacterial transcript analysis of the fecal matter. No significant bacterial amplicon (using universal primers to amplify the bacterial 16S rRNA gene) was detected in pre-inoculation fecal samples (FIG. 1C, left panel), indicating that all mice were germ-free before inoculation. Using LGG specific primers, we also showed that no LGG was present before inoculation (FIG. 1C, right panel). After inoculation, the average Cq of fecal 16S rRNA and of LGG sequence in the feces of PBS trp+ and PBS trp− were similar to that of the negative control (water) throughout the experimental period (1-wk. 2-wk and 3-wk post-inoculation (PO)). In contrast, a much lower Cq was found in LGG-inoculated mice, regardless of diet (LGG trp+ and LGG trp−) throughout the post-inoculation period. Here, the average Cq of fecal 16S rRNA and of LGG-specific primers were around 10-12. We also monitored LGG colonization status in individual mice (FIG. 1D), and observed that every mouse had relatively comparable Cq's when using 16S (left panel) or LGG-specific (right panel) primers, implying that they had relatively equivalent luminal LGG.

### Quantitative LGG Monocolonization and Validation of Germ-Free by QPCR

LGG was quantified in fecal samples by qPCR (Agilent AriaMx Real-time PCR system), using the following LGG-specific primers: forward, 5′-CGCCCTTAACAGCAGTCTTC-3′ (SEQ ID NO: 1) and reverse, 5′-GCCCTCCGTATGCTTAAACC-3′ (SEQ ID NO: 2). The 16S rRNA quantitative PCR used to quantify non-specific bacteria (compared to negative control distilled water) had the following universal primers: 16S rRNA forward 5′-AGAGTTTGATCCTGGCTCAG-3′ (SEQ ID NO: 3), and reverse, 5′-CTTGTGCGGGCCCCCGTCAATTC-3′ (SEQ ID NO: 4). The qPCR reaction was performed using SYBR Green (Thermo Fisher) as follows: 95° C. for 5 min, followed by 45 cycles at 95° C. for 10 sec, 65° C. for 15 sec, and 72° C. for 15 sec, and a final extension at 95° C. for 5 min, 65° C. for 1 min, and 98° C. for 30 sec.

### RNA Isolation and Sequencing

Total RNA was isolated from mouse ileum then purified with RNeasy Plus universal kit (Qiagen, Hilden, Germany) following manufacturer's instructions. The extracted RNA, assessed by using a bioanalyzer nanochip (Agilent Technologies, Santa Clara, CA), was of high quality, with an average±SE RIN of 8.6±0.56. For global assessment of mouse RNA levels, total RNA was subjected to two rounds of poly(A) selection using oligo (dT) 25 magnetic beads (New England Biolabs [NEB], Ipswich, MA). Illumina-compatible RNA-seq libraries were constructed using the NEBNext Ultra II RNA library prep with sample purification beads (Cat. No. E7775) and NEB Next Multiplex Oligos for Illumina (dual index primers set 1; Cat. No. E7600) according to the manufacturer's instructions. Poly(A) selection and library quality were assessed using TapeStation 2200 (Agilent Technologies, Santa Clara, CA), and libraries were quantified using Qubit 4.0 fluorometer (Thermo Fisher, Waltham, MA). The prepared libraries were sequenced on an Illumina NextSeq 500 instrument (Illumina, San Diego, CA). CLC Genomics Workbench version 20.0.4 (Qiagen, Venlo, Netherlands) was used for RNA-seq analysis.

Extraction of Polar Metabolites from Feces, Liver and Serum

Frozen fecal matter and liver were weighed and then each sample was deposited into a test tube containing extraction buffer (acetonitrile:methanol:water (40:40:20) in 0.5% formic acid) at a ratio of 75 ml buffer per mg of sample. Samples were then sonicated (4° C., 10 sec) and centrifuged at 17000 g, for 2 min. After centrifugation, the supernatant was diluted four-fold with extraction buffer then neutralized with 15% NH₄HCO₃ prior to LC-MS analysis.

As for serum, polar metabolites were initially extracted by incubating samples with methanol (1:4 ratio) in −20° C. for 20 min. After centrifugation (17000 g. 2 min), the supernatant was transferred to new tube as first extraction, while the pellet was retained in the tube and later being extracted with extraction buffer for 10 min on ice (acetonitrile:methanol:water (400:400:200)). Thereafter, this solution mixture was centrifuged (17000 g. 10 min), and the supernatant, then was combined with the first extraction. Extracts were stored at −80° C. for a few day and later analysis by LC-MS.

### LC-MS Metabolomics Analysis and Metabolites Identification

The LC-MS analysis was performed on hydrophilic interaction chromatography coupled with electrospray ionization to the Q Exactive PLUS hybrid quadrupole-orbitrap mass spectrometer (Thermo Scientific), as previously described (Kim et al 2021). Thereafter, the targeted metabolite data analysis was performed in MAVEN (Melamud et al., 2010). The compound identification was based on the accurate mass and the retention time learned from in-house chemical collection. The untargeted analysis was performed in Compound Discoverer (Thermo Scientific). The metabolomics datasets were analyzed by MetaboAnalyst 5.0. Herein. PCA and VIP analyses were performed to visualize differences in patterns of metabolite levels among samples, and to identify metabolites important in separating any two sets of samples. VIP score cut-off is 1.2 (Kim et al 2021).

### Intestinal Crypt Isolation and Organoid Culture

Intestinal organoids were isolated as previously described (Das et al., 2015; Pearce et al., 2018). Briefly, crypts were isolated from small intestine of conventional house C57BL/6 male mice (Taconic Laboratories, Hudson, NY) by shaking in isolation buffer (2 mM EDTA in PBS), then, crypts were harvested, processed, and suspended in crypt culture medium (CCM), which consisted of advanced DMEM/F12 (Life Technologies, Carlsbad, CA) supplemented with serum-free B27 (1:50; Life Technologies), N2 (1:100; Life Technologies), N-acetylcysteine (1 mM; Sigma-Aldrich, St. Louis, MO), recombinant murine epithelial growth factor (EGF, 50 ng/ml; Peprotech, Rocky Hill, NJ), Noggin (100 ng/ml; Peprotech), R-Spondin (500 ng/ml; Peprotech), Glutamax-I supplement (1:100; Life Technologies), penicillin/streptomycin (500 μg/ml; Life Technologies), and HEPES (10 μM; Life Technologies). Crypts were resuspended in 70% Matrigel (density ˜450 crypts per dome, 2 domes per well in 24-well plate) and allowed to solidify at 37° C. for 20 min. CCM (1.5 mL) was then added to each well. Cultures were incubated in a humidified incubator at 37° C., and CCM was changed daily. Intestinal organoid treatment with LPS and metabolites

We had 2 series of metabolite-treated organoid experiments, i.e., 1) organoid from conventional C57BL6 mice and 2) Ocln KO and littermate WT control as a gift from Prof. Jerrold Turner (Harvard University). LPS and all metabolites were purchased from Sigma Aldrich (St. Louis, MO, USA): LPS Cat. No. L2630, indole-3-propionic acid (IPA) Cat. No. 57400-5G-F, indole-3-acetamide (IAM) Cat. No. 286281, 1-methynicotinamide (MNA) Cat. No. SML0704, carnosine (CARN) Cat. No. C9625, and L-argininosuccinic acid (ASA) Cat. No. A5707. Seeding is designated as day 0. On day 3 of culture, the existing media in the wells were completely removed before adding treatments. LPS (1 μg/mL) with or without metabolites, i.e., IPA (0.1 mM) (Li et al., 2021), IAM (200 μM) (Vyhlidalova et al., 2020), MNA (12 ng/ml) (Liu et al., 2015), CARN (20 mM) (Boldyrev et al., 2013) or ASA (1 μM) (Erez et al., 2011) were each added in CCM at known physiological concentrations, incubated for 48 hours, thereafter, organoid was harvested for RNA extraction, and paraformaldehyde fixation for immunostaining.

RNA Extraction from Organoid and RT-QPCR Analysis

Total RNA was extracted from the intestinal organoids using a commercially available kit (RNeasy Microkit; Qiagen, MD, Cat. #74004) following manufacturer's instructions. mRNA samples were converted to cDNA using Bio-Rad's iScript Reverse Transcription Supermix for RT-qPCR (Bio-Rad Laboratories, Hercules, CA, Cat. #1708840). cDNA was then used for RT-qPCR (Mx3000P. Stratagene, La Jolla, CA) to analyze several genes using Maxima SYBR Green qPCR Master Mix (Thermo Fisher Scientific, Waltham, MA) and primers (Integrated DNA Technologies, Coralville, IA). Thermal profile for three-step cycling was 10 min at 95° C. for initial denaturation; then 40 cycles of 15 sec at 95° C. for denaturation, 30 sec at 65° C. for annealing, and 30 sec at 75° C. for extension. Primers used are as follows: β-actin (Actb), forward 5′-TTGTTACCAACTGGGACGACATGG-3′ (SEQ ID NO: 5), reverse 5′-CTGGGGTGTTGAAGGTCTCAAACA-3′ (SEQ ID NO: 6); ZO-1 (Tjp1), forward 5′-GGGAGGGTCAAATGAAGACA-3′ (SEQ ID NO: 7), reverse 5′-GGCATTCCTGCTGGTTACAT-3′ (SEQ ID NO: 8), Ocln, forward 5′-ATTCCATCAGTTTCCTATCT-3′ (SEQ ID NO: 9), reverse 5′-ACCAGGACCTTTCTTGAC-3′ (SEQ ID NO: 10); Lyz1, forward 5′-ATG GCT ACC GTG GTG TCA AG-3′ (SEQ ID NO: 11), reverse 5′-CGG TCT CCA CGG TTG TAG TT-3′ (SEQ ID NO: 12); Tff3, forward 5′-TAA TGC TGT TGG TGG TCC TG-3′ (SEQ ID NO: 13), reverse 5′-CAG CCA CGG TTG TTA CAC TG-3′ (SEQ ID NO: 14). All samples were normalized to β actin expression. Gene expression levels are depicted as fold change relative to the control group.

### Permeability Assay

Permeability assays followed earlier work (Pearce et al., 2018). Briefly, all media were removed and then organoid were incubated with CCM containing 4 kDa FITC-dextran (Sigma-Aldrich, St. Louis, MO, Cat. #46944) at a final concentration of 1.25 UM for 30 minutes at 37° C. and at 5% CO₂. After the 30-minute incubation, the organoids were quickly washed (˜ 10 times) with PBS. A fluorescent microscope (Nikon Eclipse TI, NY) at a 10× magnification was used to observe and capture the fluorescence within the lumen of the organoids. Using the same area as the lumen, three different background samples are measured randomly around the lumen. The three background samples measured are averaged and then subtracted from the measured fluorescence of the lumen. Both a brightfield and a FITC image were captured (6 fields/dome, 4 domes/treatment). The quantification of the fluorescence was conducted using the ImageJ 1.53 (NIH, Bethesda, MD).

### Immunohistochemistry

Immunocytochemistry followed earlier work (Pearce et al., 2018). Briefly, organoids were extracted from Matrigel by using cell recovery solution (Corning, Cat. No. 354253), processed then fixed in 4% paraformaldehyde for 15 min and dehydrated with 70% ethanol and kept in 4° C. until embedded in paraffin. On the day of embedding, organoids were gently spun down to a pellet (250 g for 5 min), then the supernatant was removed, the pellet resuspended in agarose and embedded in paraffin. As for ileum segments, after dissection, luminal content was flushed out with PBS and the segment fixed in 4% paraformaldehyde for 24 h, washed with PBS, dehydrated and embedded in paraffin. Tissue-embedded paraffin was cut into 5-μm sections and put on superfrost plus slide, and air dried at room temperature overnight. Tissue sections were deparaffinized, gradually rehydrated with decreasing ethanol concentrations (10 min each), then rinsed with PBS for 5 min. Sections underwent antigen retrieval with sodium citrate buffer (20 min, pH 6.0, 95° C.), washed with PBS, then processed in blocking buffer containing 2% BSA, 0.1% Triton X-100, and 2% normal goat serum for 1 h (24° C.). Following blocking, sections were incubated with primary antibody (in blocking buffer overnight at 4° C., washed with PBS (3 times, 10 min each), followed by a 2-h incubation with fluorescence-conjugated secondary antibody then counterstaining with nucleic acid stain (DAPI), and mounted with ProLong Gold Antifade Mountant (ThermoFisher). Primary antibodies were rabbit monoclonal OCLN (Cat. No. AB216327, ThermoFisher), mouse monoclonal ZO-1 (Cat. No. 1A12, ThermoFisher), mouse monoclonal TFF3 (Cat. No. 14475882. ThermoFisher) and rabbit polyclonal to Lysozyme 1 (Cat. No. PU024-5UP, Biogenex). Secondary antibodies were goat anti-mouse IgG-Alexa fluor 488 (Cat. No. A11001, ThermoFisher), Goat anti-rabbit IgG-Alexa fluor 555 (Cat. No., AB150078), Goat anti-mouse IgG-Alexa fluor 555 (Cat. No., A21422). Alkaline phosphatase staining was done by commercial alkaline phosphatase substrate kit (Cat. No. SK-5100, Vector, ThermoFisher).

### MEtabolome-TRanscriptome Correlation Analysis (METRCA) CORRELATION ANALYSIS

The correlation analysis between metabolome (both negative and positive ionization mode of fecal matter, serum and liver samples from 3 weeks after inoculation) and ileum and liver transcriptomes was performed using the R program. Transcriptome data were filtered and only the differentially expressed genes with fold changes >1.5 and FDR-adjusted P values <0.05 were considered. In every comparison, pairwise Pearson correlation coefficients were calculated between genes and metabolites, and statistical significance of the correlation was obtained with the cor.test function in R. The P values were adjusted for multiple comparison using the FDR method. Metabolite-gene pairs with FDR-adjusted P values <0.05 were considered to be significantly associated. Heatmap was generated by the pheatmap package in R. Genes were clustered by hierarchical clustering. Magnitude of color represents number of associated genes.

### Statistical Analysis

Results are expressed as mean±SEM. Statistical analysis of differences between the groups and the interaction of LGG and trp effect were analyzed by two-way ANOVA and Tukey's post-hoc test. Statistical significance was accepted at the level of P<0.05.

The following examples are provided to illustrate certain embodiments of the invention. They are not intended to limit the invention in any way.

### Example 1: LGG Biomarkers

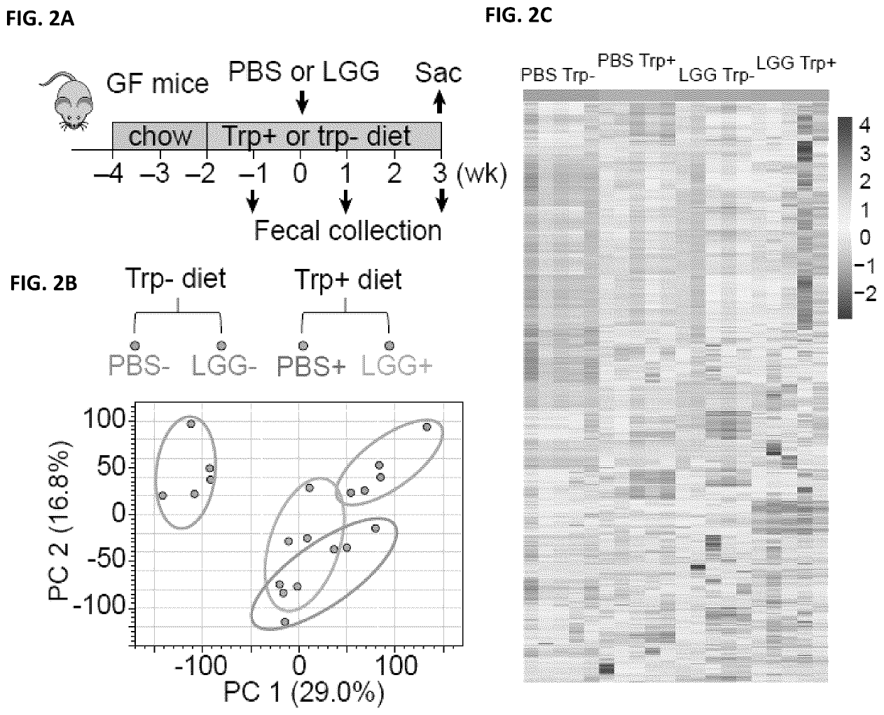

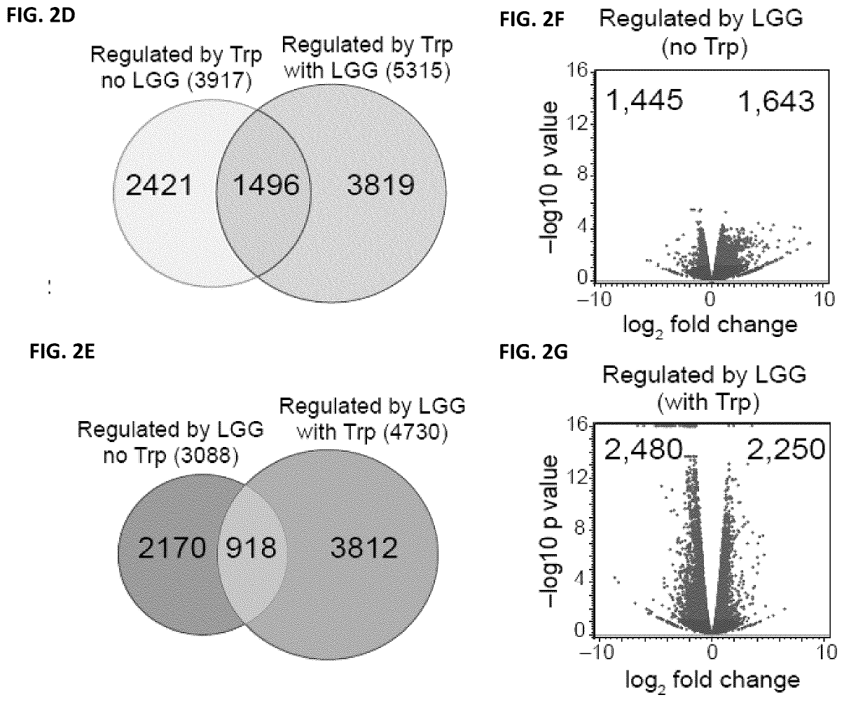

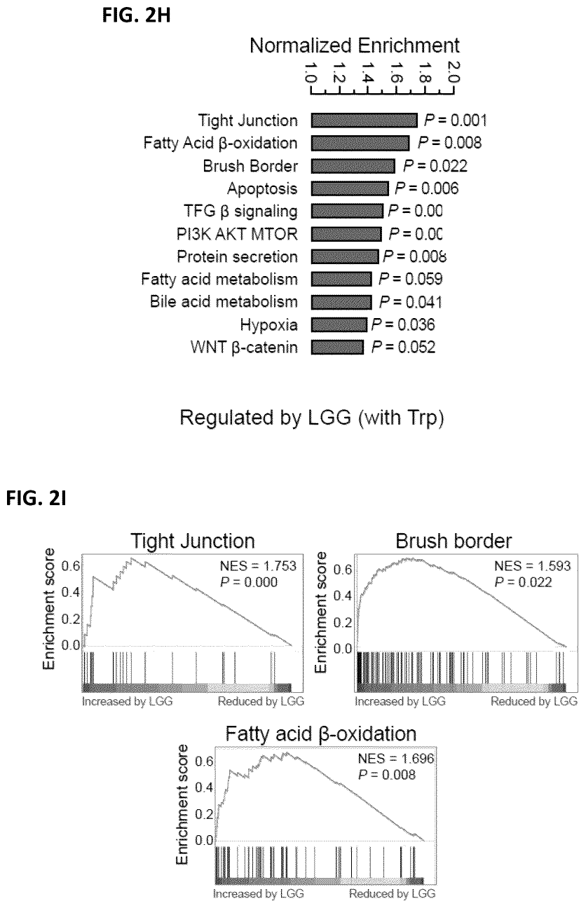

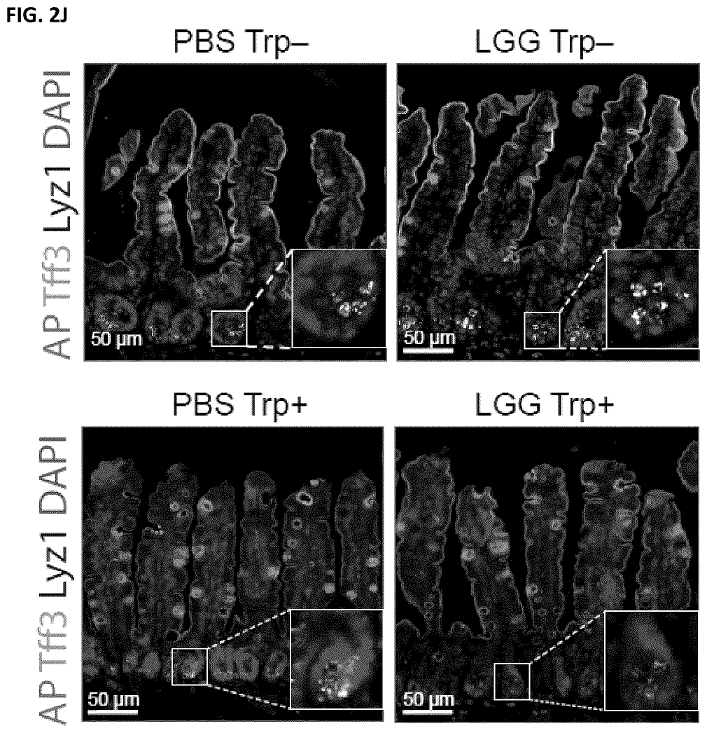

To investigate LGG-specific interactions with the host, we monocolonized germ-free (GF) mice with LGG. Two groups of GF mice were initially fed with either a trp (0.2%)-sufficient (trp+) or a trp− free (trp−) diet containing all the other matching ingredients. These two groups were divided into two cohorts: one monocolonized with LGG while the other administered with PBS to remain as GF controls (FIG. 2A). Over the course of experiment, mice fed with trp+ and trp− diet showed a modest weight gain or loss. Cumulative food consumption was slightly greater in trp− mice (FIG. 1A, 1B). Using quantitative (q)-PCR for universal 16S rRNA and LGG (Fontaine et al., 2015), we continuously monitored GF status of mice prior to inoculation and mice administered with PBS or LGG. LGG-inoculated mice were rapidly colonized by LGG with relatively equivalent abundance (FIG. 1C, 1D). Fresh fecal pellets were longitudinally collected for each mouse of all 4 groups during the 3 weeks of colonization. All mice were sacrificed on the same day when serum, liver and intestinal tissues were collected for metabolomic and transcriptomic analysis.

### Synergistic Impact of LGG and Dietary Tryptophan on Ileal Transcriptome

Bulk RNA-sequencing analysis was performed on ileal RNAs extracted from all 4 groups: PBS trp−; PBS trp+; LGG trp−; and LGG trp+ (n=5 mice per group). Principal component analysis (PCA) of gene expression levels showed that dietary trp altered ileal transcriptome in GF mice (compare PBS trp+ with PBS trp− (FIG. 2B)). LGG association, as expected, significantly altered ileal transcriptome under both trp+ and trp− conditions. A noticeable overlap can be seen between PBS trp+ and LGG trp−, indicating that dietary trp alone or LGG alone promoted similar ileal transcriptomic programs. Overall, the LGG trp+ mice were well-clustered and markedly separated away from the other 3 groups. Transcriptome heatmap demonstrated a marked alternation in LGG trp+ ileum (FIG. 2C).

The number of transcripts significantly affected by either LGG or dietary trp or by their combined effects are shown as Venn diagrams (FIG. 2D, 2E). Dietary trp affected 3917 ileal transcripts in GF mice, and 5315 ileal transcripts in LGG mono-associated mice. 1496 transcripts were affected solely by dietary trp with or without LGG (FIG. 2D). LGG colonization affected 3088 ileal transcripts in trp-free condition, and 4730 ileal transcripts in mice fed with trp-sufficient diets (FIG. 2E). 918 transcripts were affected by LGG, independently of dietary trp. LGG increases the effect of dietary trp, and trp increases the effect of LGG.

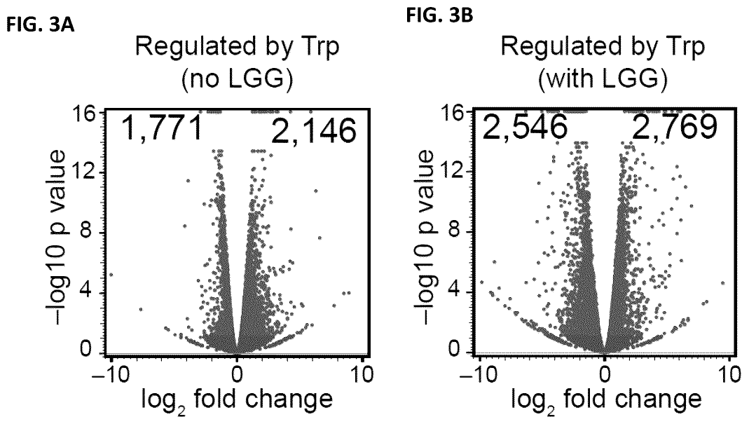

The synergy of LGG and dietary trp was reflected also by the magnitude of fold changes of gene expression. LGG altered positively or negatively 3088 ileal genes over 1.5-fold under dietary trp-free condition, while it altered 4730 genes with trp (FIG. 2F, 2G). Likewise, dietary trp changed 3917 genes over 1.5-fold in GF mice, while it changed 5315 genes LGG-associated mice (FIG. 3A, 3B). These results indicated that LGG robustly changes ileal transcriptome, and this effect was potentiated by dietary trp.

### LGG and Dietary Tryptophan Jointly Promote Enterocyte but Inhibit Paneth Cell Program

Gene Set Enrichment Analysis (GSEA) revealed that LGG and dietary trp together significantly increased gene programs related to enterocyte differentiation and function, such as TJ components, fatty acid oxidation and metabolism, brush-border transporters and enzymes, protein secretion, and bile acid metabolism (FIG. 2H, 2I). Although the villus epithelial morphology did not seem to differ among all 4 groups of mice, a noted reduction in Paneth cell number in LGG trp+ mice was observed when we stained for cell type specific markers (FIG. 2J).

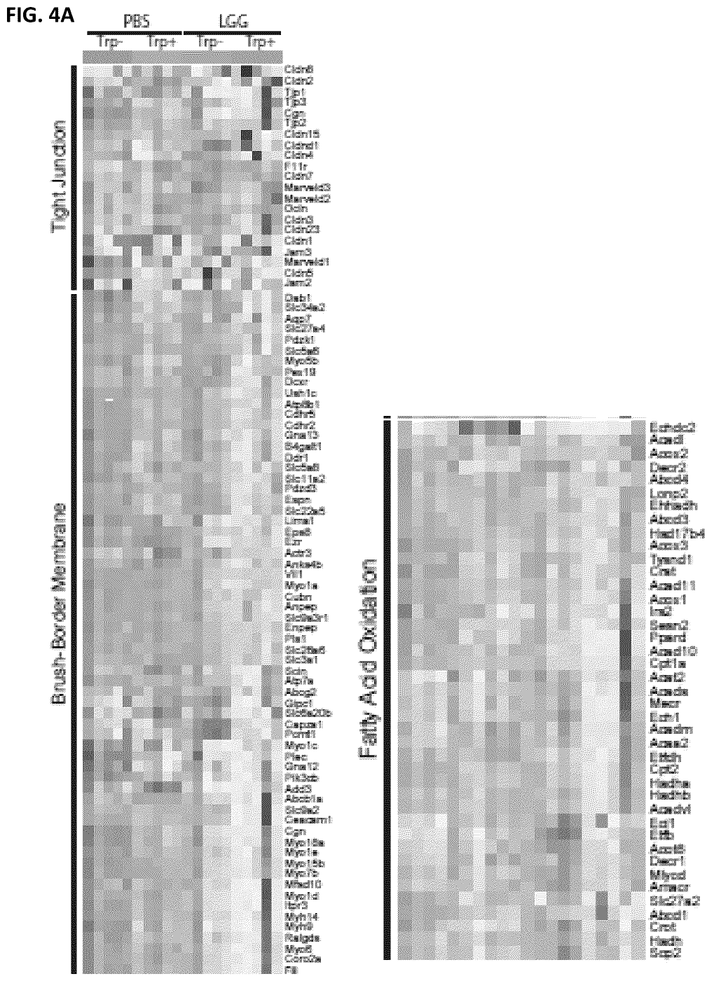

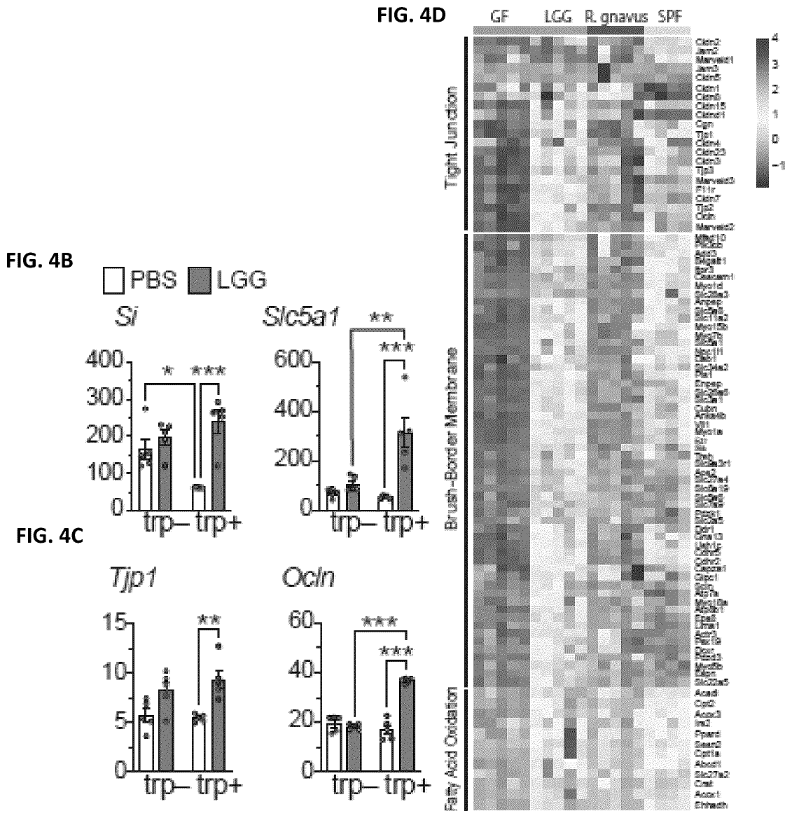

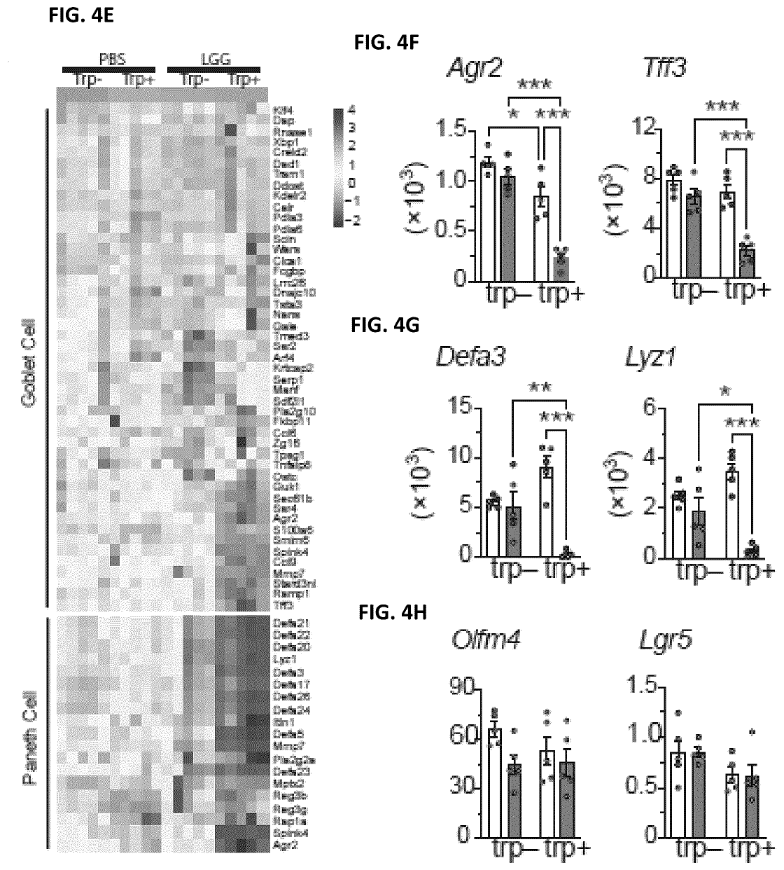

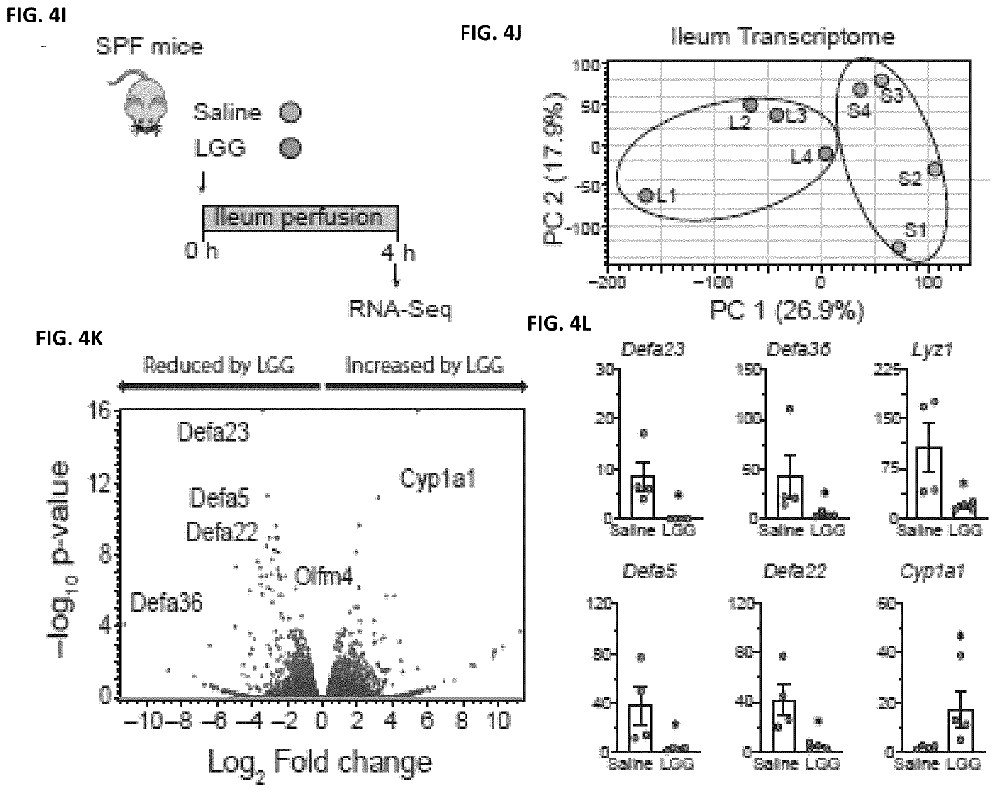

LGG promoted the abundances of most TJ genes in the presence of dietary trp (FIG. 4A, top panel). An overall similar synergistic effect was observed for major brush-border transporter and enzyme genes, as well as for fatty acid oxidation program (FIG. 4A, mid and bottom panels). The trp-effect in the presence of LGG (LGG trp+ compared with LGG trp−) promoted important brush-border proteins such as the water channel aquaporin Aqp8, carbonic anhydrase Car1, lactase Lct and phospholipase B1 (Plb1), whereas the LGG effect in the presence of trp (LGG trp+compared to PBS trp+) promoted strikingly the same genes Plb1 and Let, as well as other ones like deoxyribonuclease Dnase1 and fatty acid hydroxylase Fa2 h by >10-fold (Table 4A-4H listing top 10 genes altered in each comparison).

TABLE 4A
PSB Trp+ vs PBS Trp− (Top 10 up-regulation up in PBS Trp+)

| Entrez Gene Name | Symbol | Fold Change | FDR |
|---|---|---|---|
| nuclear receptor subfamily 0 | Nr0b2 | 95.7806 | 2.26E−08 |
| group B member 2 | | | |
| phospholipase A2 group IVC | Pla2g4c | 77.9981 | 1.79E−11 |
| | Pagr1b | 63.7846 | 1.37E−02 |
| fibroblast growth factor 15 | Fgf15 | 59.3814 | 0.00E+00 |
| ATPase H+/K+ transporting | Atp12a | 48.3530 | 1.30E−02 |
| non-gastric alpha2 subunit | | | |
| IZUMO1 receptor, JUNO | Izumo1r | 44.9740 | 2.038−02 |
| Major urinary protein 19 | Mup19 | 38.4611 | 7.61E−04 |
| sulfotransferase family 2A, | Sult2a2 | 37.4648 | 2.87E−02 |
| dehydroepiandrosterone | | | |
| (DHEA)-preferring, member 2 | | | |
| chymotrypsin like elastase 2A | Cela2a | 35.4347 | 5.53E−04 |
| H2A clustered histone 7 | H2ac7 | 28.1872 | 4.23E−02 |

TABLE 4B
PSB Trp+ vs PBS Trp− (Top 10 down-regulation down in PBS Trp+)

| Entrez Gene Name | Symbol | Fold Change | FDR |
|---|---|---|---|
| prostate stem cell antigen | Psca | −11.8627 | 291E−02 |
| D-box binding PAR bZIP | Dbp | −7.4379 | 0.00E+00 |
| transcription factor | | | |
| lipase family member N | Lipn | −7.2300 | 9.96E−04 |
| MAGUK p55 scaffold protein 4 | Mpp4 | −6.9881 | 3.93E−05 |
| t-complex-associated-testis | Tcte3 | −6.8303 | 1.52E−04 |
| expressed 3 | | | |
| exosome component 6 | Exosc6 | −6.7390 | 9.22E−04 |
| metallothionein 4 | Mt4 | −6.3675 | 4 04E−04 |
| arachidonate 12-lipoxygenase, | Alox12b | −6.0998 | 4.30E−02 |
| 12R type | | | |
| ripply transcriptional repressor 1 | Ripply1 | −6.0992 | 3.42E−02 |
| cation channel, sperm | Catsperz | −6.0989 | 4.36E−02 |
| associated 1 | | | |

TABLE 4C
LGG Trp+ vs LGG Trp− (Top 10 up-regulation up in LGG Trp+)

| Entrez Gene Name | Symbol | Fold Change | FDR |
|---|---|---|---|
| aquaporin 8 | Aqp [symbol] | 244.9915 | 0.00E+00 |
| carbonic anhydrase 1 | Car1 | 125.9166 | 1.95E−10 |
| UDP glucuronosyltransferase | Ugt2a3 | 90.7390 | 1.05E−11 |
| family 2 member A3 | | | |
| Mouse fertilized one-cell-embryo | C87414 | 81.4065 | 6.61E−03 |
| cDNA Mus musculus cDNA clone | | | |
| J0245E09 3′, mRNA sequence | | | |
| chymotrypsin like elastase 2A | Cela2a | 72.6154 | 7.85E−07 |
| lactase | Lct | 70.1878 | 0.00E+00 |
| mast cell protease 1 | Mcpt1 | 69.8168 | 1.64E−09 |
| pregnancy-specific glycoprotein | Psg27 | 69.5886 | 1.63E−02 |
| 27 | | | |
| ecionucleotide | Enpp7 | 68.6182 | 0.00E+00 |
| pyrophosphatase/phospho- | | | |
| diesterase 7 | | | |
| phospholipase B1 | Plb1 | 67.8200 | 0.00E+00 |

[symbol]

indicates data missing or illegible when filed

TABLE 4D
LGG Trp+ vs LGG Trp− (Top 10 down-regulation down in LGG Trp+)

| Entrez Gene Name | Symbol | Fold Change | FDR |
|---|---|---|---|
| kidney androgen regulated protein | Kap | −919.1554 | 2.20E−05 |
| myo-inositol oxygenase | Miox | −559.5971 | 9.99E−05 |
| acyl-CoA synthetase medium- | Acsm2 | −507.7349 | 1.26E−04 |
| chain family member 2 | | | |
| solute carrier family 12 member 1 | Sic12a1 | −496.8174 | 1.56E−04 |
| uromodulin | Umod | −447.0199 | 9.77E−07 |
| cadherin 16 | Cdh16 | −372.7976 | 2.98E−04 |
| secretoglobin, family 2B, member | Scgb2b15 | −351.1045 | 5.23E−05 |
| 16 | | | |
| solute carrier family 12 member | Sic12a3 | −196.6446 | 1.20E−03 |
| 3/Sodium-chloride symporter | | | |
| solute carrier organic anion | Sico1a6 | −141.5024 | 2.29E−03 |
| transporter family, member 1a6 | | | |
| solute carries family 22 member | Sic22a8 | −135.6330 | 2.82E−03 |
| 8/organic anion transporter 3 | | | |

TABLE 4E
LGG Trp− vs PBS Trp− (Top 10 up-regulation up in LGG Trp−)

| Entrez Gene Name | Symbol | Fold Change | FDR |
|---|---|---|---|
| Myo-Inositol Oxygenase | Miox | 449.5861 | 1.27E−03 |
| acyl-CoA synthetase medium- | Acsm2 | 407.9112 | 1.43E−03 |
| chain family member 2 | | | |
| uromodulin | Umod | 244.5565 | 9.71E−05 |
| Kidney androgen-regulated | Kap | 170.2371 | 7.98E−05 |
| protein | | | |
| Solute Carner Family 12 | Sic12a1 | 166.1964 | 5.94E−04 |
| Member 1/Kidney-Specific | | | |
| Na-K-Cl Symporter | | | |
| aquaporin 2 | Agp2 | 129.1922 | 7.63E−03 |
| Cadherin-16 | Cdh16 | 124.5983 | 9.44E−04 |
| IZUMO1 receptor, JUNO | Izumo1r | 107.2814 | 6.51E−03 |
| solute carrier family 7 | Slc7a12 | 78.4187 | 1.62E−02 |
| (cationic amino acid | | | |
| transporter, y [symbol] system), | | | |
| member 12 | | | |
| solute carrier family 22 | Slc22a6 | 69.3474 | 2.02E−02 |
| member 6/organic | | | |
| anion transporter 1 | | | |

[symbol]

indicates data missing or illegible when filed

TABLE 4F
LGG Trp− vs PBS Trp− (Top 10 down-regulation down in LGG Trp−)

| Entrez Gene Name | Symbol | Fold Change | FDR |
|---|---|---|---|
| cytochrome P450, family 3, | Cyp3a41b | −56.4945 | 2.69E−02 |
| subfamily a, polypeptide 41B | | | |
| Sin3A associated protein 25 | Sap25 | −46.1752 | 3.03E−02 |
| H3 Clustered Histone 2 | H3c2 | −10.3131 | 1.88E−02 |
| Exosome C nponent 6 | Exosc6 | −8.7319 | 6.87E−04 |
| lymphocyte antigen 6 complex, | Ly6g | −7.9699 | 6.48E−04 |
| locus [symbol] | | | |
| H4 clustered histone 2 | H4c2 | −7.9282 | 8.70E−03 |
| UDP glucuronosyltransferase | Ugt1a9 | −5.9795 | 1.45E−02 |
| family 1 member A9 | | | |
| H28 clustered histone 22 | Hist1h2bp | −4.1597 | 4.61E−02 |
| caboxypeptidase B2 | Cpb2 | −4.0219 | 8.57E−03 |
| PR/SET domain 14 | Prdm14 | −3.8330 | 2.97E−02 |

[symbol]

indicates data missing or illegible when filed

TABLE 4G
LGG Trp+ vs PBS Trp+ (Top 10 up-regulation up in LGG Trp+)

| Entrez Gene Name | Symbol | Fold Change | FDR |
|---|---|---|---|
| Pregnancy-specific | Psg27 | 28.0805 | 6.50E−03 |
| glycoprotein 27 | | | |
| UDP glucuronosyl- | Ugt2a3 | 25.0364 | 7.85E−07 |
| transferase family | | | |
| 2 member A3 | | | |
| GATA binding protein 4 | Gata4 | 23.5921 | 2.57E−08 |
| Phospholipase B1 | Plb1 | 19.0535 | 3.30E−08 |
| Lactase | Lct | 17.0726 | 1.07E−07 |
| solute carrier family 37 | Sic37a2 | 16.5035 | 2.04E−05 |
| member 2/Glucose-6- | | | |
| phosphate exchanger | | | |
| Deoxyribonuclease 1 | Dnase1 | 16.2996 | 1.28E−06 |
| tubulin tyrosine ligase like 9 | Ttll9 | 14.4576 | 2.61E−02 |
| cytochrome P450, family 2, | Cyp2b10 | 13.9760 | 1.98E−04 |
| subfamily b. polypeptide 10 | | | |
| Fatty acid 2-hydroxylase | Fa2h | 12.8942 | 2.77E−04 |

TABLE 4H
LGG Trp+ vs PBS Trp+ (Top 10 down-regulation down in LGG Trp+)

| Entrez Gene Name | Symbol | Fold Change | FDR |
|---|---|---|---|
| secretoglobin, family 28, | Scgb2b15 | −400.3428 | 4.32E−06 |
| member 15 | | | |
| Exosome Component 6 | Exosc6 | −114.0291 | 3.99E−03 |
| Delensin, alpha, 36 | Deta36 | −99.4407 | 0.00E+00 |
| Delensin, alpha, 41 | Deta41 | −66.5006 | 0.00E+00 |
| Resistin-like gamma | Retnig | −56.3996 | 1.11E−02 |
| cytochrome P450, family | Cyp3a41b | −51.4245 | 2.38E−02 |
| 3, subfamily a, | | | |
| polypeptide 41B | | | |
| heat shock protein 1 | Hspe1-rs1 | −47.4247 | 1.46E−02 |
| (chaperonin 10), | | | |
| related sequence 1 | | | |
| Major urinary protein 17 | Mup17 | −41.9176 | 1.88E−02 |
| germinal center associated | Gcat_2 | −37.8524 | 2.17E−03 |
| signaling and motility | | | |
| defensin, alpha, 33 | Defa33 | −32.7975 | 0.00E+00 |

Other enhanced enterocyte programs (not shown) include elevations of eight peroxin genes (Pex2, 6, 7, 11a, 11 g, 13, 16, 19) that encode for enterocyte peroxisomes where fatty acids are catabolized, and ATP-binding cassette family genes (Abcd1, 3, 4 and Abcal, 2, 3, 6) that transport fatty acids in these peroxisomes. Virtually all major components of intestinal triglyceride and lipid metabolism are increased: Agpat1, 2, Mgat1, 2, 3, 4, 4b, 5, Dgat2, Mogat2 as well as the apical cholesterol transporter Npc111, intracellular cholesterol transporter Npc1, and long chain fatty acid transporters (Slc27a1, 2, 4, 5), essential for fat absorption from the lumen. Apolipoproteins Apob, Apobec 1 and Apoa1, 2, 5 required for chylomicron formation and lipid export from the intestine as well as the rate-limiting mitochondria outer membrane Cpt1 and inner membrane Cpt2 transporters of fatty acyl CoA are also elevated in LGG trp+ mouse ileum (FIG. 4A). Clearly, intestinal lipid metabolism is markedly enhanced by LGG.

Of note, the elevation of majority of LGG-promoted enterocyte genes, such as the brush-border transporter Sglt1 (Slc5a1) and TJ component Ocln, appeared strictly reliant on the presence of dietary trp (FIG. 4B, 4C). To examine whether the effects on enterocyte program were specific to LGG, we compared the ileal transcriptome among GF mice monocolonized by LGG, GF mice monocolonized by Ruminococcus gnavus (a commensal gut bacterium) and mice in specific pathogen free (SPF) condition. All these groups of mice were fed with trp+ diets. We found that the induction of a large number of enterocyte TJ genes (Ocln, Marveld2, Marveld3, Tjp2, Cldn1, Cldn7), brush-border transporters and enzymes (Npc111, Treh, Slc2a2, Slc5a1, several aquaporin genes), and fatty acid oxidation genes were uniquely regulated by LGG but not by R. gnavus or the SPF microbiome (FIG. 4D).

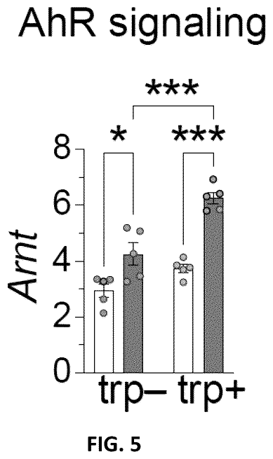

The elevated enterocyte program in LGG trp+ mice appeared to be at the cost of secretory cell programs, as a partial goblet cell gene set (Tff3, Ostc and Guk1, etc.) (FIG. 4E, 4F) and the entire Paneth cell gene set (Lyz1, Reg3b, Reg3 g, Pla2 g2a, Mmp7, and numerous defensin genes) (FIG. 4E, 4G) were reduced in LGG trp+ ileum. In contrast, LGG with dietary trp had no effect on Lgr5 and Olfm4, canonical transcripts of fast-cycling crypt based intestinal stem cells (ISCs) (FIG. 4H). Furthermore, the AhR nuclear translocator Arnt was also elevated in LGG trp+ ileum (FIG. 5).

To evaluate whether similar effects on IEC can be observed in conventional mice, we acutely perfused the ileum of conventionally housed mice with live LGG (3.3×10⁸ CFU/mL saline with 3.3 mg/mL inulin at 37° C.) for 4 hr (FIG. 4I). Control group was perfused with saline and inulin. Bulk RNA-Seq on LGG- or saline-perfused ileum showed separated transcriptomic profiles (FIG. 4J), with the most reduced transcripts being defensin family members (e.g., Defa 23, 36, 5, 22) in LGG perfused mice (FIG. 2K. L), indicating that live LGG can immediately suppress Paneth cell transcriptome. Cypla1, an AhR signaling target, was also significantly increased (FIG. 4L). Together, these data indicated that even though neither LGG nor dietary trp alone was able to alter IEC differentiation, LGG in conjunction with dietary trp promoted enterocyte program in vivo at the expense of secretory cell types.

### Identification of LGG-Dietary Tryptophan Regulated Fecal and Serum Metabolome

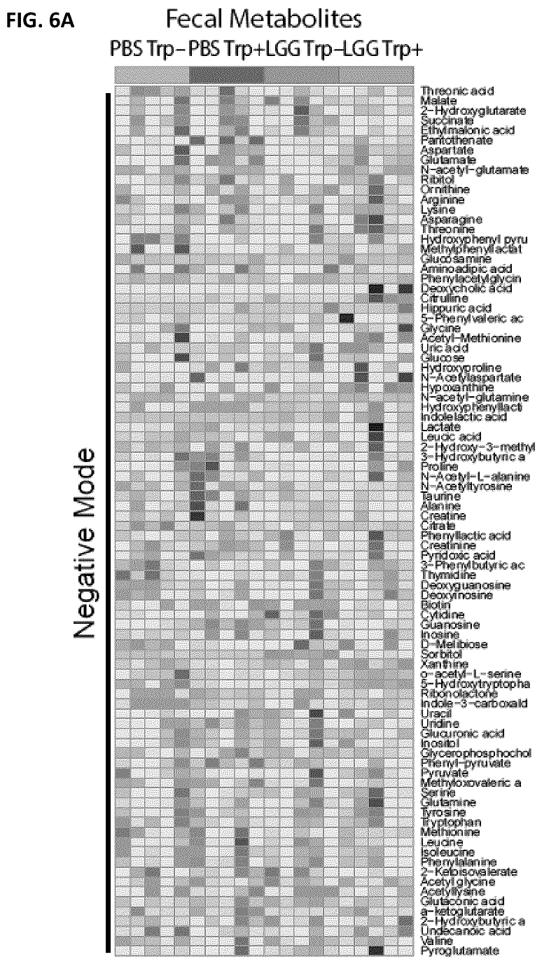

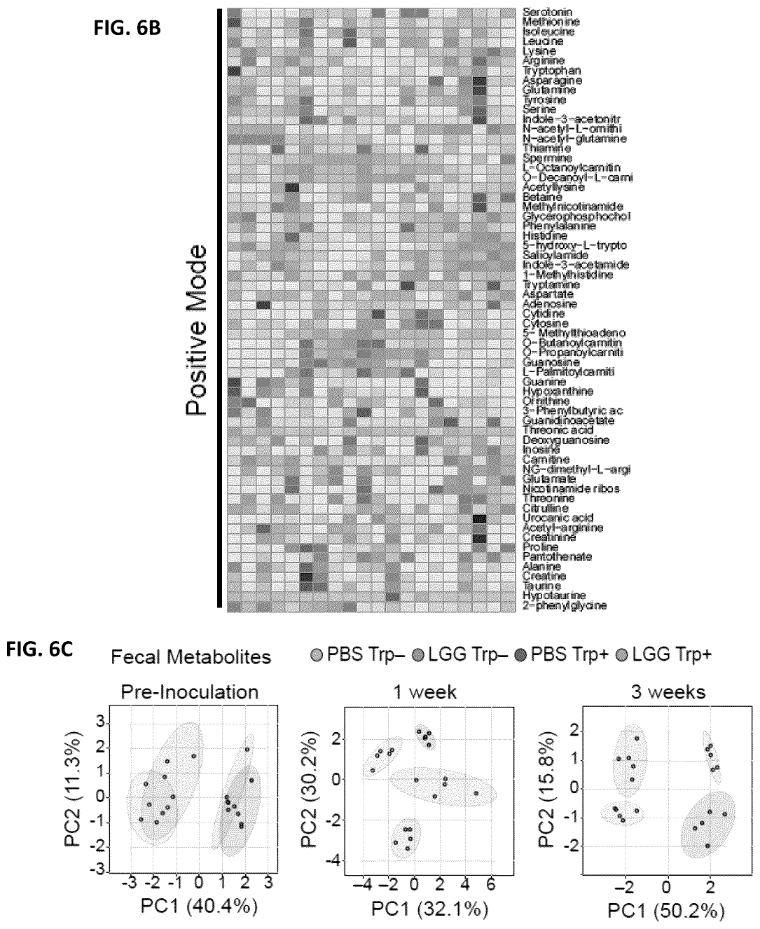

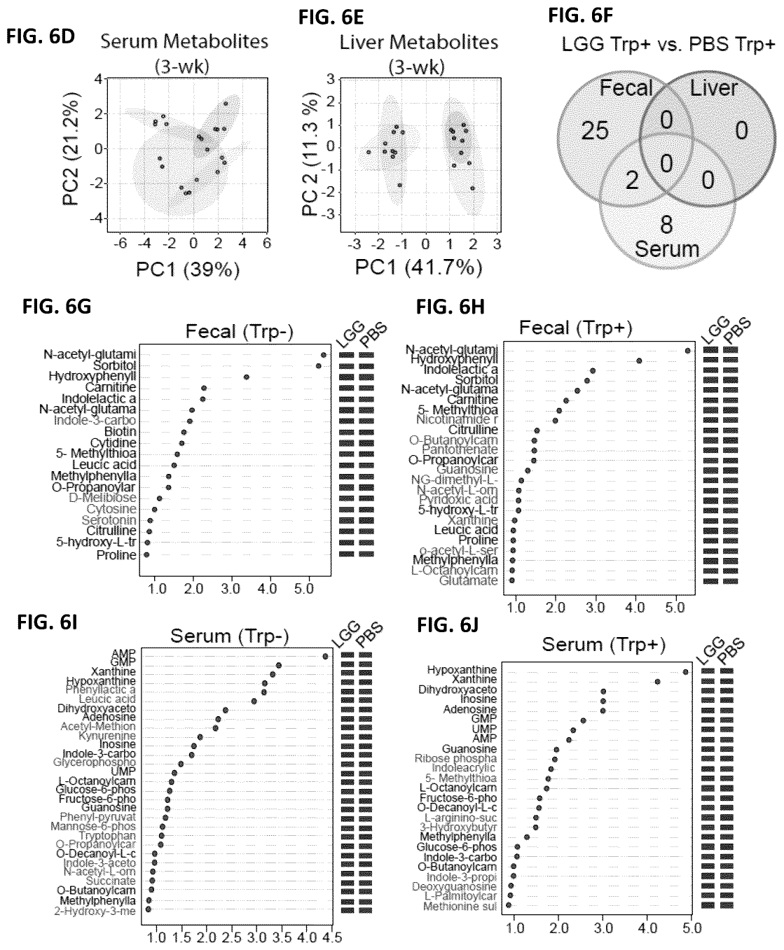

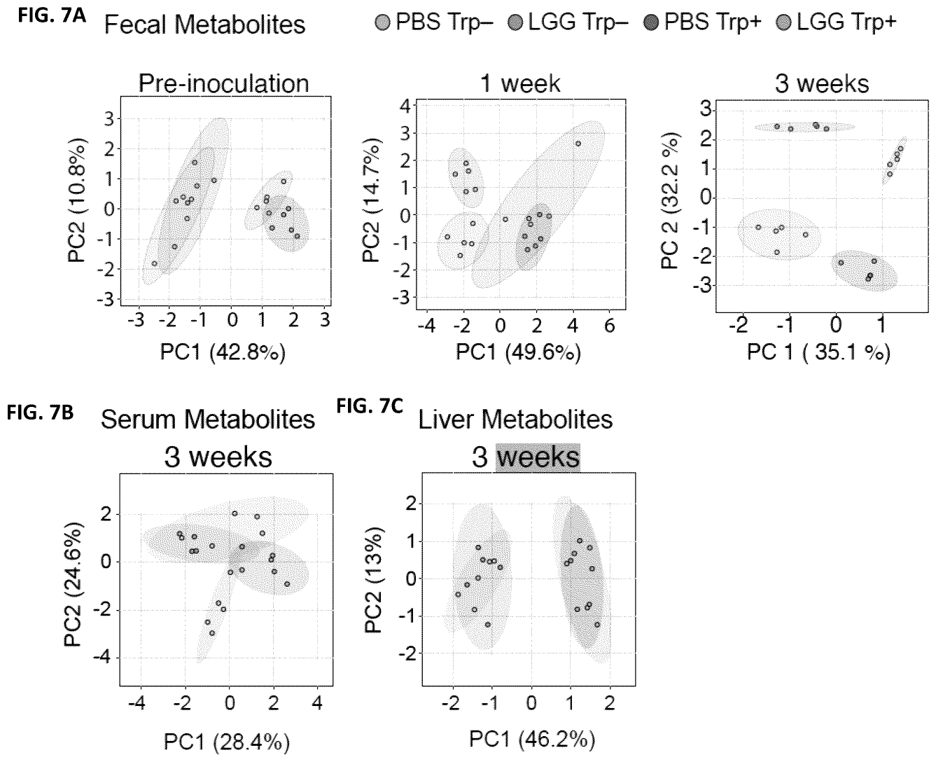

To identify metabolites that are derived from LGG-host-dietary trp interaction, we conducted an untargeted metabolomic analysis using liquid chromatography-mass spectrometry (LC-MS) that uncovered about 200 polar fecal metabolites under both negative and positive ionization modes that differed in abundance among the 4 groups (FIG. 6A-6B). Prior to LGG colonization, dietary trp, as expected, dictated the pattern exhibited by the fecal metabolome in GF mice (left panel, FIG. 6C). Within a week of LGG colonization, fecal metabolite profiles of the 4 groups shifted apart (middle panel, and the separations into four distinct groups persisted after 3 weeks (right panel). Although only metabolites in the negative mode are presented in these panels, similar changes were observed for metabolites in the positive mode (FIG. 7A). Thus, dietary trp and LGG robustly shape host intestinal luminal metabolome.

Serum and liver metabolome were analyzed when mice were sacrificed 3 weeks after colonization (FIG. 6D, 6E; FIG. 7B, 7C). Separation of serum metabolomic profiles was not as pronounced as that in the fecal metabolome, and there was noticeable overlap between LGG trp− and PBS trp− mouse serum metabolomes, indicating that LGG had modest impact on host serum metabolites without dietary trp. However, in the presence of dietary trp, LGG induced a marked alteration in serum metabolome. In contrast to fecal and serum metabolomes, liver metabolome was only affected by the status of dietary trp, with little contribution from LGG (FIG. 6E). When comparing the metabolites significantly changed in intestinal luminal, serum, and liver compartments, we noted that LGG in trp+ mice primarily affected fecal and serum metabolites, whose identities are largely different from each other (FIG. 6F). These data collectively indicate that LGG may metabolize dietary trp into derivatives that are differentially distributed in the luminal and serum compartments.

Variables Important in the Projection (VIP) analysis revealed the identities of driver metabolites for the inter-group differences in fecal, serum (FIG. 6G-6J) or liver (not shown) compartments. For fecal trp derived metabolites, LGG elevated indole lactic acid and 5-hydroxy-L-trp, independent of dietary trp status (FIG. 6G, 6H). Interestingly, LGG elevated indole-3-carboxaldehyde but reduced serotonin abundances only when dietary trp was deficient. Non-trp metabolites (red text) significantly increased (red rectangle) or decreased (blue text) by LGG under either trp− or trp+ condition, and those (black text) altered in both trp− and trp+ were uncovered (FIG. 6G, 6H). For serum trp derived metabolites, LGG reduced trp, kynurenine, and indole-3-acetonitrile in trp-mice, while increasing indoleacrylic acid and indole-3-propionic acid (IPA) in trp+ mice (FIG. 6I, 6J).

### LGG and Dietary Tryptophan Modify Selected Metabolites in Synergy

As the transcriptome analysis indicated a synergy in promoting the enterocyte program by LGG and dietary trp, we then searched for metabolites with similar pattern indicative of synergy. Dietary trp can be metabolized in host cells into kynurenine, 5-hydroxy-L-trp, and serotonin, etc, or converted by gut bacteria into various indoles and transported through blood stream into other tissues (FIG. 8A). We compared the abundance of all detectable trp derivatives in the intestinal lumen (fecal), serum and liver, among the 4 groups. While most trp derivatives were expectedly elevated in mice fed with trp+ diet, an LGG and trp combinatory effect could be observed for fecal 5-hydroxy-L-trp, and indole-3-acetamide (IAM) (blue text) (FIG. 8B), as well as for serum IPA and indoleacrylic acid (FIG. 8C). Interestingly, LGG increases fecal indole-3-lactic acid, a known trp derivative, in both trp− and trp+ conditions (FIG. 8B). In the liver, all trp metabolites were only affected by dietary trp status and not by LGG (FIG. 8D), reflecting the PCA (FIG. 6D).

Remarkably, synergistic effects were observed for certain fecal and serum non-trp metabolites whose abundances were robustly promoted by LGG in conjunction with dietary trp (FIG. 8E, 8F). Serum phenyllactic and leucic acid were promoted by LGG only when mice were fed with trp-deficient diet (FIG. 8G). Interestingly, while LGG-mediated suppression of fecal 5-methylthio-adenosine, sorbitol (FIG. 8H), and serum xanthine (FIG. 8J) were independent of dietary trp, LGG-mediated suppression of fecal guanosine, multiple carnitine-conjugated fatty-acid derivatives (red text) (FIG. 8I), serum L-arginino-succinate (ASA), 3-hydroxy-butyric acid, and ribose phosphate were dependent on dietary trp (FIG. 8K). These results demonstrated that LGG modifies selective host luminal and serum metabolites in the context of availability of dietary trp.

### Identification of Enterocyte Program-Correlating Metabolites

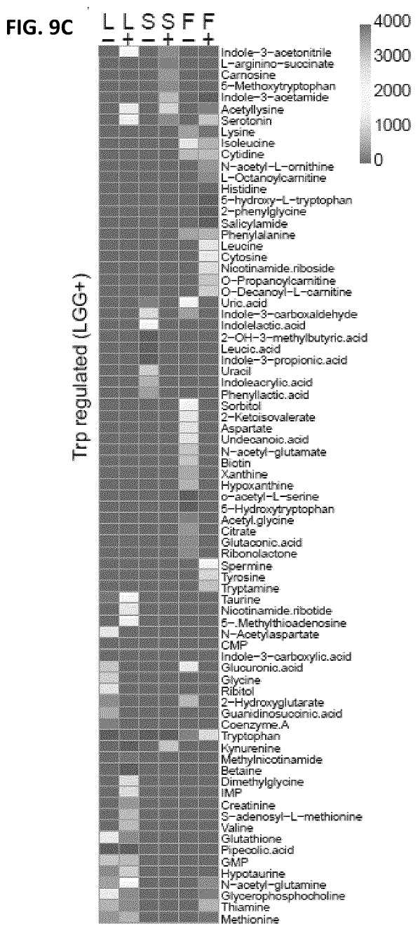

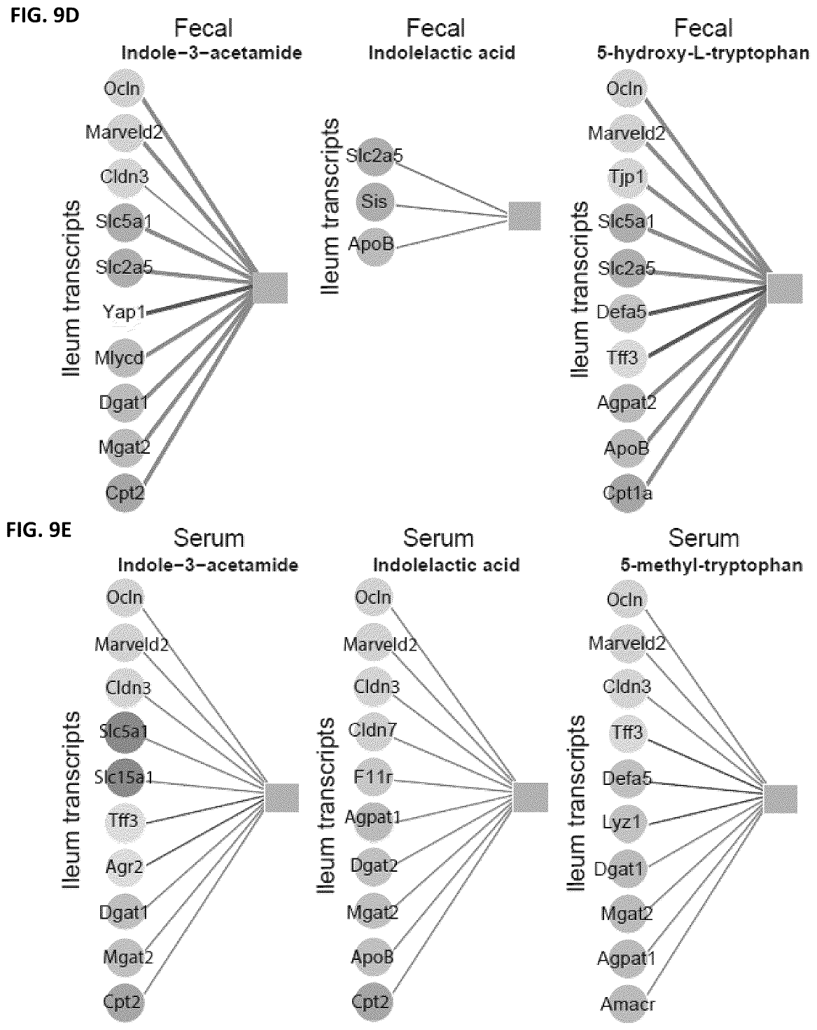

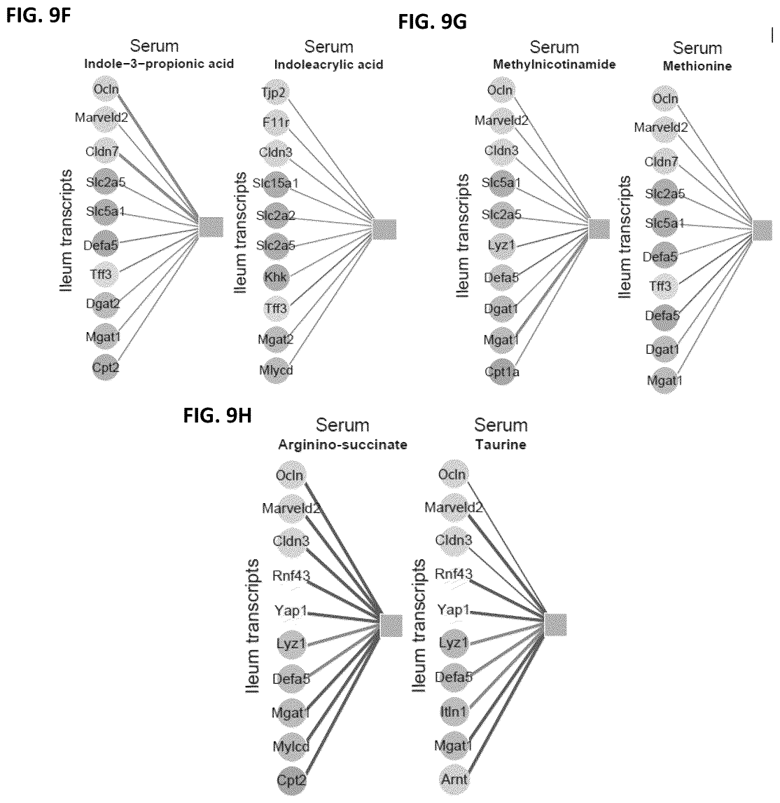

To uncover potential links between metabolite levels and observed changes in enterocyte program in LGG trp+ mice, we developed a R language-based MEtabolome-TRanscriptome Correlation Analysis (METRCA) bioinformatic pipeline. Based on the high-resolution metabolome and transcriptome results from each individual mouse, METRCA identified significant associations between levels of specific ileal transcripts with those of metabolites by comparing any two different experimental groups. In mice fed with trp-deficient diets (comparing LGG trp− and PBS trp−), METRCA identified limited numbers of LGG-regulated metabolites that correlate with transcriptomic changes (FIG. 9A). Heatmap scales represent the number of genes that a specific metabolite significantly correlates with. The correlation heatmap indicated that in absence of dietary trp, the changes in fecal D-Melibiose and sorbitol abundances were correlated with the most ileal transcripts, while serum glycerol-3-phosphate, glycerophosphocholine, and pyroglutamate were correlated with the most ileal transcripts (FIG. 9A). In mice fed with trp-sufficient diets, METRCA uncovered increased numbers of LGG-regulated metabolites (comparing LGG trp+ and PBS trp+) that significantly correlate with ileal transcripts (FIG. 9B). Similar metabolites, such as fecal sorbitol, also correlate with more ileal transcripts (please compare the heat intensity of sorbitol in FIG. 9A with 9B). Fecal 5-hydroxy-L-trp (identified in both positive and negative modes) and indole-3-acetamide (IAM) (positive mode only) were uncovered as prominent metabolites among trp derivatives that correlate with a large number of ileal transcripts. These ileal transcriptome-correlating metabolites were also identified when a METRCA comparison was made between LGG trp− and LGG trp+ mice, which interrogated dietary trp-regulated correlations in the presence of LGG (FIG. 9C). Numerous indoles (indole-3-acetonitrile, IAM, indole-3-carboxaldehyde, indolelactic acid, IPA, indoleacrylic acid) were found to be correlated with a large number of ileal transcripts (FIG. 9C).

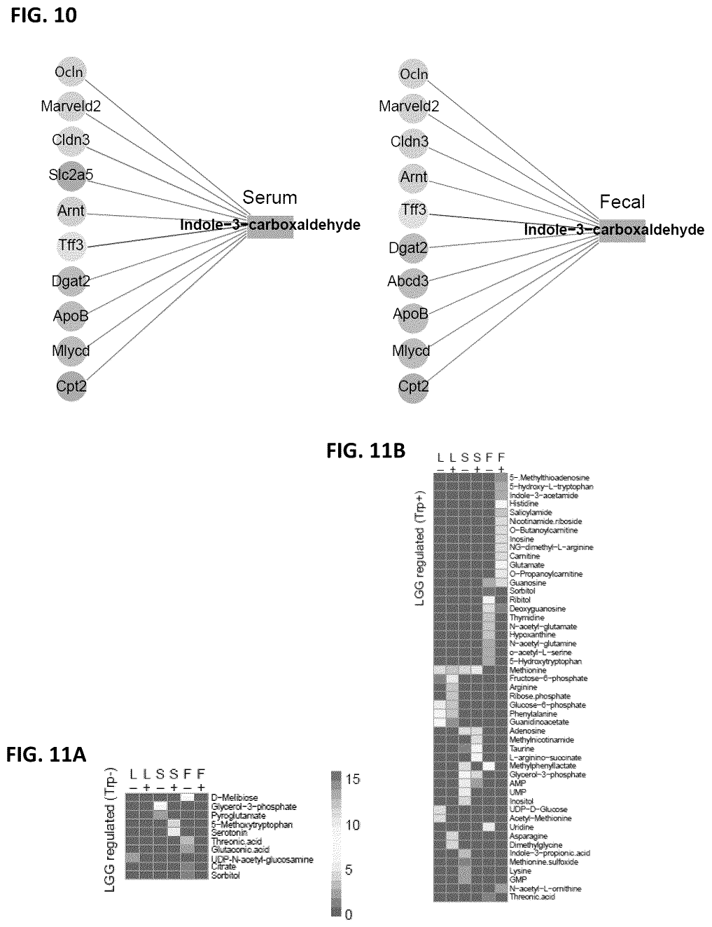

The majority of these correlative indoles belongs to the serum, although some, e.g., IAM, indole-3-carboxaldehyde and indolelactic acid, were found to correlate with ileal transcripts from both fecal and serum compartments. Some metabolites like fecal IAM and 5-hydroxytrp correlate with many transcripts in both LGG trp+ and LGG trp as well as LGG trp+ and PBS trp+ comparisons (thick lines). Interestingly, both fecal and serum IAM appeared to correlate with a largely similar set of transcripts for TJ, brush border, and lipid metabolism proteins, whereas indolelactic acid correlate with distinct ileal transcripts depending on their luminal or serum sources (FIG. 9D, 9E). Fecal and serum indole-3-carboxaldehyde also regulate similar TJ, lipid metabolism, Paneth cell and fat oxidation transcripts, but fecal is not correlated with most brush-border proteins (FIG. 10). These data indicate that effects mediated through certain serum metabolites may be different from fecal microbial metabolites. Fecal indolelactic acid correlates with fewer, mostly brush-border genes, while serum indolelactic acid correlates with a large number of TJ and lipid metabolism genes (FIG. 9D, 9E), indicating a polar component in some metabolite's impact on the mucosal epithelia. Serum 5-methyl-trp showed preferential positive correlations (blue line) with lipid metabolism gene network (FIG. 9E). Various trp and non-trp metabolites, while positively correlating with enterocyte gene network, exhibited negative correlations (red line) with goblet or Paneth cell specific genes (FIG. 9D-9G). Interestingly, we found several serum metabolites, such as ASA (identified in both comparisons FIGS. 9B and 9C) were negatively correlated with TJ and lipid metabolism genes but positively correlated with Paneth cell genes (FIG. 9H), indicating a negative impact on enterocyte differentiation.

### Identification of TJ-Correlating Metabolites

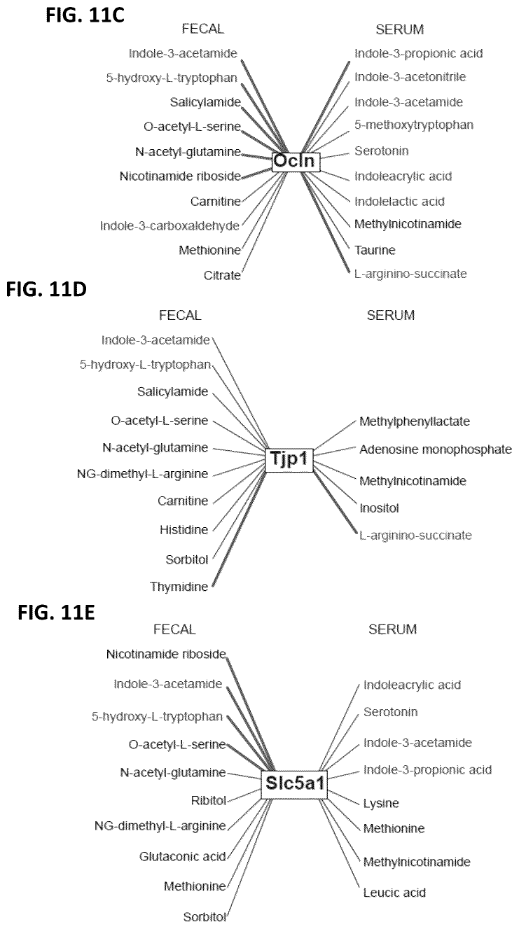

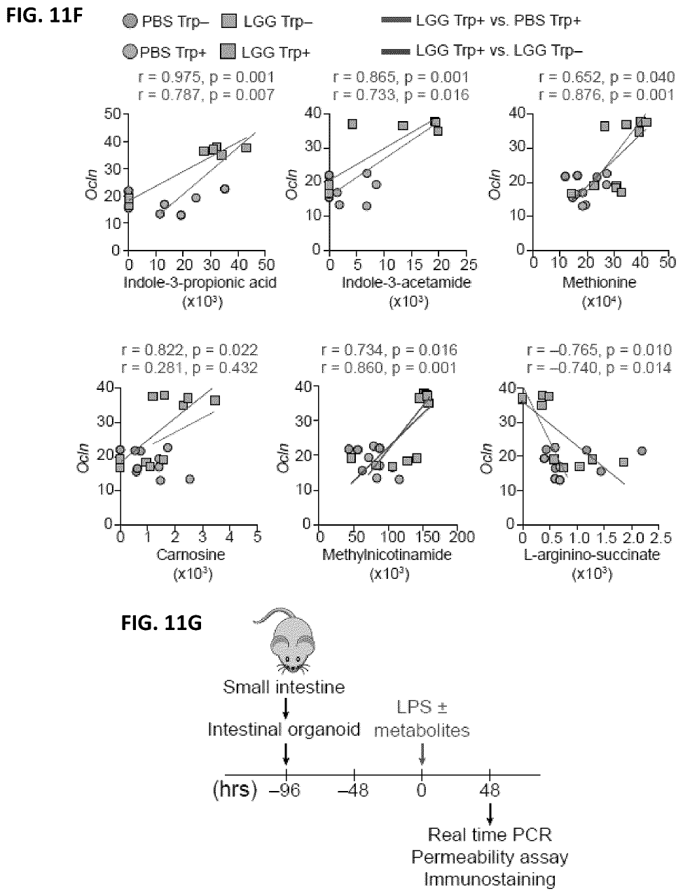

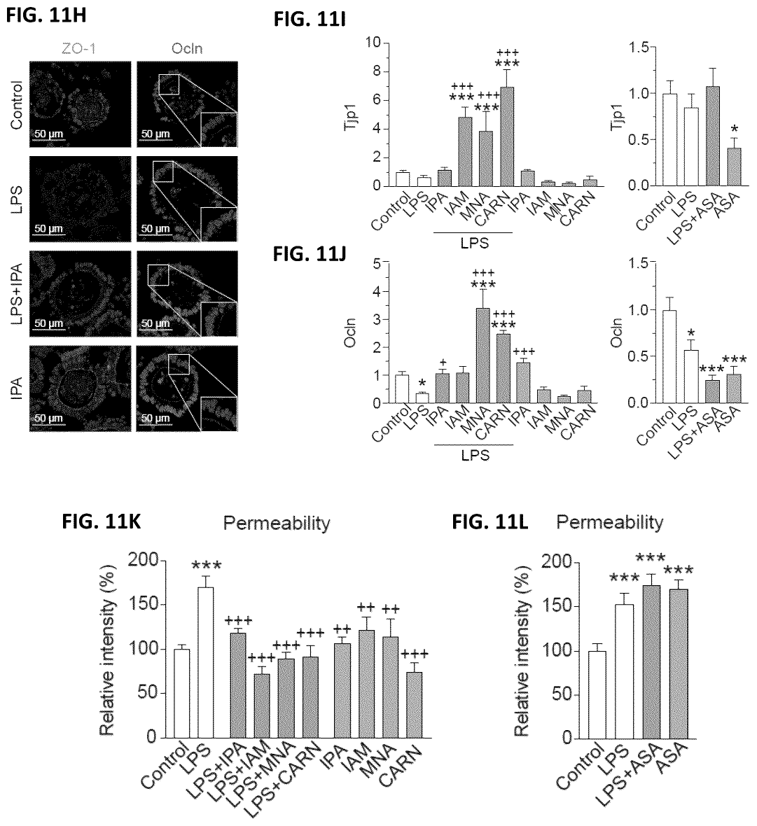

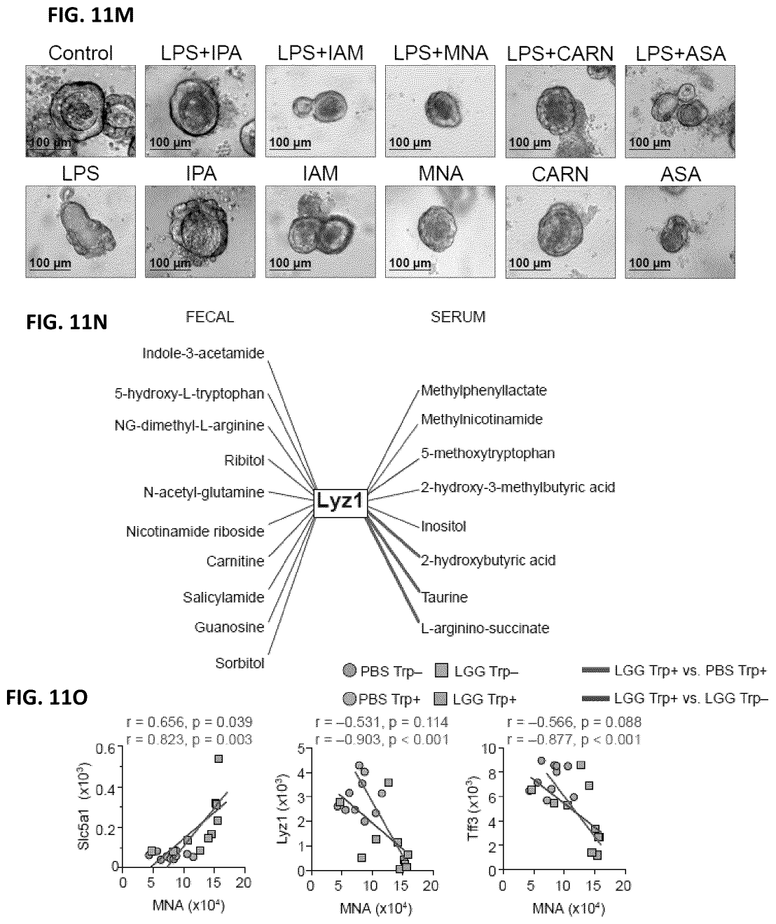

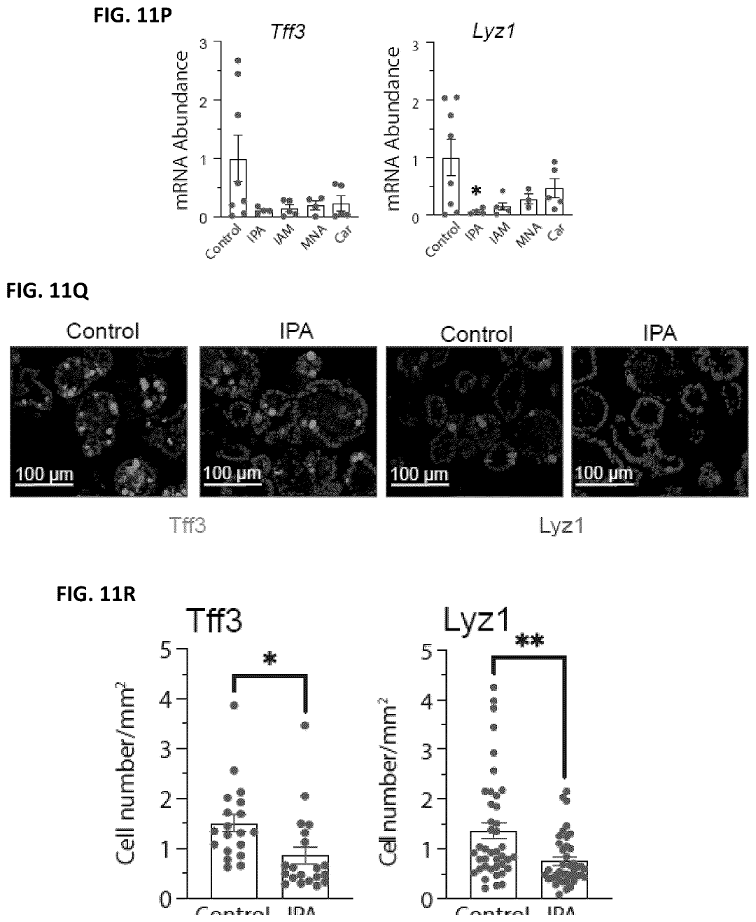

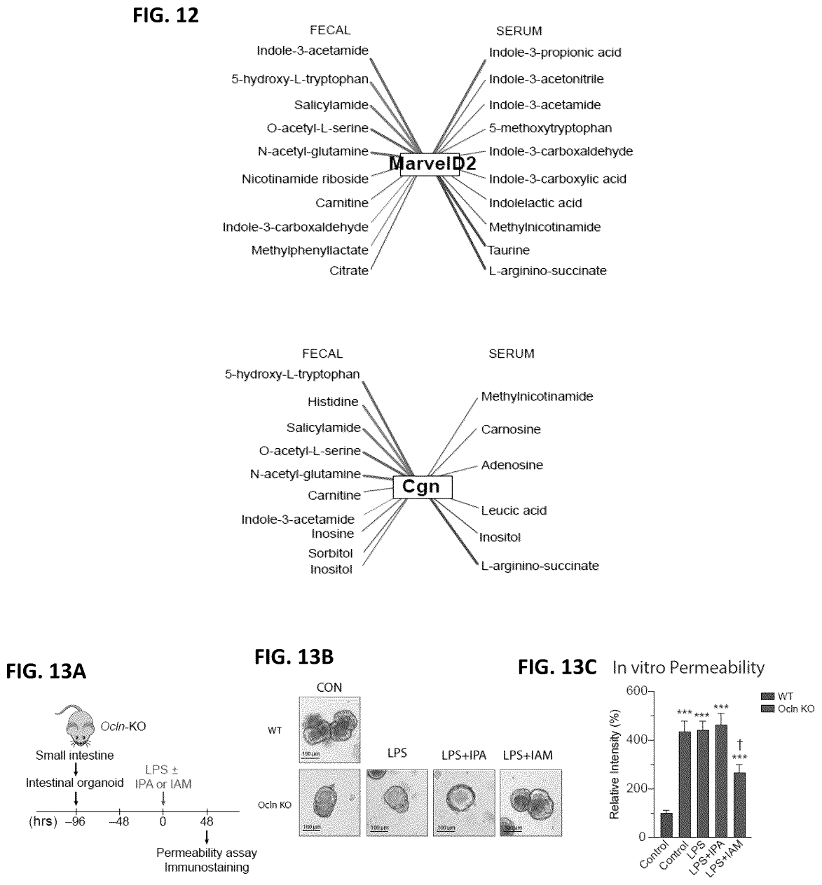

Because TJ gene expression was robustly increased in LGG trp+ mice, we next used METRCA to search specifically for metabolites that associate with a panel of 14 TJ genes that contribute to the paracellular leak pathway (Hollander and Kaunitz, 2020; Odenwald et al., 2017). In dietary trp-deficient condition, a small number of LGG-regulated metabolites were correlated with TJ genes, with serum 5-methoxytrp and serotonin being the major trp derivatives showing some correlations with a limited number of genes (FIG. 11A). In mice fed trp-sufficient diets, we uncovered 26 fecal metabolites and 15 serum metabolites (FIG. 11B), which significantly correlated with more TJ transcripts than the trp-deficient condition. For example, Ocln was positively correlated with a number of fecal and serum trp metabolites, with fecal IAM and serum IPA being the strongest ones (FIG. 11C). Similar correlative metabolites were found for other TJ genes, such as Tjp1, MarvelD2 (FIG. 11D, FIG. 12), and brush-border genes, such as Slc5a1 (FIG. 11E). Interestingly, serum ASA was found to be the strongest negative correlator with multiple TJ genes (FIG. 11C, 11D), but was not associated with brush-border genes (FIG. 11E, FIG. 9H). Representative correlation plots validated the significant positive or negative correlations of Ocln with various serum metabolites uncovered by METRCA (FIG. 11F).

### LGG Metabolites Regulate Intestinal Epithelial Permeability

We have developed a method assessing the TJ leak pathway using mouse enteroids (Pearce et al., 2018). We used LPS as an epithelial stressor to disrupt TJ and increase permeability. We test the TJ and barrier regulating effects of candidate metabolites by adding them into the culture (FIG. 11G) Remarkably, positive Ocln-correlators: IPA, IAM, methynicotinamide (MNA) and carnosine (CARN) increased TJ expression, in the presence of LPS, at the protein and mRNA levels, whereas negative correlator, ASA alone significantly reduced TJ expression (FIG. 11H-11J). We then found that the elevated or reduced TJ expression by these individual metabolites were reflected by increased or decreased, respectively, barrier function (FIG. 11K-11M).

Using a similar strategy, we also tested metabolites that correlated with IEC cell type specific transcripts such as the Paneth cell gene Lyz1 (FIG. 11N). Metabolites such as MNA that positively correlated with enterocyte genes showed negative correlation with goblet and Paneth cell genes (FIG. 11O). Using the same enteroid-based approach, we found that MNA, IPA, IAM, and CARN reduced goblet and Paneth cell transcripts as well as cell numbers (FIG. 11P-11R). These results validated at physiological level that our identified LGG-related metabolites regulate IEC differentiation and function.

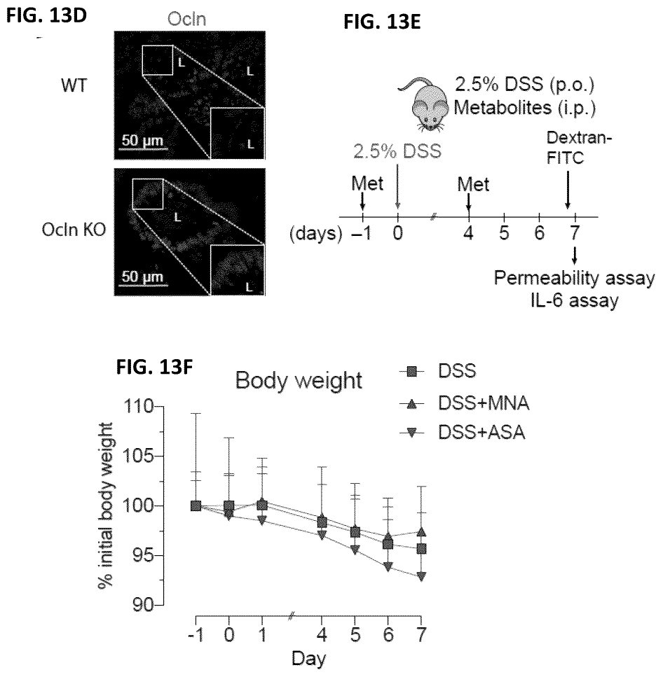

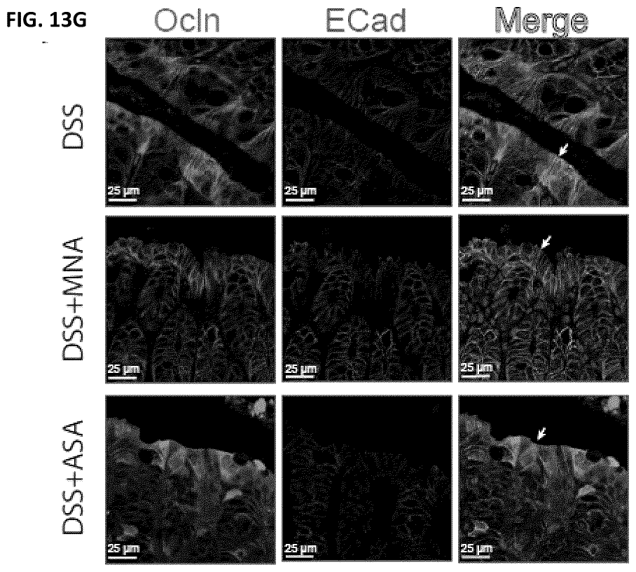

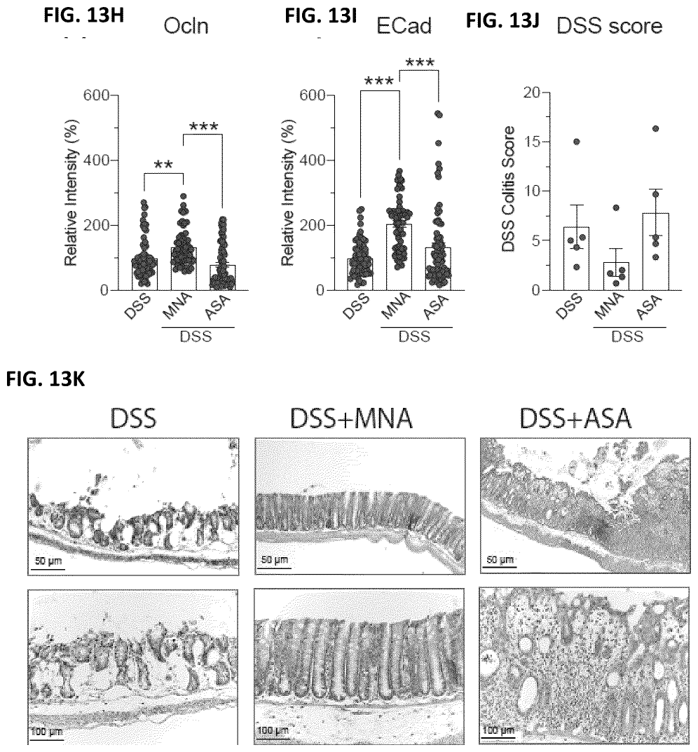

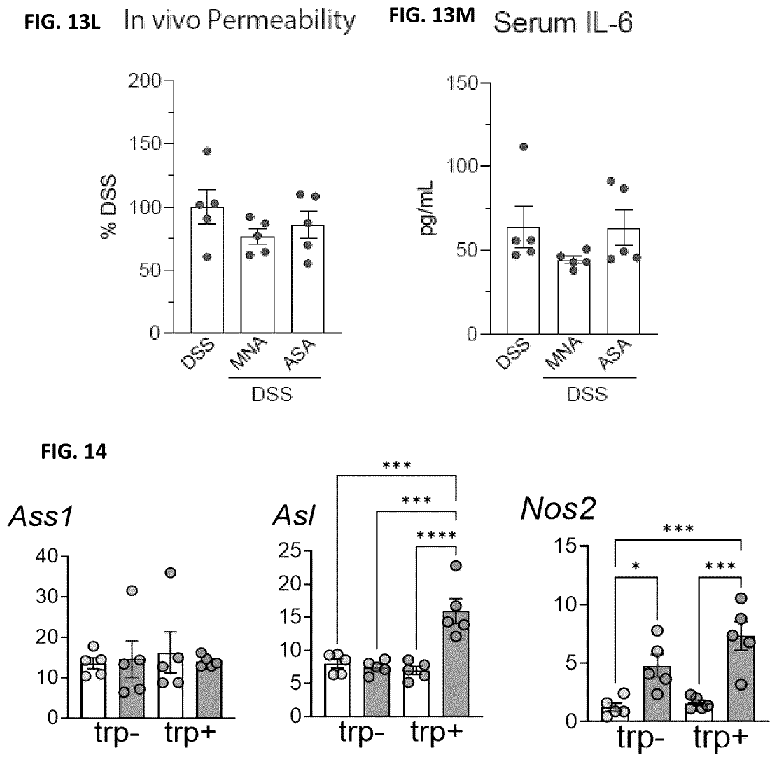

The above serum metabolites (FIG. 11I-11K) increased both Ocln expression and barrier function. Therefore, we evaluated the causal relationship whether the enhanced barrier function was dependent on TJ genes such as Ocln. We grew enteroids from Ocln KO mice (Oclnfl/fl− Vil/CRE ERT2) and subjected them to the similar experiments (FIG. 13A). Ocln KO enteroids with and without LPS showed nearly 500% greater permeability than WT control (FIG. 13B, 13C). Ocln deletion was confirmed by immunostaining (FIG. 13D). IPA did not reduce the permeability in Ocln KO enteroids, indicating that IPA's barrier enhancing effect requires Ocln.

To test if these barrier-regulating metabolites indeed modulate TJ gene expression and intestinal permeability in vivo, we investigated their effects in mice that were subjected to 2.5% DSS water treatment (FIG. 13E). Before and after DSS water treatment, we administered individual metabolites to different cohorts of mice through intraperitoneal injections. Although no significant change was observed in overall body weight among groups, mice treated with DSS plus ASA showed a greater tendency to lose weight compared with mice treated with DSS alone (FIG. 13F). MNA-treated mice demonstrated a significantly elevated Ocln expression in the colonic epithelial glands, in particular the surface epithelial cells (FIG. 13G-13H). In contrast to MNA-treated mice, ASA treated mice showed a reduction in Ocln expression (FIG. 13G-13H). E-cad abundance was significantly increased by MNA treatment and appeared sharply immunolocalized at lateral junctions (FIG. 13G, 13I). However, E-cad appeared dispersed in DSS− and DSS+ASA-treated mouse colonic epithelial cells (FIG. 13G). This data indicates that barrier gene disruption present in the colitis model was prevented during treatment with MNA, but exacerbated with ASA. Mice treated with MNA also showed improved pathology score judged from epithelial cell disruption and immune cell infiltration (FIG. 13J, 13K), decreased intestinal permeability (FIG. 13L) and serum IL-6 level (FIG. 13M) indicative of an overall ameliorated epithelial damages.

Each of the four groups was further assessed for their levels of arginine-succinate (ASL) (FIG. 14).

Moreover, levels of Argininosuccinate synthase (ASS1), and nitric oxide synthase (NOS2) were analyzed in untreated mice and mice treated with LGG (shaded bars). Both groups were fed a trp− or trp+ diet. Levels of ASS1 remained approximately the same across all four groups. NOS2 levels increased in both groups treated with LGG while ASL levels increased in mice treated with LGG and fed a trp+ diet. (FIG. 14) Levels of ASL in the other three groups was approximately the same. Thus, healthy mice monocolonized with LGG, (LGG TRP) had increased levels of ASL when compared the other groups.

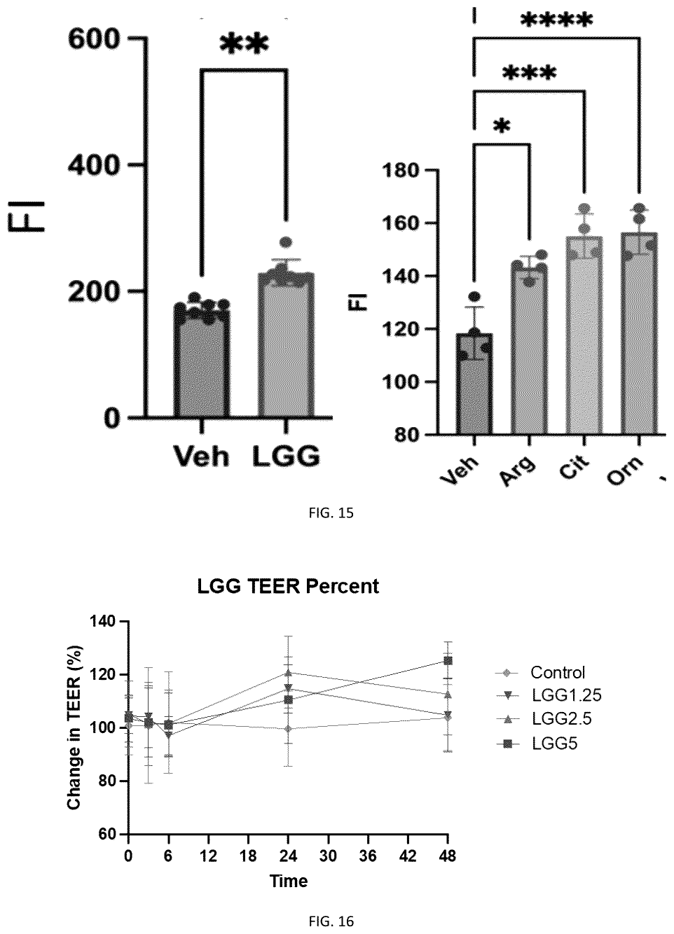

Lastly, NOS2 activity was analyzed in cells treated with an LGG supernatant (orange bar, FIG. 15). The supernatant increases NO production (represented as FI or fluorescence intensity) in polarized human Caco-2-BBE cells (left panel). This indicates that metabolites from LGG and not LGG itself mediate the effect. Additionally, NO production increases in vitro in polarized Caco-2-BBE cells (right panel) when only LGG metabolites such as arginine, ornithine or citrulline are provided to these cells. These confirm that its metabolites are the effectors of LGG's beneficial impact. (FIG. 15A-15B)

### DISCUSSION

Although worldwide consumption of probiotics has increased and now generate an industry estimated at $57 billion in 2022 (Padmanabhan, 2016), no strain has yet been approved by the FDA to treat a disease or medical condition. Increased understanding of the mechanisms of widely used strains such as LGG is necessary to improve the utility of probiotic bacteria (Preidis et al., 2020; Segers and Lebeer, 2014). However, no study has determined the interrelated impact of LGG on host metabolome and intestinal transcriptome. We have formulated a method to link potential metabolites to LGG's specific physiological effects then demonstrated that LGG interacts with dietary trp to specifically impact transcripts regulating TJ, epithelial permeability, enterocyte differentiation and function. We were able to uncover and prioritize LGG metabolites that affect intestinal epithelial TJ genes.

Although in patients and a variety of conventionally-housed animal models, LGG supplementation appeared to benefit intestinal epithelial barrier function by increasing TJ gene expression (Bajaj et al., 2014; Chen et al., 2016; Francavilla et al., 2010; Mao et al., 2016; Ritze et al., 2014; Yoshifuji et al., 2016; Zhao et al., 2015), many of these studies also found LGG-induced alterations in gut microbiota, which obscured the specific contribution from LGG. This is a notable concern, as in monocolonized pigs, LGG decreased levels of several TJ proteins in one study while had no effect on TJ gene expression in another (Liu et al., 2013; Splichalova et al., 2019). However, LGG increased TJ expression in conventionally-raised pigs (Mao et al., 2016; Zhao et al., 2015). Thus, the data presented herein further characterize the confounding role of commensal bacteria in LGG-supplemented conventional animal models by providing clarity to the LGG-specific beneficial effects.

Using our newly developed deep-learning algorithm METRCA, we discovered positive or negative correlations of specific LGG-related luminal and serum metabolites with key intestinal transcriptomic programs such as TJ and barrier function. We validated ex vivo and in vivo the top metabolites that are predicted to enhance or disrupt barrier functions. Thus, METRCA creates a predictive method that helps the assessment of a specific metabolite's relationship with any given intestinal transcriptional pathway. METRCA can be utilized to reveal potential beneficial or harmful metabolites with certain diseases. A good example is fecal sorbitol catabolized by LGG and other L. rhamnosus strains (Jiang et al., 2018; Kim et al., 2021). Sorbitol always is found in low concentrations in the feces of LGG-monoassociated (this study) or -supplemented mice (Kim et al., 2021). LGG catabolismof sorbitol is beneficial, as this sugar alcohol is a common additive in numerous foods acting as a strong laxative when inappropriate luminal levels accumulate.

The difficulty in distinguishing host versus microbe derived metabolites present a challenge in functional metabolomic study of host-microbe-dietary interaction. No study to our knowledge has delineated the contribution of LGG-dietary trp interaction to host metabolome and intestinal transcriptome. We found that LGG regulates specific sets of trp and non-trp metabolites in the presence or absence of dietary trp. Our observed effects of several indole derivatives were consistent with their reported function in repairing intestinal epithelial barrier function reported in Caco2 and HCT-8 cell lines, and in conventionally-housed animal models (Bansal et al., 2010; Li et al., 2021; Puccetti et al., 2021; Scott et al., 2020; Yoshifuji et al., 2016). Orally-administered indoles alleviated DSS colitis-induced gut permeability in GF and conventional mice (Scott et al., 2020; Shimada et al., 2013). Because of their hydrophobicity, indoles are readily absorbed. Our metabolomic study uncovered distinct indole metabolite profiles in luminal and serum compartments, as well as the differential ileal gene regulation by the same metabolites, such as IAM, indole-3-carboxyaldehyde, or uric acid, depending on their localization.

The finding that LGG suppresses certain barrier-disrupting metabolites is especially interesting. Among these TJ negative correlating metabolites are serum ASA, uric acid, leucic acid phenyllactic acid, and taurine. We found that ASA is a potent barrier disruptor ex vivo and in vivo, a finding that has not been reported previously. Serum arginine-succinate is produced in the mammalian liver as part of the urea cycle and arginine metabolism (Uyanga et al., 2021). Patients with Chronic Fatigue Syndrome typically associated with a leaky gut (Maes and Leunis, 2008) have high levels of ASA (Lupo et al., 2021). Our result indicated that a potential negative impact of gut dysbiosis on the gut barrier may attribute to production of gut-disruptive metabolites such as ASA. Thus, these targeted metabolites may be assessed as a biomarker for selective patients who will be benefited from probiotic intervention. Taken together, our study demonstrated a dietarily regulated LGG effect in modulating intestinal epithelial differentiation and function that can be mediated by metabolites.

### REFERENCES

Bajaj, J. S., Heuman, D. M., Hylemon, P. B., Sanyal, A. J., Puri, P., Sterling, R. K., Luketic, V., Stravitz, R. T., Siddiqui, M. S., Fuchs, M., et al. (2014). Randomised clinical trial: Lactobacillus GG modulates gut microbiome, metabolome and endotoxemia in patients with cirrhosis. Aliment Pharmacol Ther 39, 1113-1125.
Bansal, T., Alaniz, R. C., Wood, T. K., and Jayaraman, A. (2010). The bacterial signal indole increases epithelial-cell tight-junction resistance and attenuates indicators of inflammation. Proc Natl Acad Sci USA 107, 228-233.
Boldyrev, A. A., Aldini, G., and Derave, W. (2013). Physiology and Pathophysiology of Carnosine. Physiological Reviews 93, 1803-1845.
Capurso, L. (2019). Thirty Years of Lactobacillus rhamnosus GG: A Review. J Clin Gastroenterol 53 Suppl 1, S1-s41.
Ceapa, C., Davids, M., Ritari, J., Lambert, J., Wels, M., Douillard, F. P., Smokvina, T., de Vos, W. M., Knol, J., and Kleerebezem, M. (2016). The Variable Regions of Lactobacillus rhamnosus Genomes Reveal the Dynamic Evolution of Metabolic and Host-Adaptation Repertoires. Genome Biol Evol 8, 1889-1905.
Chen, R. C., Xu, L. M., Du, S. J., Huang, S. S., Wu, H., Dong, J. J., Huang, J. R., Wang, X. D., Feng, W. K., and Chen, Y. P. (2016). Lactobacillus rhamnosus GG supernatant promotes intestinal barrier function, balances Treg and TH17 cells and ameliorates hepatic injury in a mouse model of chronic-binge alcohol feeding. Toxicol Lett 241, 103-110.
ClinicalTrials.gov (2022). ClinicalTrials.gov (National Library of Medicine).
Das, S., Yu, S., Sakamori, R., Vedula, P., Feng, Q., Flores, J., Hoffman, A., Fu, J., Stypulkowski, E., Rodriguez, A., et al. (2015). Rab8a vesicles regulate Wnt ligand delivery and Paneth cell maturation at the intestinal stem cell niche. Development 142, 2147-2162.
Erez, A., Nagamani, S. C., and Lee, B. (2011). Argininosuccinate lyase deficiency-argininosuccinic aciduria and beyond. Am J Med Genet C Semin Med Genet 157C, 45-53.
Fontaine, C. A., Skorupski, A. M., Vowles, C. J., Anderson, N. E., Poe, S. A., and Eaton, K. A. (2015). How free of germs is germ-free? Detection of bacterial contamination in a germ free mouse unit. Gut Microbes 6, 225-233.
Francavilla, R., Miniello, V., Magista, A. M., De Canio, A., Bucci, N., Gagliardi, F., Lionetti, E., Castellaneta, S., Polimeno, L., Peccarisi, L., et al. (2010). A randomized controlled trial of Lactobacillus GG in children with functional abdominal pain. Pediatrics 126, e1445-1452.
Gorbach, S. L., Doron, S. I., and Magro, F. (2017). Lactobacillus rhamnosus GG. In The Microbiota in Gastrointestinal Physiology, M. H. Floch, R. Y., and W. A. Walker, eds. (New York: Elsevier), pp. 79-89.
Hollander, D., and Kaunitz, J. D. (2020). The “Leaky Gut”: Tight Junctions but Loose Associations? Dig Dis Sci 65, 1277-1287.
Jang, H. R., Park, H.-J., Kang, D., Chung, H., Nam, M. H., Lee, Y., Park, J.-H., and Lee, H.-Y. (2019). A protective mechanism of probiotic Lactobacillus against hepatic steatosis via reducing host intestinal fatty acid absorption. Exp Mol Med 51, 1-14.
Jiang, Q., Kainulainen, V., Stamatova, I., Korpela, R., and Meurman, J. H. (2018). Lactobacillus rhamnosus GG in Experimental Oral Biofilms Exposed to Different Carbohydrate Sources. Caries Res 52, 220-229.
Keszthelyi, D., Troost, F. J., Jonkers, D. M., van Eijk, H. M., Lindsey, P. J., Dekker, J., Buurman, W. A., and Masclee, A. A. (2014). Serotonergic reinforcement of intestinal barrier function is impaired in irritable bowel syndrome. Aliment Pharmacol Ther 40, 392-402.
Kim, C. J., Kovacs-Nolan, J. A., Yang, C., Archbold, T., Fan, M. Z., and Mine, Y. (2010). l-Tryptophan exhibits therapeutic function in a porcine model of dextran sodium sulfate (DSS)-induced colitis. J Nutr Biochem 21, 468-475.
Kim, J., Balasubramanian, I., Bandyopadhyay, S., Nadler, I., Singh, R., Harlan, D., Bumber, A., He, Y., Kerkhof, L. J., Gao, N., et al. (2021). Lactobacillus rhamnosus GG modifies the metabolome of pathobionts in gnotobiotic mice. BMC Microbiol 21, 165.
Kinashi, Y., and Hase, K. (2021). Partners in Leaky Gut Syndrome: Intestinal Dysbiosis and Autoimmunity. Frontiers in Immunology 12.
Li, J., Zhang, L., Wu, T., Li, Y., Zhou, X., and Ruan, Z. (2021). Indole-3-propionic Acid Improved the Intestinal Barrier by Enhancing Epithelial Barrier and Mucus Barrier. J Agric Food Chem 69, 1487-1495.
Li, Y. T., Xu, H., Ye, J. Z., Wu, W. R., Shi, D., Fang, D. Q., Liu, Y., and Li, L. J. (2019). Efficacy of Lactobacillus rhamnosus GG in treatment of acute pediatric diarrhea: A systematic review with meta-analysis. World J Gastroenterol 25, 4999-5016.
Liu, F., Li, G., Wen, K., Wu, S., Zhang, Y., Bui, T., Yang, X., Kocher, J., Sun, J., Jortner, B., et al. (2013). Lactobacillus rhamnosus GG on rotavirus-induced injury of ileal epithelium in gnotobiotic pigs. J Pediatr Gastroenterol Nutr 57, 750-758.
Liu, M., Li, L., Chu, J., Zhu, B., Zhang, Q., Yin, X., Jiang, W., Dai, G., Ju, W., Wang, Z., et al. (2015). Serum N (1)-Methylnicotinamide Is Associated With Obesity and Diabetes in Chinese. J Clin Endocrinol Metab 100, 3112-3117.
Liu, X., Jin, G., Tang, Q., Huang, S., Zhang, Y., Sun, Y., Liu, T., Guo, Z., Yang, C., Wang, B., et al. (2022). Early life Lactobacillus rhamnosus GG colonisation inhibits intestinal tumour formation. Br J Cancer 126, 1421-1431.
Lupo, G. F. D., Rocchetti, G., Lucini, L., Lorusso, L., Manara, E., Bertelli, M., Puglisi, E., and Capelli, E. (2021). Potential role of microbiome in Chronic Fatigue Syndrome/Myalgic Encephalomyelits (CFS/ME). Scientific Reports 11, 7043.
Lyu, L., Zhou, X., Zhang, M., Liu, L., Liu, T., Niu, H., Wu, Y., Liang, C., Han, X., and Zhang, L. (2022). Lactobacillus derived from breast milk facilitates intestinal development in IUGR rats. Journal of Applied Microbiology n/a.
Maes, M., and Leunis, J. C. (2008). Normalization of leaky gut in chronic fatigue syndrome (CFS) is accompanied by a clinical improvement: effects of age, duration of illness and the translocation of LPS from gram-negative bacteria. Neuro Endocrinol Lett 29, 902-910.
Mantegazza, C., Molinari, P., D'Auria, E., Sonnino, M., Morelli, L., and Zuccotti, G. V. (2018). Probiotics and antibiotic-associated diarrhea in children: A review and new evidence on Lactobacillus rhamnosus GG during and after antibiotic treatment. Pharmacol Res 128, 63-72.
Mao, X., Gu, C., Hu, H., Tang, J., Chen, D., Yu, B., He, J., Yu, J., Luo, J., and Tian, G. (2016). Dietary Lactobacillus rhamnosus GG Supplementation Improves the Mucosal Barrier Function in the Intestine of Weaned Piglets Challenged by Porcine Rotavirus. PLOS One 11, e0146312.
Mardinoglu, A., Shoaie, S., Bergentall, M., Ghaffari, P., Zhang, C., Larsson, E., Bäckhed, F., and Nielsen, J. (2015). The gut microbiota modulates host amino acid and glutathione metabolism in mice. Mol Syst Biol 11, 834.
McFarland, L. V., Evans, C. T., and Goldstein, E. J. C. (2018). Strain-Specificity and Disease-Specificity of Probiotic Efficacy: A Systematic Review and Meta-Analysis. Front Med (Lausanne) 5, 124.
Mehandru, S., and Colombel, J. F. (2021). The intestinal barrier, an arbitrator turned provocateur in IBD. Nat Rev Gastroenterol Hepatol 18, 83-84.
Melamud, E., Vastag, L., and Rabinowitz, J. D. (2010). Metabolomic analysis and visualization engine for LC-MS data. Anal Chem 82, 9818-9826.
Moon, C., Baldridge, M. T., Wallace, M. A., D, C. A., Burnham, Virgin, H. W., and Stappenbeck, T. S. (2015). Vertically transmitted faecal IgA levels determine extra-chromosomal phenotypic variation. Nature 521, 90-93.
Mu, Q., Kirby, J., Reilly, C. M., and Luo, X. M. (2017). Leaky Gut As a Danger Signal for Autoimmune Diseases. Front Immunol 8, 598.
Odenwald, M. A., Choi, W., Buckley, A., Shashikanth, N., Joseph, N. E., Wang, Y., Warren, M. H., Buschmann, M. M., Pavlyuk, R., Hildebrand, J., et al. (2017). ZO-1 interactions with F-actin and occludin direct epithelial polarization and single lumen specification in 3D culture. J Cell Sci 130, 243-259.
Padmanabhan, S. (2016). Probiotics Market to Reach $57.4 Billion, Globally, by 2022. In Probiotics Market-Opportunities and Forecasts 2014-2022 (Portland, Oregon: Allied Analytics LLP).
Pearce, S. C., Al-Jawadi, A., Kishida, K., Yu, S., Hu, M., Fritzky, L. F., Edelblum, K. L., Gao, N., and Ferraris, R. P. (2018). Marked differences in tight junction composition and macromolecular permeability among different intestinal cell types. BMC Biol 16, 19.
Preidis, G. A., Weizman, A. V., Kashyap, P. C., and Morgan, R. L. (2020). AGA Technical Review on the Role of Probiotics in the Management of Gastrointestinal Disorders. Gastroenterology 159, 708-738 e704.
Puccetti, M., Pariano, M., Borghi, M., Barola, C., Moretti, S., Galarini, R., Mosci, P., Ricci, M., Costantini, C., and Giovagnoli, S. (2021). Enteric formulated indole-3-carboxaldehyde targets the aryl hydrocarbon receptor for protection in a murine model of metabolic syndrome. Int J Pharm 602, 120610.
Ritze, Y., Bardos, G., Claus, A., Ehrmann, V., Bergheim, I., Schwiertz, A., and Bischoff, S. C. (2014). Lactobacillus rhamnosus GG protects against non-alcoholic fatty liver disease in mice. PLOS One 9, e80169.
Roager, H. M., and Licht, T. R. (2018). Microbial tryptophan catabolites in health and disease. Nat Commun 9, 3294.
Scott, S. A., Fu, J., and Chang, P. V. (2020). Microbial tryptophan metabolites regulate gut barrier function via the aryl hydrocarbon receptor. Proc Natl Acad Sci USA 117, 19376-19387.
Segers, M. E., and Lebeer, S. (2014). Towards a better understanding of Lactobacillus rhamnosus GG—host interactions. Microb Cell Fact 13 Suppl 1, S7.
Sentongo, T. A., Cohran, V., Korff, S., Sullivan, C., Iyer, K., and Zheng, X. (2008). Intestinal permeability and effects of Lactobacillus rhamnosus therapy in children with short bowel syndrome. J Pediatr Gastroenterol Nutr 46, 41-47.
Shen, X., Liu, L., Peek, R. M., Acra, S. A., Moore, D. J., Wilson, K. T., He, F., Polk, D. B., and Yan,
F. (2018). Supplementation of p40, a Lactobacillus rhamnosus GG-derived protein, in early life promotes epidermal growth factor receptor-dependent intestinal development and long-term health outcomes. Mucosal Immunol 11, 1316-1328.
Shimada, Y., Kinoshita, M., Harada, K., Mizutani, M., Masahata, K., Kayama, H., and Takeda, K. (2013). Commensal bacteria-dependent indole production enhances epithelial barrier function in the colon. PLOS One 8, e80604.
Silverman, G. J., Azzouz, D. F., and Alekseyenko, A. V. (2019). Systemic Lupus Erythematosus and dysbiosis in the microbiome: cause or effect or both? Curr Opin Immunol 61, 80-85.
Sommer, F., Nookaew, I., Sommer, N., Fogelstrand, P., and Bäckhed, F. (2015). Site-specific programming of the host epithelial transcriptome by the gut microbiota. Genome Biol 16, 62.
Splichalova, A., Jenistova, V., Splichalova, Z., and Splichal, I. (2019). Colonization of preterm gnotobiotic piglets with probiotic Lactobacillus rhamnosus GG and its interference with Salmonella Typhimurium. Clinical & Experimental Immunology 195, 381-394.
Szajewska, H., and Kołodziej, M. (2015). Systematic review with meta-analysis: Lactobacillus rhamnosus GG in the prevention of antibiotic-associated diarrhoea in children and adults. Aliment Pharmacol Ther 42, 1149-1157.
Turpin, W., Lee, S. H., Raygoza Garay, J. A., Madsen, K. L., Meddings, J. B., Bedrani, L., Power, N., Espin-Garcia, O., Xu, W., Smith, M. I., et al. (2020). Increased Intestinal Permeability Is Associated With Later Development of Crohn's Disease. Gastroenterology 159, 2092-2100 e2095.
Uyanga, V. A., Amevor, F. K., Liu, M., Cui, Z., Zhao, X., and Lin, H. (2021). Potential Implications of Citrulline and Quercetin on Gut Functioning of Monogastric Animals and Humans: A Comprehensive Review. Nutrients 13, 3782.
Vyhlidalova, B., Krasulova, K., Pecinkova, P., Marcalikova, A., Vrzal, R., Zemankova, L., Vanco, J., Travnicek, Z., Vondracek, J., Karasova, M., et al. (2020). Gut Microbial Catabolites of Tryptophan Are Ligands and Agonists of the Aryl Hydrocarbon Receptor: A Detailed Characterization. Int J Mol Sci 21.
Wang, Y.-F., Hsu, Y.-J., Wu, H.-F., Lee, G.-L., Yang, Y.-S., Wu, J.-Y., Yet, S.-F., Wu, K. K., and Kuo, C.-C. (2016). Endothelium-Derived 5-Methoxytryptophan Is a Circulating Anti-Inflammatory Molecule That Blocks Systemic Inflammation. Circulation Research 119, 222-236.
Yan, F., Liu, L., Cao, H., Moore, D. J., Washington, M. K., Wang, B., Peek, R. M., Acra, S. A., and Polk, D. B. (2017). Neonatal colonization of mice with LGG promotes intestinal development and decreases susceptibility to colitis in adulthood. Mucosal Immunol 10, 117-127.
Yoo, J. H., and Donowitz, M. (2019). Intestinal enteroids/organoids: A novel platform for drug discovery in inflammatory bowel diseases. World J Gastroenterol 25, 4125-4147.
Yoshifuji, A., Wakino, S., Irie, J., Tajima, T., Hasegawa, K., Kanda, T., Tokuyama, H., Hayashi, K., and Itoh, H. (2016). Gut Lactobacillus protects against the progression of renal damage by modulating the gut environment in rats. Nephrol Dial Transplant 31, 401-412.
Yusufu, I., Ding, K., Smith, K., Wankhade, U. D., Sahay, B., Patterson, G. T., Pacholczyk, R., Adusumilli, S., Hamrick, M. W., Hill, W. D., et al. (2021). A Tryptophan-Deficient Diet Induces Gut Microbiota Dysbiosis and Increases Systemic Inflammation in Aged Mice. Int J Mol Sci 22.
Zelante, T., Iannitti, R. G., Cunha, C., De Luca, A., Giovannini, G., Pieraccini, G., Zecchi, R., D'Angelo, C., Massi-Benedetti, C., Fallarino, F., et al. (2013). Tryptophan catabolites from microbiota engage aryl hydrocarbon receptor and balance mucosal reactivity via interleukin-22. Immunity 39, 372-385.
Zhao, H., Zhao, C., Dong, Y., Zhang, M., Wang, Y., Li, F., Li, X., McClain, C., Yang, S., and Feng, W. (2015). Inhibition of miR 122a by Lactobacillus rhamnosus GG culture supernatant increases intestinal occludin expression and protects mice from alcoholic liver disease. Toxicol Lett 234, 194-200.
Zheng, J., Wittouck, S., Salvetti, E., Franz, C., Harris, H. M. B., Mattarelli, P., O'Toole, P. W., Pot, B., Vandamme, P., Walter, J., et al. (2020). A taxonomic note on the genus Lactobacillus: Description of 23 novel genera, emended description of the genus Lactobacillus Beijerinck 1901, and union of Lactobacillus and Leuconostocaceae. Int J Syst Evol Microbiol 70, 2782-2858.

### Example 2: Nutraceutical Formulations

Leaky gut syndrome is an increasingly recognized medical condition associated with a large array of mostly autoimmune diseases, and an impaired intestinal barrier precedes and even predicts clinical diagnosis of some of these diseases, particularly type 1 diabetes and inflammatory bowel disease (IBD) by several years. Described herein is the use of specific metabolites identified from interactions among probiotic gut bacteria, host and host diet, to reduce these leaks thereby either preventing the onset or reducing the severity of these diseases. The identified beneficial metabolites from probiotic bacteria now have linked via rigorous experiments to directly impact the gut barrier.

We used an innovative metabolome-transcriptome correlation analysis of samples from mice monocolonized with Lactobacillus rhamnosus GG (LGG), and discovered specific serum and intestinal metabolites that promote or impair barrier function. (See Tables 2 and 3) In addition to enhancing the production of beneficial metabolites, LGG, in conjunction with dietary tryptophan, significantly reduces deleterious metabolites that reduce expression of barrier genes and impair barrier function in and ex vivo. Our results demonstrate that LGG synergizes with a key dietary component to regulate gut epithelial differentiation and permeability by altering the abundances of barrier-modulating metabolites.

The metabolites from Example 1 were administered ex vivo and analyzed using fluorescence microscopy. Indole acetamide (IAM), methynicotinamide (MNA) and carnosine (CARN) prevented disruption of the gut barrier (less green, top row). Conversely, the negatively correlated metabolite arginino-succinate disrupted the gut (not shown). (FIG. 11M). Further analysis of these metabolites showed that IAM, MNA, CARN, and indole-propionic acid (IPA) also prevented increases in gut barrier permeability. Conversely, ASA did not. (FIG. 11K-11L).

When a barrier disruptor is administered, ex vivo expression of the Ocln gene is decreased. However, administration of IPA, IAM, MNA, and CARN increased gene expression of Ocln even in the presence of the barrier disruptor. (FIG. 11J).

Our data clearly confirms in vitro the efficacy of multiple beneficial metabolites including IPA, IAM, MNA, and CARN. These metabolites were further analyzed in vivo in a mouse colitis model. Mice were administered either MNA or ASA metabolites intraperitoneally prior to induction of colitis. 2.5% DSS was administered to induce mild colitis and leaky gut. After 4 days, the metabolites were administered a second time. The MNA sealed the leaky gut, as colitis mice treated with MNA, compared to colitis mice, showed improved pathology score judged from epithelial cell disruption and immune cell infiltration of histological slides, decreased intestinal permeability in vivo and reduced serum proinflammatory IL-6 levels (FIG. 13K-13M). These results are indicative of an overall amelioration of epithelial damage.

Further, our candidate metabolites prevented lipopolysaccharide (LPS)-induced increases in epithelial permeability in vitro, thus achieving treatment of leaky gut. (FIG. 11K)

To confirm that the metabolites were causing the effect on the gut barrier. LGG was cultured in a test tube, centrifuged and supernatant collected. This supernatant which has LGG metabolites but not LGG increased transepithelial resistance (strengthens the gut barrier) of Caco-2 cells. Applying LGG spent medium enhanced TEER within 24 hrs. This effect was prolonged by a higher concentration (5%, red line) (FIG. 16). An increase in TEER would indicate an enhanced gut barrier. Applying spent media from a control bacteria did not alter TEER. This indicates that isolated metabolites from LGG, and not LGG itself, also effectively improves the gut barrier. (FIG. 16)

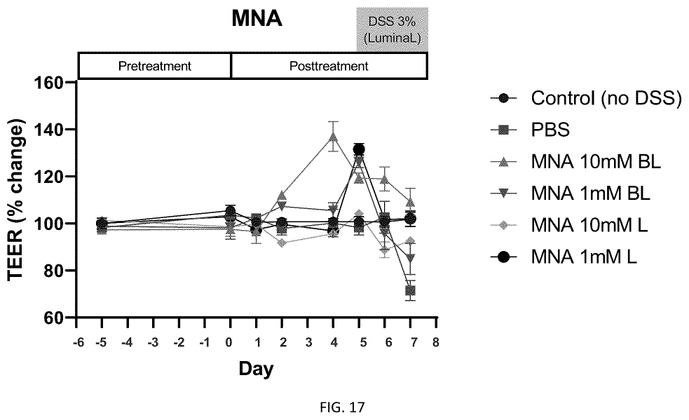

Lastly, oral administration of the MNA metabolite was analyzed. Caco2 were provided with MNA at day 0 either luminally (L, mimics oral) or basolaterally (BL, mimics intra-peritoneal) (FIG. 17). Then DSS was added to lumen to disrupt gut barrier in all treatments except control (blue circles). DSS disrupted gut barrier (red square) as shown by reduced TEER (measures barrier integrity). A high and low dose of basolateral MNA rescued Caco-2 TEER (triangles) from DSS-induced permeability. A low dose of luminal MNA prevented barrier disruption (black circles), indicating that MNA can be provided orally. Similar results were obtained if other LGG metabolite, such as IPA, was used. (FIG. 17)

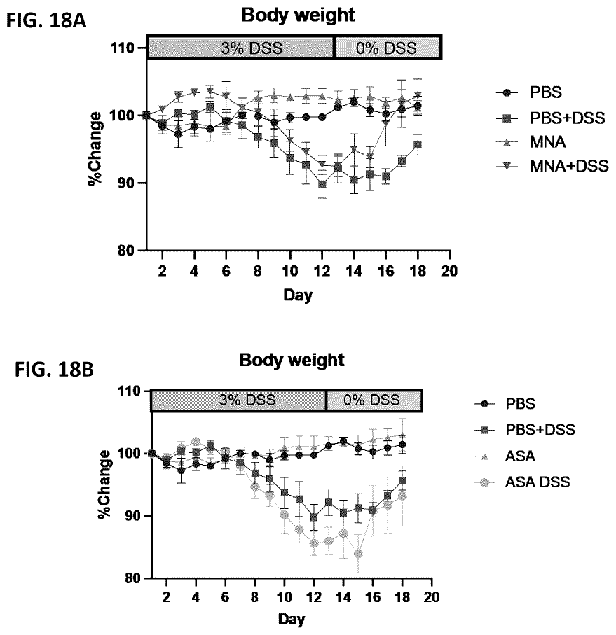

The LGG serum metabolite MNA (FIG. 18A) that is positively correlated to expression of gut barrier genes, clearly had no deleterious effect on body weight when injected alone. It dampened then accelerated the recovery of body weight in a leaky gut mouse model. In clear contrast, another LGG metabolite that is negatively correlated to barrier genes, ASA, exacerbated the effects of DSS, and significantly delayed recovery (FIG. 18B). In summary, MNA and beneficial metabolites can be used as treatments for leaky gut and associated conditions. ASA and deleterious metabolites can be used as biomarkers of leaky gut and thus indicate a condition where beneficial metabolites can be applied.

### Example 3: LLG Metabolites Methylnicotinamide (MNA) and Indoles can Prevent or Protect from Leaky Gut

Our in silico analysis of already published data on serum metabolites in 484 ulcerative colitis (UC) and 464 Crohn's disease CD) patients was compared to that of 365 healthy controls (Di′Narzo et al 2022). This analysis revealed significantly lower levels of many indoles in inflammatory bowel disease (IBD) patients. We found strong association of low serum MNA and xanthenurate (XAN) levels with increased disease severity in UC and CD, while high fecal KYN is linked with the severity of CD. MNA is the strongest negative correlator with severity of UC. These findings indicate that enhancing levels of serum MNA dampens the severity of IBD, and that consumption of MNA alone, or in a cocktail of these metabolites, is protective against leaky gut.

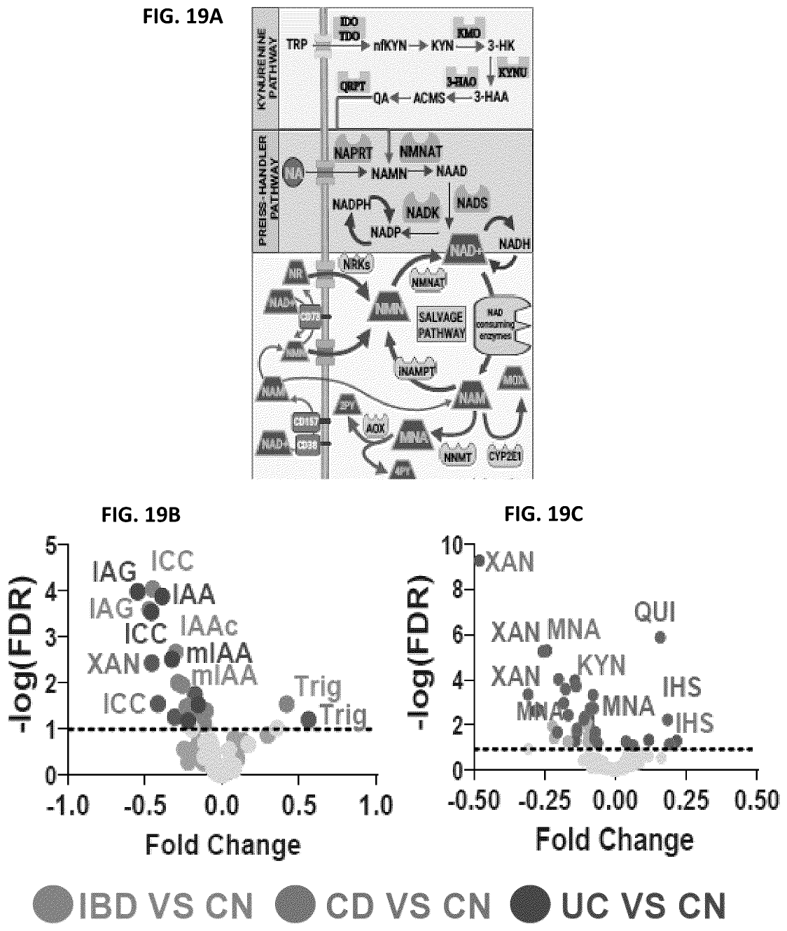

A diagram of the various indole metabolites in the pathways of dietary tryptophan is shown in FIG. 19A. In our analysis we compared levels of various indoles (which are made from tryptophan by bacteria inside human intestines) in healthy patients (CN) and patients with IBD, CD, UC. Indole acetotylcarnitine (ICC), indoleglutamine (IAG), indole acetic acid (IAA), indole-7-acetic acid (IAAc) and methyl indole-3-acetate (mIAA) all were reduced in the serum of IBD, UC and CD patients. (FIG. 19B). Levels of MNA, XAN, and KYN are also low in the serum of IBD, UC, and CD patients. (FIG. 19C) These data indicate that directly supplementing human diets with these metabolites will be protective against IBD and leaky gut.

### Example 4: MMN Dampens and/or Prevents Barrier Disruption in Human Intestinal Epithelial Cell Line Caco2

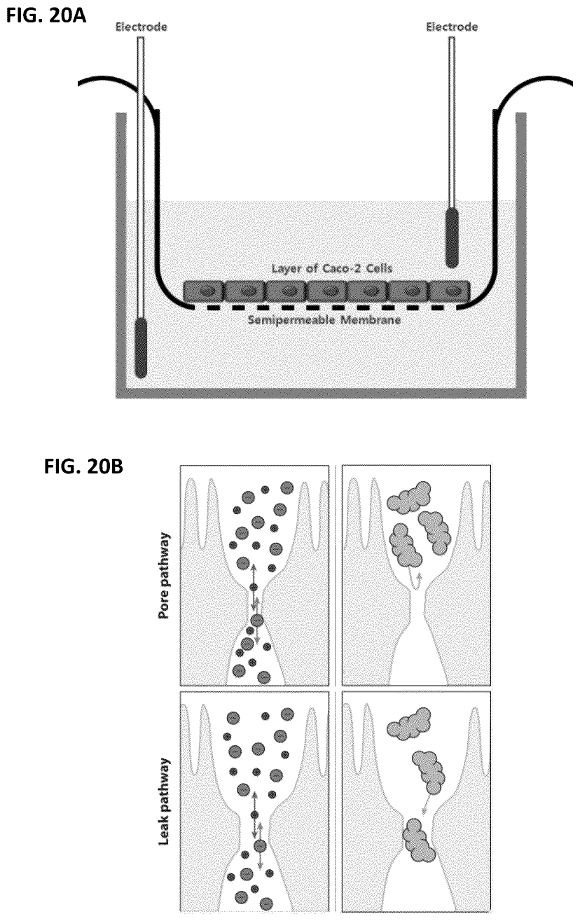

To perform in vitro screening of the metabolites effective against leaky gut, an in vitro transwell method was used. (FIG. 20A) The efficacy of these metabolites in protecting the leaky gut was analyzed in human-derived Caco-2 cells. Transepithelial electrical resistance (TEER) measurements evaluates the leakiness of the tight junction that regulates the pore (FIG. 20B, top) and leak (FIG. 20B, bottom) pathways of the gut. The higher the TEER, the less permeable the junction is to the movement of ions and small molecules. FITC-dextran is a large polysaccharide with a fluorescent moiety, typically used as a probe to assess the leakiness of the tight junction to large molecules like dextran. The faster the movement of FITC-dextran from lumen to blood, the greater the permeability.

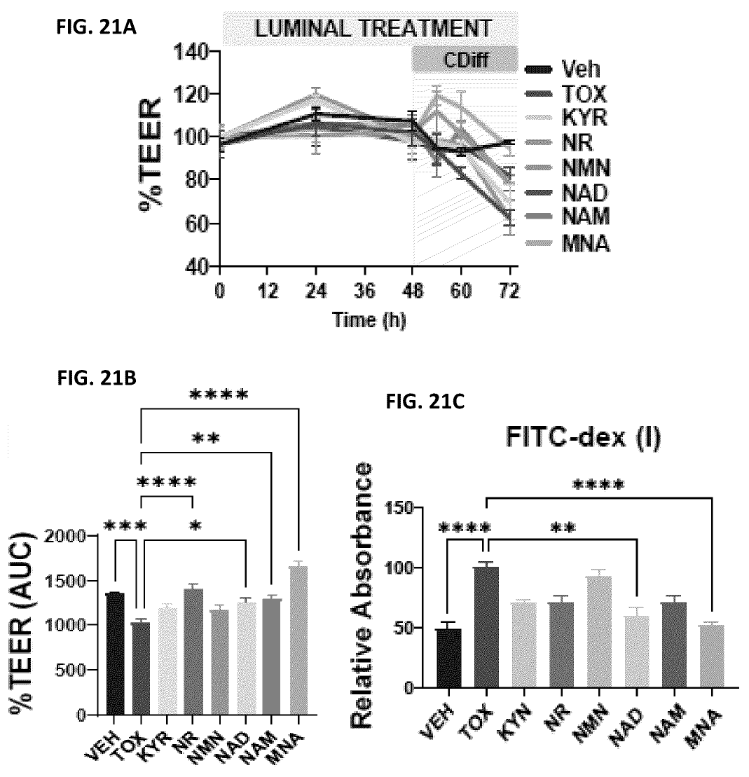

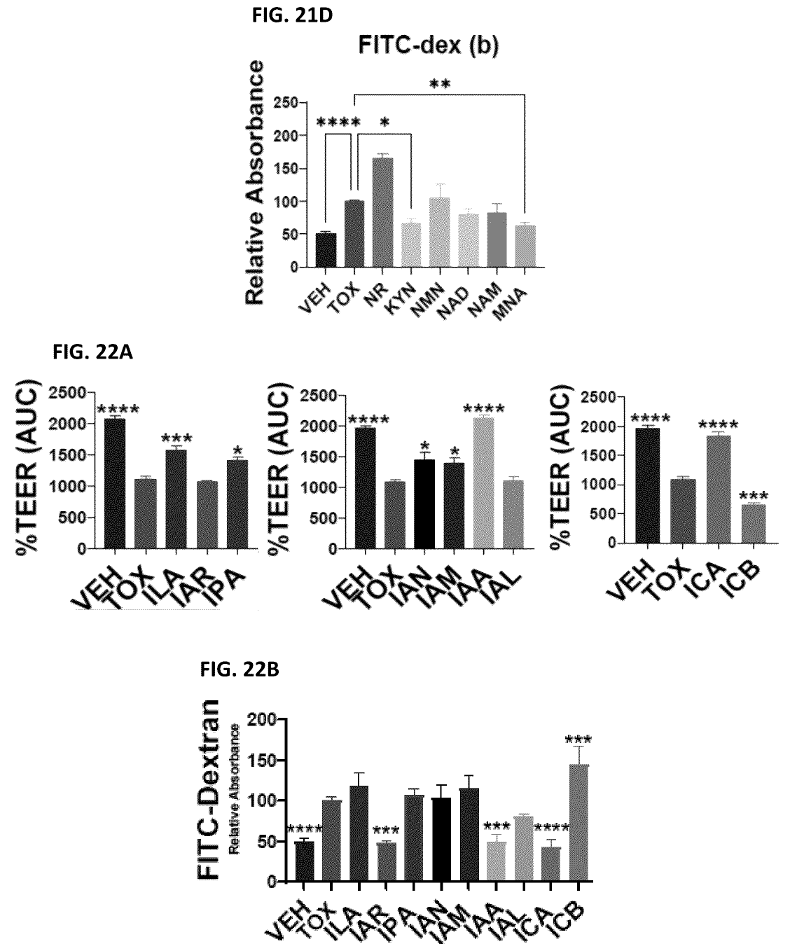

The metabolites identified in Example 3 were placed in the luminal side of the transwells and their effect on TEER was observed for 48 h. (FIG. 21A) There was no effect of these metabolites on TEER when applied by themselves. After 48 h, the barrier was disrupted by the toxin of a common “bad” bacteria Clostridium difficile (Cdiff). TEER plummeted with this toxin (TOX, red line) compared to control (Veh, blue line). With the exception of NMN, all other metabolites modestly to markedly prevented the toxin-induced disruption of the barrier. MNA was the best at preventing the toxin-induced disruption of the barrier. (FIG. 21A)

Using the data under the curve of FIG. 21A, an integrated result was presented in FIG. 21B. This shows that Veh has a high TEER, Tox has a low TEER (more permeable indicating disruption), and that MNA, NR (nicotinamide riboside), NAM (nicotinamide), and NAD all protected the cells from barrier disruption by TOX. Next, fluorescein isothiocyanate (FITC)-dextran permeability of the metabolites was analyzed. (FIG. 21C) FITC-dextran permeability was clearly increased by TOX. When applied luminally, all metabolites prevented the TOX-induced disruption in gut barrier, with MNA and NAD being the best and most significant. The FITC-dextran permeability was also measured with metabolites applied basolaterally. (FIG. 21D) When applied basolaterally, all metabolites again prevented the TOX-induced barrier disruption, with MNA and KYN being the best. These findings indicate that MNA, which is low in serum of IBD patients, is highly effective in protecting the integrity of the gut barrier.

Next the efficacy of the microbiota-derived indole metabolites in protecting the gut barrier was analyzed. C. diff toxin (TOX) clearly reduced the TEER, indicating that it disrupted the gut barrier. (FIG. 22A) Indoles are only made by gut bacteria. When our indole metabolites were applied to the lumen, indole acetic acid (IAA), indole carboxylic acid (ICA), indole lactic acid (ILA), indole propionic acid (IPA), indole acetonitrile (IAN) and indole acetamide (IAM), in that order, were clearly protective against TOX-induced decreases in TEER. Generally similar results were observed when these metabolites were applied in the basolateral compartment, except that indole carboxyaldehyde (IAL) and IAM are more effective when applied in this compartment than in the luminal. (FIG. 22A) FITC-dextran permeability was clearly increased by TOX. (FIG. 22B) When applied luminally, most metabolites prevented the TOX-induced disruption in gut barrier, with IAR, IAA, ICA and IAM being the most effective.

Severity of C. difficile toxin-induced disruption, as defined by TEER and FITC-dextran permeability (see FIG. 20), of epithelial barrier in Caco2 monolayer was either reversed or reduced in magnitude by luminal indole-acetic acid,-lactic acid,-propionic acid,-acetonitrile and -acetamide (which were abundant in the feces) as well as by basolateral indole-acetamide,-carboxyaldehyde and -lactic acid (abundant in the serum).

Toxin-induced disruption was also reversed or dampened by luminal methynicotinamide, nicotinamide riboside, and NAD, and basolateral methynicotinamide, nicotinamide mononucleotide, NAD and kynurenine. Additionally, MNA and its related metabolites have an equal, or superior, ability to repair a disrupted gut barrier when compare to several other indoles. Some indoles are already in patents as protective against leaky gut. Supplementing these indoles would improve outcomes for patients with leaky gut.

Many of the metabolites that we found correlated to barrier genes from the serum side. These metabolites can also regulate the gut barrier functionally from the luminal side. This is important because oral supplements require less regulation and cost than injectable supplements.

### Example 5: MNA Dampens Colitis-Induced Inflammation, Barrier Disruption & Tissue Damage in DDS Model

Administration of DSS induces experimental colitis and disrupts gut barrier in vivo in animal models. No significant changes in body weight were previously observed in the non-colitis groups, but the DSS-treated group declined markedly after 8 days (FIG. 18A). Remarkably, during the recovery phase, the MNA-treated DSS colitis mice exhibited a robust and faster recovery, reaching 100% of initial body weight within 6 days, in contrast to the PBS-treated DSS-colitis mice. This supports the remarkable efficacy of MNA in promoting colonic healing.

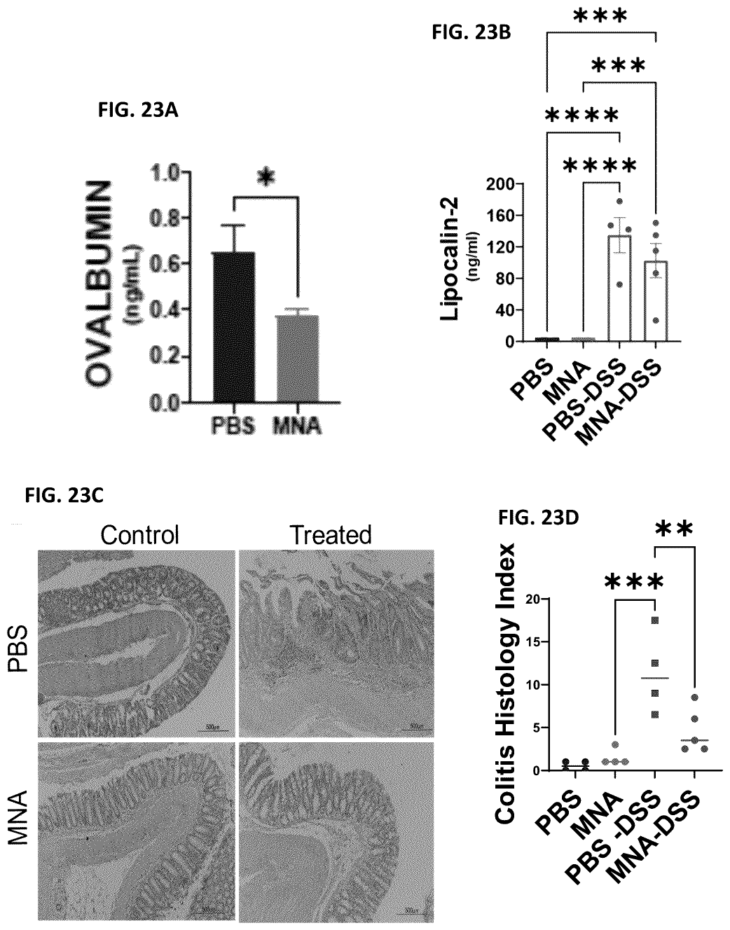

MNA is able to dampen the effects of DSS-induced leaky gut colitis on body weight by reducing inflammation, barrier disruption and tissue damage (FIG. 23). Exogenous MNA administration was assessed by plasma ovalbumin levels after oral ovalbumin gavage—an accepted method to determine permeability of the gut paracellular pathway. The exogenous MNA, when administered via i.p. injection, significantly increased barrier function by 2-fold when compared to PBS treated mice. (FIG. 23A). Moreover, MNA treatment of DSS mice (MNA-DSS) reduced fecal lipocalin (a classical biomarker of inflammation) levels compared to PBS treated DSS mice (PBS-DSS), supporting MNA-mediated mucosal protection against inflammation. (FIG. 23B) Histology sections of mice treated with or without DSS and PBS or MNA were also obtained. These sections depict tissue damage in the DDS-treated animals. The DDS tissue damage was alleviated by MNA administration. (FIG. 23C). Sections from several mice were also examined by a blinded observer and scored for colitis index. The colitis score increased markedly in PBS-DSS, but MNA protected the tissue from damage even with DSS. (FIG. 23D)

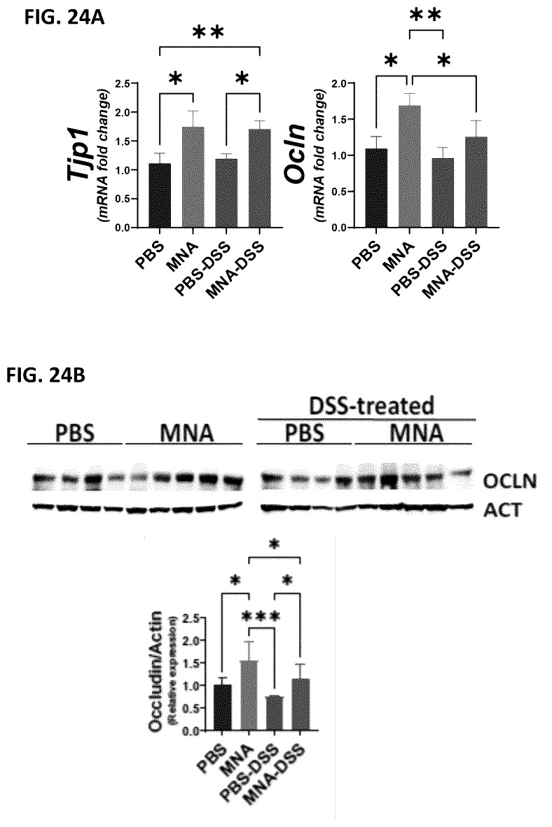

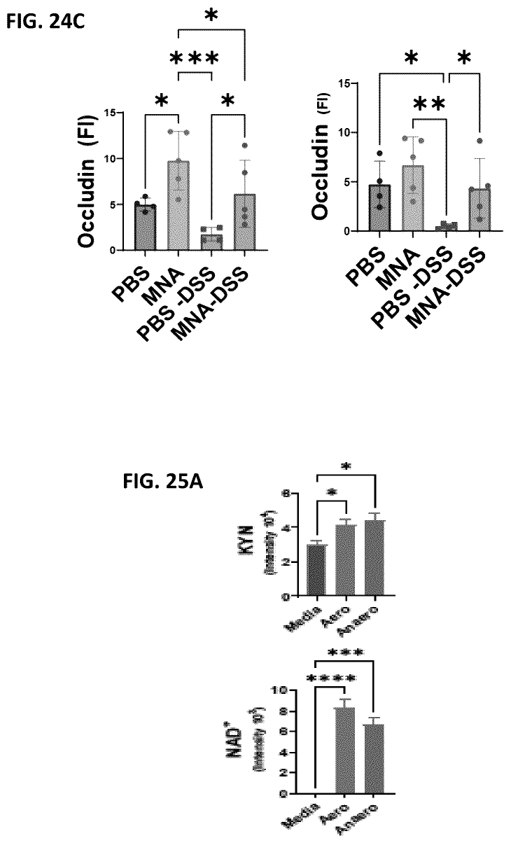

Our analysis further indicates that administration of MNA can increase mRNA and protein levels of important tight junction components to seal and protect the gut barrier from insults, inflammation, barrier disruption and tissue damage. Analysis of the mRNA levels of two important tight junction components in the ileum, zonula occludens (Tjp1) and occludin (Ocln), show that MNA protects against DDS-induced gut permeability by increasing the mRNA levels to normal. (FIG. 24A). Similar results were observed for protein levels, analyzed by Western blot, and quantified protein levels of occludin. (FIG. 24B) Using immunofluoresce antibody staining, quantified levels of occludin in the ileum and colon were also analyzed. (FIG. 24C)

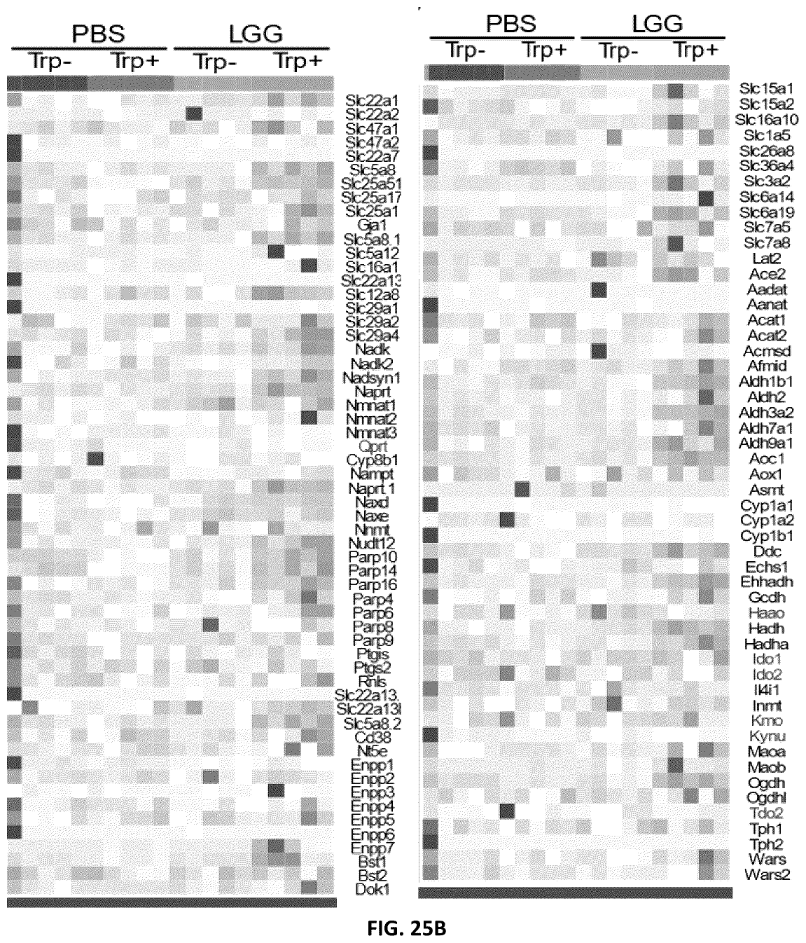

Additionally, we observed that LGG in aerobic and anaerobic culture by itself can synthesize NAD and the trp metabolite kynurenine (KYN). This can then be converted by the host to increase endogenous supplies of MNA and related metabolites. LGG makes these MNA precursors by upregulating trp and vitamin B3 metabolism pathways. (FIG. 25A)

Genes involved in vitamin B3/nicotinamide (left panel) and in tryptophan metabolism pathways (right panel) are increased in LGG trp+ mice. (FIG. 25B) This indicates that the LGG-made precursors in the lumen can be transported and moved into the host cell. This conclusion is supported by the increased levels of tryptophan-nicotinamide enzymes. This indicates LGG with dietary tryptophan robustly promotes gut barrier genes and is protective against leaky gut by increasing vitamin B3 precursors in the lumen, thereby increasing vitamin B3 metabolites, particularly MNA, in the host.

### Example 5: Argininosuccinate (ASA) as an IBD Biomarker

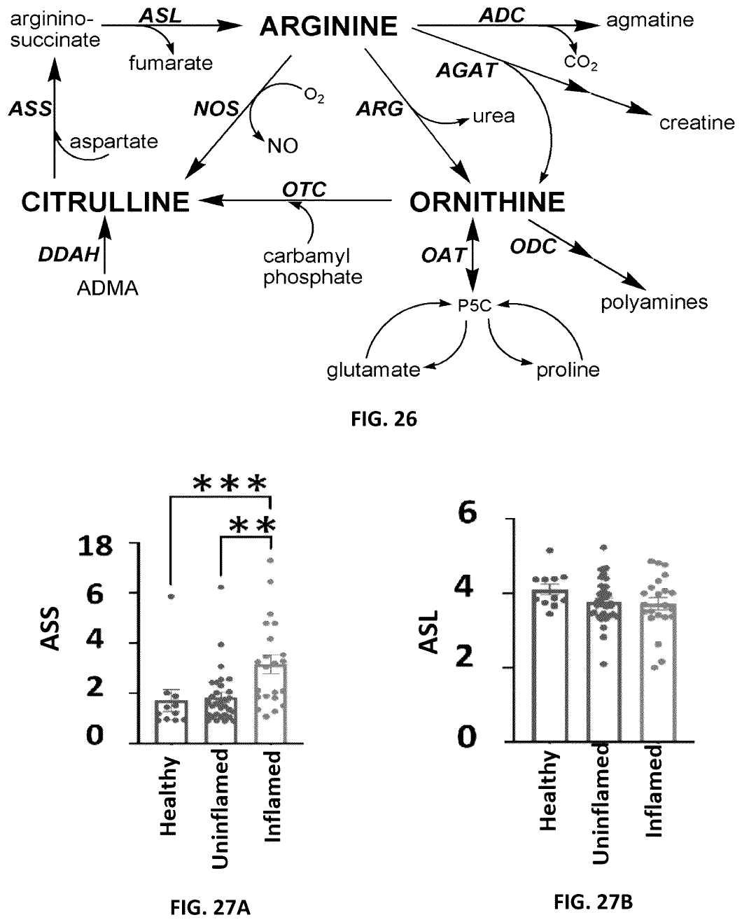

As established above, ASA when injected in mouse model of colitis (DSS) exacerbates weight loss (FIG. 18B). ASA is part of arginine/urea cycle as shown in FIG. 26. In view of this, and argininosuccinate synthetase's (ASS) potential as a biomarker for IBD, we analyzed the potential of ASA as a biomarker for IBD.

We analyzed in silico human IBD data (obtained from GSE179285) in patients with or without an inflamed colon and compared it to healthy controls. This data shows that ASS, which makes ASA, increases in the inflamed tissues when compared to both the healthy controls and IBD patients without an inflamed colon. (FIG. 27A). We also analyzed levels of ASL, which degrades ASA, which remained the same in all three groups. (FIG. 27B) This indicates that ASA concentrations will rise in IBD because the enzyme ASS that makes it increases with IBD, but the enzyme ASL that break it down does not.

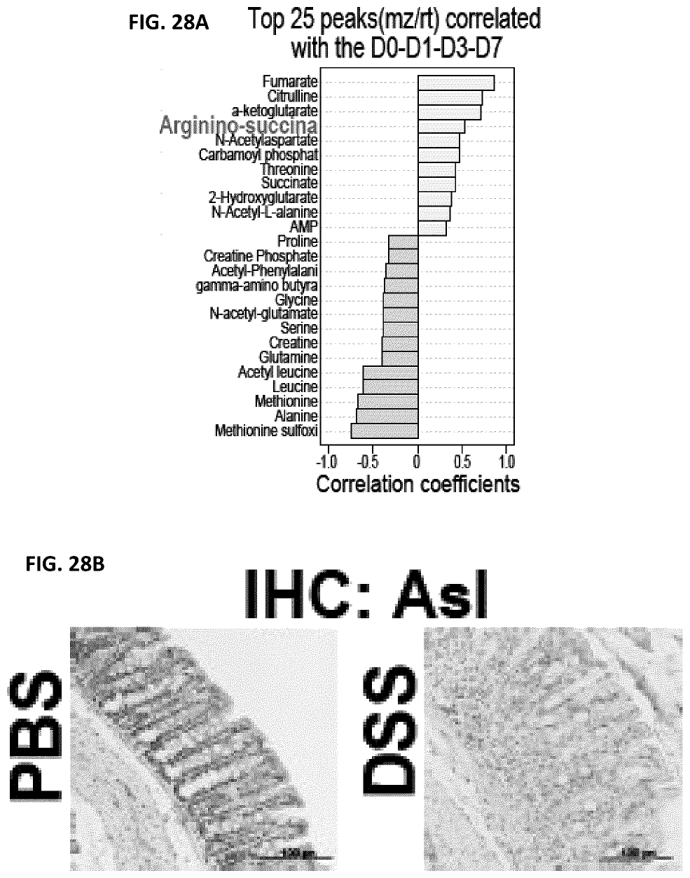

Next, we analyzed serum levels of various proteins, including ASA, in a mouse model of DSS colitis and compared it to a healthy mouse model treated with PBS alone. In the mouse model, serum levels of ASA were increased when compared to the healthy mouse. (FIG. 28A). Histology sections of each mouse were also stained for ALS. The DSS colitis model showed a marked reductions in ASL levels in the colon indicating that ASA levels rise because there is less enzyme to degrade ASA in colitis. (FIG. 28B)

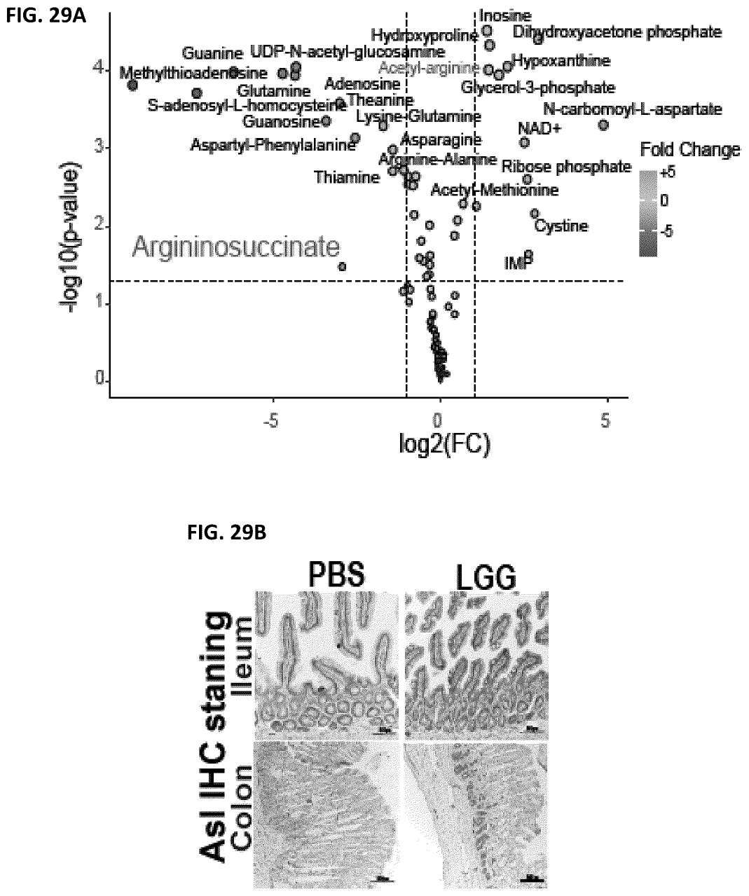

When LGG bacteria is cultured in bacteria growth media, ASA levels are significantly reduced. (FIG. 29A). Accordingly, we monoconolized germ-free mice with LGG so that no other bacteria were present in the gut and compared ASL levels in the small intestine (ileum) and the colon with ASL levels in germ-free mice administered only PBS. In the LGG mice, ASL levels were increased in both the small intestine and the colon. (FIG. 29B right panels) Accordingly, LGG enhances expression of the enzyme ASL to degrade ASA.

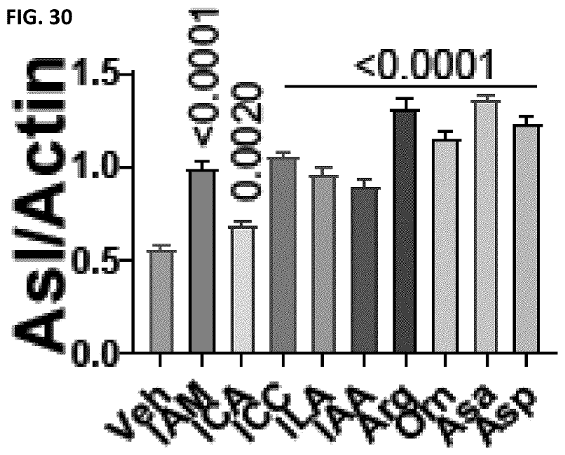

A similar experiment was repeated in human Caco2 cells. The cells culture was treated with various LGG-derived metabolites and compared to a control culture that was untreated. The ASL levels were measured by Western blot analysis. Virtually all of the LGG-derived metabolites increased ASL levels when compared to the control. (FIG. 30)

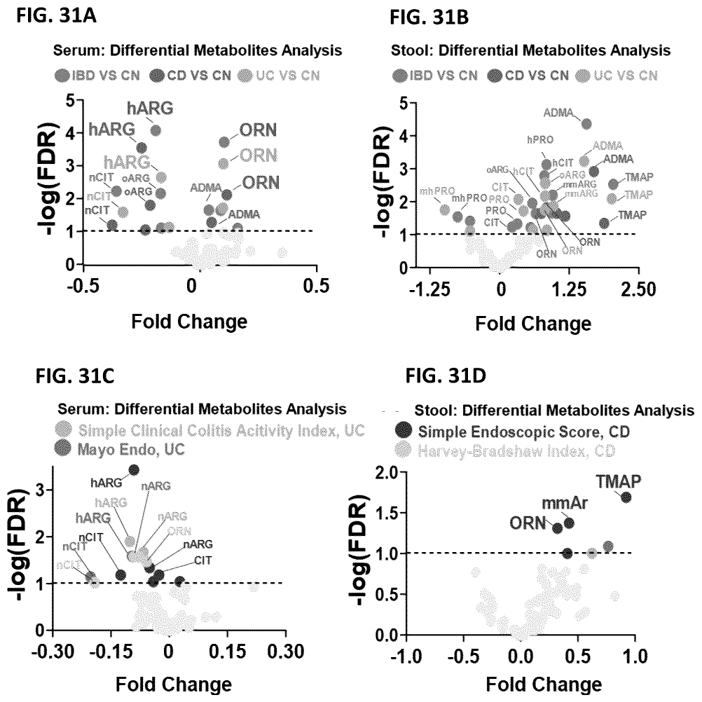

Next, we looked at existing Human IBD metabolite data which showed perturbed arginine metabolism. Serum homoarginine (hARG) is low in IBD while ornithine (ORN) is high. (FIG. 31A-31D). Accordingly, high serum levels for ORN is indicative of UC, CD or IBD. Additionally, high levels of asymmetric dimethyl arginine (ADMA) were observed in the stool and serum samples of IBD patients. These levels identify ADMA as another biomarker for IBD.

In summary, we have discovered novel metabolites that increase when the probiotic LGG is present in the gut. One family of metabolites (MNA, indoles) clearly prevent disruption of the gut barrier by harmful compounds like bacterial toxins. LGG increases levels of these metabolites.

Another family of metabolites (specifically ASA) is found in high levels in animal models of colitis and IBD patients and exacerbates leaky gut. This is supported by enzyme levels of related enzymes ASS and ASL. The enzyme ASS that makes ASA is found in high levels in IBD, while ASL that degrades ASA remains unchanged. LGG and its metabolites increase levels of ASL in the host, thereby reducing ASA in the serum.

While certain of the preferred embodiments of the present invention have been described and specifically exemplified above, it is not intended that the invention be limited to such embodiments. It will be apparent to one skilled in the art that various changes and modifications can be made therein without departing from the scope of the present invention, as set forth in the following claims.

### EMBODIMENTS

1. A method for diagnosing leaky gut in a subject comprising:
   (a) measuring in a biological fluid sample of the subject the expression level of a polynucleotide or oligonucleotide sequence, which hybridizes to a gene, gene fragment, gene transcript or expression product selected from the biomarkers identified in FIG. 8K, 9H, 14, and
   (b) comparing said subject's selected gene, gene fragment, gene transcript or expression product expression level with the level of the same gene, gene fragment, gene transcript or expression product in the biological fluid of a reference or control subject,
   wherein changes in expression of the subject's selected gene, gene fragment, gene transcript or expression products from those of the reference or control correlates with a diagnosis of leaky gut.
2. The method according to embodiment 1, wherein the biomarkers are selected from at least one of the group consisting of:
   i. L-argininosuccinic acid (ASA),
   ii. argininosuccinate synthase 1 (ASS1),
   iii. argininosuccinate lyase (ASL), and
   iv. Nitric oxide synthase (NOS2).
3. The method according to embodiment 1 or embodiment 2, wherein the biomarker is l-arginosuccinic acid (ASA).
4. The method according to embodiment 1 or embodiment 2, wherein the amino acid, peptides or proteins are all of biomarkers (i)-(iv).
5. The method according to any one of embodiments 1-4, wherein said change in expression level of each said selected gene, gene fragment, gene transcript or expression product comprises an upregulation in comparison to said reference or control or a downregulation in comparison to said reference or control.
6. A method for diagnosing leaky gut in a subject comprising:
   (a) measuring in a biological fluid sample of the subject the expression level of a protein, peptide fragment or expression product thereof selected from at least one of the biomarkers identified in FIG. 8K, 9H, 14,; and
   (b) comparing said subject's expression level of the selected biomarker with the level of the same biomarker in the biological fluid of a reference or control subject, wherein changes in expression of the subject's selected biomarker from those of the reference or control correlates with a diagnosis of leaky gut.
7. The method according to embodiment 6, wherein the biomarkers are selected from at least one of the group consisting of:
   i. L-argininosuccinic acid (ASA),
   ii. argininosuccinate synthase 1 (ASS1),
   iii. argininosuccinate lyase (ASL), and
   iv. Nitric oxide synthase (NOS2).
8. The method according to embodiment 6 or embodiment 7, wherein the biomarker is I-arginosuccinic acid (ASA).
9. The method according to any one of embodiments 1-8, wherein said change in expression level of each said selected biomarker comprises an upregulation in comparison to said reference or control or a downregulation in comparison to said reference or control.
10. The method according to any one of embodiments 1-9, further comprising: measuring
   i) the expression level of an expression product, protein or peptide fragment selected from FIG. 8K, 9H, 14, or
   ii) a polynucleotide or oligonucleotide sequence, which hybridizes to a gene, gene fragment, gene transcript or expression product selected from FIG. 8K, 9H, 14.
11. The method according to any one of embodiments 1-10, further comprising administering to the patient a therapeutic agent that reduces inflammation.
12. The method according to embodiment 11, wherein the agent is selected from at least one nutraceutical, anti-inflammatory drug, antibiotic, immunomodulator, anti-diarrheal medication, pain reliever, iron supplement, and calcium and vitamin D supplement.
13. The method according to either one of embodiments 11 or 12, wherein the agent is a nutraceutical.
14. The method according to embodiment 13, wherein the nutraceuticals are selected from one or more of the compounds listed in Tables 2 or 3.
15. The method according to any one of embodiments 13 or 14, wherein the nutraceutical is one or more of indole acetamide (IAM), methynicotinamide (MNA), carnosine (CARN), and indole-propionic acid (IPA).
16. The method according to embodiment 15, wherein the nutraceutical is all of IAM, MNA, CARN, and IPA.
17. The method according to embodiment 15, wherein the nutraceutical is MNA.
18. The method according to any one of embodiments 11-17, further comprising at least one additional therapeutic agent.
19. The method according to any one of embodiments 11-18, wherein the agent is administered orally, intrapertioneally, intravenously, or intramuscular.
20. The method of according to any one of embodiments 11-19, wherein the agent is administered
21. The method of according to any one of embodiments 11-19, wherein the agent is administered intraperitoneally.
22. The method according to anyone of embodiments 11-21, further comprising repeating steps a) and b) after administration of the therapeutic agent.
23. A method for treating an inflammatory disease in a subject in need thereof, the method comprising administering one or more of the nutraceutical compounds listed in Tables 2 or 3.
24. The method of embodiment 23, wherein the nutraceutical is one or more of indole acetamide (IAM), methynicotinamide (MNA), carnosine (CARN), and indole-propionic acid (IPA).
25. The method according to embodiment 24, wherein the nutraceutical is all of IAM, MNA, CARN, and IPA.
26. The method according to embodiment 24, wherein the nutraceutical is MNA.
27. The method according to any one of embodiments 23-26, further comprising administering at least one additional therapeutic agent.
28. The method according to any one of embodiments 23-27, wherein the agent is administered orally, intrapertioneally, intravenously, or intramuscular.
29. The method of according to any one of embodiments 23-28, wherein the agent is administered
30. The method of according to any one of embodiments 23-28, wherein the agent is administered intraperitoneally.
31. The method according to any one of the preceding embodiments, wherein the patient has Inflammatory Bowel disease and/or Type I Diabetes.
32. A method for treating a leaky gut syndrome in a subject in need thereof, the method comprising administering one or more of the nutraceutical compounds listed in Tables 2 or 3.
33. The method of embodiment 32, wherein the nutraceutical is one or more of indole acetamide (IAM), methynicotinamide (MNA), carnosine (CARN), and indole-propionic acid (IPA).
34. The method according to embodiment 33, wherein the nutraceutical is all of IAM, MNA, CARN, and IPA.
35. The method according to embodiment 33, wherein the nutraceutical is MNA.
36. The method according to any one of embodiments 32-35, further comprising administering at least one additional therapeutic agent.
37. The method according to any one of embodiments 32-36, wherein the agent is administered orally, intrapertioneally, intravenously, or intramuscular.
38. The method of according to any one of embodiments 32-37, wherein the agent is administered
39. The method of according to any one of embodiments 32-37, wherein the agent is administered intraperitoneally.
40. The method according to any one of the preceding embodiments, wherein the patient has Inflammatory Bowel disease and/or Type I Diabetes.
41. A kit for performing the method according to any one of the preceding embodiments.

1. A method for diagnosing leaky gut in a subject comprising:
(a) measuring in a biological fluid sample of the subject the expression level of
i) a polynucleotide or oligonucleotide sequence, which hybridizes to a gene, gene fragment, gene transcript or expression product selected from the biomarkers identified in FIG. 8K, 9H, 14, or
ii) a polypeptide comprising a protein, peptide fragment, or expression product selected from the biomarkers identified in FIG. 8K, 9H, 14, or and
(b) comparing said subject's expression level of the selected biomarker with the level of the same biomarker in the biological fluid of a reference or control subject,
wherein changes in expression of the subject's selected biomarker from those of the reference or control correlates with a diagnosis of leaky gut.

2. The method according to claim 1, wherein the biomarkers are selected from at least one of the group consisting of:
i. L-argininosuccinic acid (ASA),
ii. argininosuccinate synthase 1 (ASS1),
iii. argininosuccinate lyase (ASL), and
iv. Nitric oxide synthase (NOS2).

3. The method according to claim 1, wherein the biomarker is l-arginosuccinic acid (ASA).

4. The method according to claim 2, wherein the biomarkers are all of biomarkers (i)-(iv).

5. The method according to claim 1, wherein said change in expression level of each said selected biomarker comprises an upregulation in comparison to said reference or control or a downregulation in comparison to said reference or control.

6. The method according to claim 1, further comprising administering to the patient a therapeutic agent that reduces inflammation or a nutraceutical.

7. The method according to claim 6, wherein the agent is selected from at least one nutraceutical, anti-inflammatory drug, antibiotic, immunomodulator, anti-diarrheal medication, pain reliever, iron supplement, and calcium and vitamin D supplement.

8. The method according to claim 6, wherein the nutraceutical are selected from one or more of the compounds listed in Table 1A, 1B, 2, or 3.

9. The method according to claim 8, wherein the nutraceutical is one or more of indole acetamide (IAM), methynicotinamide (MNA), carnosine (CARN), and indole-propionic acid (IPA).

10. The method according to claim 9, wherein the nutraceutical is all of IAM, MNA, CARN, and IPA.

11. The method according claim 8, further comprising at least one additional therapeutic agent.

12. The method according to claim 8, wherein the agent is administered orally, intraperitoneally, intravenously, or intramuscular.

13. The method of according to claim 8, wherein the agent is administered orally.

14. The method according to claim 8, further comprising repeating steps a) and b) after administration of the therapeutic agent.

15. A method for treating a leaky gut syndrome in a subject in need thereof, the method comprising administering one or more of the nutraceutical compounds listed in Tables 1A, 1B, 2, or 3.

16. The method of claim 15, wherein the nutraceutical is one or more of indole acetamide (IAM), methynicotinamide (MNA), carnosine (CARN), and indole-propionic acid (IPA).

17. The method according to claim 16, wherein the nutraceutical is all of IAM, MNA, CARN, and IPA.

18. The method according to claim 15, further comprising administering at least one additional therapeutic agent.

19. The method according to claim 15, wherein the agent is administered orally, intraperitoneally, intravenously, or intramuscular.

20. The method according to claim 15, wherein the patient has Inflammatory Bowel disease, Chron's disease, ulcerative colitis, or Type I Diabetes.
